(19) United States
(12) Patent Application Publication
(10) Pub. No.: US 2025/0179211 A1
(43) Pub. Date: Jun. 5, 2025

(54) Antibodies and antibody fragments and analogues specific for chondroitin sulfate

(71) Applicant: VAR2 Pharmaceuticals ApS, Copenhagen N (DK)

(72) Inventors: Ali EL-Salanti, Farum (DK); Swati Choudhary, Copenhagen S (DK); Jan Tobias Gustafsson, Hjärup (SE); Robert Dagil, Skovlunde (DK); Elena Ethel Vidal-Calvo, Copenhagen V (DK); Mads Daugaard, Vancouver, British Columbia (CA); Mette Ørskov Agerbæk, Valby (DK)

(21) Appl. No.: 18/836,281

(22) PCT Filed: Feb. 7, 2023
(86) PCT No.: PCT/EP2023/052941

§ 371 Date: Aug. 6, 2024

(30) Foreign Application Priority Data
Feb. 7, 2022 (EP) 22155453.8

(51) Int. Cl. C07K 16/30; A61K 39/00; A61K 40/11; A61K 40/31; A61K 40/42; A61K 47/68; A61P 35/00; C07K 14/55; C07K 16/28; C12N 5/0783; G01N 33/574

(52) U.S. Cl. CPC C07K 16/3053; A61K 40/11; A61K 40/31; A61K 40/4261; A61K 47/68031; A61K 47/6865; A61P 35/00; C07K 14/55; C07K 16/2809; C12N 5/0636; G01N 33/57484; A61K 2039/505; C07K 2317/31; C07K 2317/35; C07K 2317/622; C07K 2317/73; C07K 2317/92; C07K 2319/00; C12N 2510/00; G01N 2333/4722

(57) ABSTRACT

Provided is antibodies and antibody variants that specially bind oncofetal chondroitin sulfate. Also provided is conjugates, fusion proteins, and CAR-T cells comprising the antibodies or antibody variants, as well as methods and use of these agents for therapeutic and diagnostic purposes, in particular for treatment and diagnosis of cancer.

### FIELD OF THE INVENTION

The present invention relates to antibodies and antibody variants specifically targeting oncofetal chondroitin sulfate (ofCS) as well as to means and methods for their preparation and use.

### BACKGROUND OF THE INVENTION

CS belongs to the family of glycosaminoglycans (GAGs), which are long unbranched disaccharide repeat units attached to protein cores unified as chondroitin sulfate proteoglycans (CSPGs). CSPGs are located intracellularly, on the cell membrane, or in the extracellular matrix (ECM). CSPGs can contain one or several GAG-chains. GAGs are divided into six distinct classes based on the identity of the repeating disaccharide units: chondroitin sulfate (CS, hereunder CSA, CSB, and CSC), heparin, heparan sulfate (HS), keratin sulfate, and hyaluronan. CSA/C and CSB both have the monosaccharide unit N-acetylgalactosamine (GalNAc) and a uronic acid residue, but this residue differs in chirality, giving either glucuronic acid (GlcA) for CSA/C or iduronic acid (IdoA) for CSB. A GAG chain predominantly consisting of CSB is also sometimes termed dermatan sulfate. The CS disaccharide units can be modified by the addition of sulfate groups, such as sulfation of carbon-2 of GlcA, and/or carbon-4 (C4S) and carbon-6 (C6S) of GalNAc. CSA is characterized by having C4S and CSC by having C6S GalNAc modifications. Further heterogeneity and specificity can arise from hybrid structures consisting of chains of for example CSA with intra-dispersed islands of CSB. We here refer to CS as covering CSA, CSB, and CSC and hybrid structures thereof. In particular, the CSPGs of C4S and C6S-type (also called CSA/CSC) have been described in various malignancies whereas CSA/CSC-CSB hybrid chains have been described to provide optimal sequence for binding of the growth factor pleiotrophin during embryonic development and has also been described in various malignancies. Abnormal expression of CSPGs has been directly linked to most forms of cancer. Chondroitin Sulfate Proteoglycan 4 (CSPG4), CD44 (CSPG8), and versican (CSPG2), are pro-malignancy membrane CSPGs. Specific CSPG expression patterns have also been observed in different cancers. For example, CSPG4 is highly expressed in many tumour cells. CD44 is one of the most used biomarkers for breast cancer stem cells and high levels of versican have been associated with various malignancies. CSPGs bind chemokines and promote proliferation, migration and invasion of both primary tumours and the metastases by consolidating growth factor receptor complexes and extracellular matrix components to transmit pro-oncogenic signalling events. Accordingly, CSPGs have been and are widely exploited as targets for cancer therapy as well as for diagnostic purposes. The main focus of therapeutic strategies has been on targeting the protein core of CSPGs, and little attention has been given to the carbohydrate part of CSPGs. This is mostly because the characterization of oligosaccharides is technically challenging and antibodies that can distinguish different CS structures are lacking. A few published and used CS antibodies only bind the first GalNAc-GlcA stub attached to the protein core (e.g., 2B6 or 3B3), which appears after enzymatic chondroitinase treatment, and are thus not clinically relevant from a therapeutic targeting point of view (Davies et al., Osteoarthritis and Cartilage, 2008; Hardingham et al., Carbohydrate Research, 1994). Other CS antibodies, mainly low affinity IgMs (e.g., CS-56 or PG-4), recognize simple structures and have accordingly also not been demonstrated to be able to specifically target tumours in vivo (Detamore et al., Matrix Biology, 2005; Garciá-Piqueras et al., Anat. Rec. (Hoboken), 2019). Therefore, in lack of highly specific and high affinity antibody reagents to the tumour-associated sugar modification, people have focused on the different protein backbones themselves, as proteoglycans in many cases are also overexpressed in cancer.

The narrow focus on the protein part of CSPGs is problematic if the malignant functions rely on the nature of the CS displayed by the CSPG. A malignant CS may be redundantly displayed on several different protein cores and targeting a single protein could have limited potential. Furthermore, this could allow for treatment resistance to occur, as the pleiotropic and functional determinant CS could be shifted to protein cores not targeted by the therapy. An increasing number of studies indicate that the functions of CSPGs are exerted through an interplay between the specific CS-chain motif and the core protein scaffold. Two examples are versican and CSPG4, which act as ligands for P- and L-selectins expressed on the endothelium. These interactions are mediated by CS and can contribute to the tumour metastatic processes. Another example is the role of CS in the activation of an ECM degrading enzyme, metalloproteinase-2 (MMP-2). Activation of MMP-2 is associated with enhanced invasive and metastatic properties of cancers, and 4-O-sulfated CS on CSPG4 in melanoma was found to increase the activation of MMP-2. Only few CS-motifs have been identified so far, primarily due to limitations in the structural analysis of specific GAG-chains, which typically cannot provide information on the exact sequence, but only on the overall disaccharide composition.

VAR2CSA—an ofCS binding malaria protein: During pregnancy, infection with the parasite Plasmodium falciparum can cause placental malaria. The parasite infects erythrocytes and these infected cells are filtered and cleared in the spleen. To evade clearance, the parasite expresses proteins on the surface of the infected erythrocytes that effectively anchor these cells to the vascular wall. Parasites accumulate in placenta because parasites can bind to CSPGs expressed on the placental syncytiotrophoblast. VAR2CSA, the parasite protein mediating the binding, has evolved to facilitate high-affinity binding to placental CS-containing CSPGs. We discovered VAR2CSA in 2003 and have subsequently mapped and characterised the CS binding domain of VAR2CSA. Recombinant sub-fragments of VAR2CSA (rVAR2) bind ofCS with affinity in the low nanomolar range (1-10 nM). The binding is highly specific, as rVAR2 does not bind to carbohydrates with a sulfation pattern distinct from that found on placental CS. This specific interaction to distinct placental CS is the result of natural selection of VAR2CSA-expressing parasites binding to placenta tissue, as this is the only place in the vasculature that we find this particular CS. Hence parasites expressing VAR2CSA only sequester in the placenta. The placenta is a fast-growing organ in which the cells display a high mitosis rate, the ability to invade into the uterine tissue, and to evade the immune system. CS-containing CSPGs are abundantly expressed by trophoblasts and attract chemokines and nutrients to ensure high division rates of the placental cells. The placental cell expression of CSPGs with a unique form of CS chains likely reflects the specific need of these cells to migrate and divide rapidly—features shared with cancer cells. We therefore speculated that cancer cells turn on the expression of a distinct oncofetal CS (ofCS) to facilitate rapid growth and metastatic spread.

We have tested the binding of rVAR2 to hundreds of cancer cell lines and found that rVAR2 reacted with more than 95% of patient-derived human cancer cell lines, while non-cancer cells did not bind rVAR2. We examined a panel of human tumours for rVAR2 binding as compared to matching adjacent normal tissue from the same patients. All tumours displayed strong rVAR2 staining and only weak staining in matched normal tissue. Binding of rVAR2 to primary human tumours could be completely inhibited by enzymatic removal of CS from the tissue. In larger cohorts, rVAR2 tissue binding strongly correlated with progression in patients with malignant melanoma, poor relapse-free survival in non-small cell lung cancer patients, and poor outcome in patients with bladder cancer. Mass spectrometry of rVAR2 pull-downs from cancer cells and tissues demonstrate that a wide range of proteins are modified with an ofCS glycosylation, and in particular proteins involved in growth and cellular migration.

After intravenous (IV) injection of fluorescent labelled rVAR2 to a wide range of tumour bearing mice (xenograft, allograft, PDX), rVAR2 rapidly locates to the tumour tissue. Similarly, we showed that that IV administration of rVAR2 drug conjugate was not associated with adverse effects and was able to reduce tumours in different experimental models, including prostate cancer, orthotopic bladder cancer, lymphoma, metastatic breast cancer, melanoma and orthotopic PDX models of glioblastoma. Testing blood samples from cancer patients, we showed that rVAR2 immobilized on magnetic beads captured circulating tumour cells (CTCs) from blood samples from all tested cancer patients (pancreatic, prostate, lung, glioma, melanoma, breast, and liver cancers) even at a very early stage. We have developed a technology to capture circulating tumour cells (CTCs) in blood samples using rVAR2 coated magnetic beads. These data demonstrated the universal cancer cell expression of ofCS and clinical feasibility in targeting ofCS (Salanti and Clausen et al., Cancer Cell, 2015; Agerbæk et al., Nat. Commun., 2018).

Importantly, these clinical data demonstrate the broad cancer cell expression of ofCS. Published data show key information on the ofCS structure. In summary, these data demonstrate that ofCS consists of a complex hybrid structure of both 4 (CSA) and 6 (CSC) sulfated CS and IdoA molecules (i.e., dermatan sulfate DS or CSB). Further data has demonstrated that the ofCS chains associated with placental development and cancer are unusually overexpressed and particularly long. This was consistent with footprint analyses of the rVAR2-ofCS binding interface showing that rVAR2 binds a long stretch of oligosaccharides, which likely determines the oncofetal trophism (Spliid et al., J. Biol. Chem., 2021).

In summary, ofCS is highly expressed both on the surface of the cancer cells and in the ECM of the tumour tissue and absent in normal tissues, thus providing an attractive cancer target. Clearly, rVAR2 can be used to target ofCS on cancers and cancer cells, and the protein could potentially form the basis for new drugs and diagnostics. However, using recombinant malarial VAR2 protein for therapeutic purposes has proven challenging. Upon IV injection the recombinant protein has a plasma half-life of less than 5 minutes due to liver clearance. Thus, treatment of tumours may require substantial amounts of protein and repeat injections. Another consideration is that rVAR2 is a foreign malaria protein, and at some point, an antibody response will be induced that might neutralize the treatment. Finally, the recombinant malaria protein is a large and complex protein with hundreds of cysteines and many interdomain disulfide bonds, making production and stability a challenge. An antibody with the same specificity as rVAR2 would circumvent risks associated with rVAR2 production, immunogenicity, and serum half-life. An anti-ofCS monoclonal antibody (mAb) might even be effective in itself without toxin conjugation as it would be present in the plasma for extended periods of time and potentially neutralize CTCs in transit to a pre-metastatic niche.

### OBJECT OF THE INVENTION

It is an object of embodiments of the invention to provide antibodies and antibody variants that bind specifically to ofCS, and to provide means, methods and uses for therapy and diagnosis of cancer.

### SUMMARY OF THE INVENTION

Prior art antibodies directed to CS are of the IgM type and generally of low affinity and cannot distinguish between ofCS overexpressed on cancer cells and other glycosaminoglycans like heparan sulfate, hyaluronic acid or simple CSA structures, whereas it has been found by the present inventors that the antibodies and antibody variants provided herein all have the ability to discriminate between ofCS and other GAGs by having a high specificity towards ofCS thus making their binding properties comparable to those of VAR2CSA.

So, in a 1^{st} aspect, the present invention relates to an antibody or an antibody variant that binds specifically to a chondroitin sulfate (CS) glycosaminoglycan chain, wherein the antibody or the antibody variant exhibits a higher binding affinity for oncofetal CS (ofCS) than for non-oncofetal glycosaminoglycans, such as for heparan sulfate and for hyaluronic acid.

In a 2^{nd} aspect, the present invention relates to a conjugate or a fusion protein comprising at least a first and a second moiety, wherein the first moiety is the antibody or the antibody variant according the first aspect of the invention or any embodiment thereof disclosed herein, and the second moiety is a molecule or polypeptide; wherein the first moiety is conjugated or genetically fused to the second moiety, and wherein the second moiety provides or improves the therapeutic and/or diagnostic function of the conjugate or the fusion protein.

In a 3^{rd} aspect, the present invention relates to a polypeptide comprising i) a first polypeptide domain being a transmembrane domain and an endodomain of a chimeric antigen receptor (CAR), and ii) a second polypeptide domain being the antibody or the antibody variant according to the 1^{st} aspect of the invention or any embodiment thereof disclosed herein.

In a 4^{th} aspect, the present invention relates to a CAR-T cell comprising the polypeptide of 3^{rd} aspect of the invention or any embodiment thereof disclosed herein.

In a 5^{th} aspect, the present invention relates to an isolated nucleic acid molecule comprising a nucleotide sequence encoding the antibody or the antibody variant of the 1^{st} aspect of the invention or any embodiment thereof disclosed herein, the conjugate or the fusion protein of the 2^{nd} aspect of the invention or any embodiment thereof disclosed herein, or the polypeptide of the 3^{rd} aspect of the invention or any embodiment thereof disclosed herein.

In a 6^{th} aspect, the present invention relates to a vector comprising the isolated nucleic acid molecule of the 5^{th} aspect of the invention or any embodiment thereof disclosed herein, such as an expression vector or a cloning vector.

In a 7^{th} aspect, the present invention relates to a host cell comprising or transformed with the vector of the 6^{th} aspect of the invention or any embodiment thereof disclosed herein.

In an 8^{th} aspect, the present invention relates to a method for producing the antibody or the antibody variant according to the 1^{st} aspect of the invention or any embodiment thereof disclosed herein, the conjugate or the fusion protein of the 2^{nd} aspect of the invention or any embodiment thereof disclosed herein, or the polypeptide of the 3^{rd} aspect of the invention or any embodiment thereof disclosed herein, the method comprising the steps of: transfecting or transforming a host cell with the vector of the 6th aspect of the invention or any embodiment thereof disclosed herein, expressing the nucleotide sequence according to the 5th aspect of the invention or any embodiment thereof disclosed herein, and isolating the antibody or the antibody variant, the conjugate or the fusion protein, or the polypeptide.

In a 9^{th} aspect, the present invention relates to a pharmaceutical composition comprising the antibody or the antibody variant of the 1^{st} aspect of the invention or any embodiment thereof disclosed herein, the conjugate or the fusion protein of the 2^{nd} aspect of the invention or any embodiment thereof disclosed herein, or the CAR-T cell of the 4^{th} aspect of the invention or any embodiment thereof disclosed herein, and a pharmaceutically acceptable carrier, vehicle or diluent.

In a 10^{th} aspect, the present invention relates to use of the antibody or the antibody variant of the 1^{st} aspect of the invention or any embodiment thereof disclosed herein, or the conjugate or the fusion protein of the 2^{nd} aspect of the invention or any embodiment thereof disclosed herein, for in vitro detection and/or isolation of cancer cells, such as cancer cells derived from a subject, such as circulating tumor cells.

In an 11^{th} aspect, the present invention relates to the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein, for use as a medicament.

In a 12^{th} aspect, the present invention relates the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein, for use in diagnosing cancer in a subject.

In a 13^{th} aspect, the present invention relates to the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein, for use in treating cancer in a subject in need thereof, or for treating a condition involving expression, such as inappropriate expression, of ofCS, such as in a condition selected from the group consisting of arthritis, arthrosis, multiple sclerosis, healing after neural damage, cartilage repair, wound healing, and psoriasis, or for delivering a therapeutic to the placenta in subject in need thereof.

In a 14^{th} aspect, the present invention relates to a method of treating a cancer, or treating a condition involving expression, such as inappropriate expression, of ofCS, such as in a condition selected from the group consisting of arthritis, arthrosis, multiple sclerosis, healing after neural damage, cartilage repair, wound healing, and psoriasis, or for delivering a therapeutic to the placenta, in a subject in need thereof, the method comprising administering to the subject a therapeutically effective amount of the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein.

In a 15^{th} aspect, the present invention relates to a method for detection of ofCS in a sample, the method comprising
1) contacting said sample with an antibody or antibody variant of the 1^{st} aspect of the invention or any embodiment thereof disclosed herein, to allow the antibody or antibody variant to specifically bind ofCS, subsequently contacting the antibody or antibody variant and any ofCS bound thereby with an agent, which is capable of binding ofCS, to allow said agent to bind ofCS, or
2) contacting said sample with an agent, which is capable of binding ofCS, to allow the agent to bind ofCS, subsequently contacting the agent and any ofCS bound thereby with an antibody or antibody variant of the 1^{st} aspect of the invention or any embodiment thereof disclosed herein, to allow the antibody to bind ofCS,
and detecting quantitatively or qualitatively the presence of complexes comprising the ofCS, the antibody or antibody variant, and the agent,
   wherein the agent and the antibody or antibody variant are non-competitive for their binding to ofCS.

In a 16^{th} aspect, the present invention relates to a detection kit comprising an antibody of the 1^{st} aspect of the invention or any embodiment thereof disclosed herein, an agent as defined in the 14^{th} aspect of the invention or any embodiment thereof disclosed herein, and a reaction vessel, and optionally also a labelled agent, which binds the antibody or agent.

In a 17^{th} aspect, the present invention relates to a method for providing nucleic acids encoding the antibody or the antibody variant of the 1^{st} aspect of the invention or any embodiment thereof disclosed herein, comprising screening a library comprising antigen-binding fragments of antibodies, wherein the library is comprised of display agents, which are selected from cells, virus, and phage comprising antibody-encoding nucleic acid fragments, for binding of the display agents to a capture agent consisting essentially of purified ofCS or ofCS-decorated protein, and subsequently isolating antibody coding nucleic fragments from the display agents, which bind the capture agent, and optionally sequencing the isolated nucleic acid fragments.

Finally, in a 18^{th} aspect, the present invention relates to a method of producing an antibody or antibody variant of the 1^{st} aspect of the invention or any embodiment thereof disclosed herein, comprising expressing an expression vector comprising a nucleic acid fragment obtained according to the method of the 16^{th} aspect of the invention or any embodiment thereof disclosed herein, in a host cell in a culture and subsequently isolating the expression product from the culture.

### LEGENDS TO THE FIGURES

FIG. 1: Binding of selected ofCS IgG antibodies to ofCS, with and without chondroitinase treatment (B3 binding after chondroitinase-treatment was undetectable), as measured by ELISA.

FIG. 2: Binding of selected ofCS scFv antibodies to ofCS, with and without chondroitinase treatment, as measured by ELISA.

FIG. 3: Binding of the ofCS antibody B3 and commercially available anti-CS antibodies to ofCS, ofCSPG on a decorin backbone, and commercially available purified cartilage decorin, as determined by ELISA.

FIG. 4: Binding of selected ofCS IgG antibodies to Lymphoma cancer cells (Karpas), in the presence and absence of soluble CSA (binding of F2, F11, B3, and VAR2CSA is undetectable in the presence of soluble CSA), as determined by flow cytometry normalized to background binding of secondary antibody.

FIG. 5: Staining of bladder cancer, placenta, and non-cancer liver tissue sections with the ofCS scFv antibody variant C9, with and without chondroitinase treatment (C-ase), as determined by immunohistochemistry (IHC).

FIG. 6: In vivo imaging system (IVIS) CT images of two mice with engrafted tumours in their right flank. The image shows the distribution of a NIR-labeled ofCS IgG C9 at different time intervals after its tail vein injection. Clearly the anti-ofCS antibody locates to the tumour allografted on the right flank of the mouse. The mouse on the right side was not injected with antibody but was included to determine autofluorescence.

FIG. 7: 200,000 luciferase-positive 4T1 tumour cells were injected into immunocompetent mice after preincubation with 50 μg C9 IgG, ctrl antibody or PBS. Tumour growth in the lungs was monitored using IVIS scanning after luciferin injection, and tumour occurrence was plotted as survival curve adjusted to a percentage risk of getting a tumour dependent on treatment regimen.

FIG. 8: 4T1 tumour cells were injected into mice and treated peri-tumourally (PT) with a VAR2-anti-CD3 or an ofCS scFv (C9)-anti-CD3 bispecific molecule or PBS. The figure shows tumour growth over time.

FIG. 9: Different amounts of the CSPG rEndocan was spiked into buffer, and the ability of selected ofCS scFvs to capture the CSPG was measured by ELISA by detection of captured endocan using an anti-endocan antibody.

FIG. 10: Line graphs showing tumour volume of CT26 colon carcinoma in mice treated with control (left) and antibody C9 (right).

FIG. 11: Line graphs showing tumour volume of Karpas lymphoma tumours in mice treated with control (left) and antibody C9 (right).

FIG. 12: Tissues from tumours and normal corresponding tissue stained with DAPI (panels A and B) and antibody C9 (panels C and D).

FIG. 13: Graphs showing fluorescence from tumour cells treated with either spyCAR+ T cells and C9 scFv or with non-transduced T-cells and C9 scFv.

FIG. 14: Schematic depictions of various construct formats each comprising the C9 CDRs (SEQ ID Nos: 122-127) and corresponding graphs showing K_{D} values and binding kinetics curves.

### DETAILED DISCLOSURE OF THE INVENTION

### Definitions

An “antibody”, which is also termed an “immunoglobulin”, is a protein, which in humans consists of pairwise identical light chains and heavy chains, where the heavy and light chains each comprise a variable domain and a light domain, wherein the variable domains are responsible for the antibody's specific binding to an antigen. Generally, antibodies are grouped into the immunoglobulin groups IgG, IgM, IgA, IgE, and IgD, which each play different roles as antigen-recognizing agents in the immune system. The specificity for antigen binding of an antibody is determined by variable regions in the variable domains, and in particular by the complementarity determining regions (CDRs) in the variable domains. Generally, antibodies are molecules, which in principle or in practice can be a naturel expression product in an animal, meaning that an antibody includes all structural elements found in a naturally occurring immunoglobulin.

An “antibody variant” is a protein derived from an antibody, which has the same binding specificity as an antibody, but which would not be a natural expression product in a mammal. As such, the term refers to various fragments of antibodies as well as artificial antibody analogue formats. Also, the term denotes antibody formats that are found in nature but which are uncommon among mammals, such as single chain antibodies known from llamas and camels, and IgY found in birds and reptiles, but where CDRs from mammalian antibodies or combinatorically produced antibodies have been engineered into an antibody format from which it is not originally derived.

Oncofetal chondroitin sulfate (ofCS) is a generally highly sulfated form of chondroitin sulfate, which is found in placental tissue and on a large number of cancer cells. Its characteristics include a high degree of sulfation on the GalNAc residues of the CS chain, in particular by a majority of disaccharides having 4-O or 6-O sulfate groups on the GalNAc, but often (but not necessarily) also by the presence of at least one non-sulfated GalNac and the presence of 6-O sulphated GalNac as well as a L-iduronic acid unit. In some cases, the ofCS has a fully 6-O suflfated GalNac and no L-iduronic acid. The second unit of the ofCS disaccharide can be either a GlcA or a IdoA (L-iduronic acid) and along the ofCS chain these structures can be present as alternating hybrid structures or islands of CSA/C and CSB. Further complexity of the ofCS structure can be comprised of 2-O sulfation of the GalNac or IdoA saccharide.

### SPECIFIC EMBODIMENTS OF THE INVENTION

### 1^{st} Aspect of the Invention and Embodiments Thereof

This aspect relates to an antibody or an antibody variant that binds specifically to a chondroitin sulfate (CS) glycosaminoglycan chain, wherein the antibody or the antibody variant exhibits a higher binding affinity for oncofetal CS (ofCS) than for non-oncofetal glycosaminoglycans, such as for heparan sulfate and for hyaluronic acid.

### Preferred Antibodies of the 1^{st} Aspect are Those that

compete for binding to the CS glycosaminoglycan chain with VAR2CSA (SEQ ID NO: 135) or a CS binding fragment thereof, such as the polypeptide having SEQ ID NO: 136, and/or
competes for binding to the CS glycosaminoglycan with an scFv having any one of SeQ ID NOs: 1, 8, 15, 22, 29, 36, 43, 50, 51, 58, 65, 72, 79, 86, 93, 100, 107, 114, 121, and 128.

In other words, the antibody of the invention is preferably one that has the same binding specificity towards ofCS as the antibody specificities exemplified herein and/or as VAR2CSA.

As demonstrated in the examples, the antibodies and antibody variants exemplified do not all compete for binding to the same epitope on ofCS, which provides for the advantage of targeting different epitopes on ofCS, which provides advantages in a number of immune assay formats, but which also opens the door for therapy where several targets on ofCS can be exploited simultaneously.

As also shown in the examples, the antigen binding site of the identified antibodies is characterized by a distinctive positively charged groove—hence, the binding site of the antibody or variant is in important embodiments characterized by the presence of a positivity charged groove along the VH/VL boundary.

The positively charged groove can be constituted by positively charged and surface exposed amino acid residues (i.e., Lys, Arg, or His) present in the variable loops of both the V_{L} and V_{H}domain, typically 3, 4, 5, or 6 positively charged amino acid residues. The distance between the a carbon atoms of the positively charged amino acids across the V_{H}/V_{L} boundary is preferably at most 12 Å, such as in the range 6-12 Å, preferably between 6.7 and 11.4 Å, cf. the examples for details.

As shown herein, the exemplified antibodies all exhibit a higher affinity for ofCS than the commercially available CS antibodies 2H6, CS56, BE-123, and PG-5; this is a hallmark of the antibodies and variants of the present invention, so this affinity difference can be effectively used to distinguish the ofCS specific antibodies from known CS antibodies which do not find use as broad cancer diagnostics or broad therapeutics. Further, it is believed that part of the low affinity for ofCS exhibited by these commercially available antibodies is ascribable to the fact that they are all IgM antibodies—hence it is preferred that antibodies of the present invention are different from IgM.

Another characteristic of the antibodies of the 1^{st} aspect of the invention is their ability to bind directly to native ofCS, whereas at least some of 2H6, CS56, BE-123, and PG-5 require that the ofCS is treated with chondroitinase in advance. Hence preferred antibodies and variants of the 1^{st} aspect bind to native ofCS and will therefore not require treatment with chondroitinase in order to bind with high affinity.

While the antibody or the antibody variant of the 1^{st} aspect in principle can have any relevant format (that is, an IgG, IgE, IgD, and IgA, and with the above-indicated reservations, also IgM format), it is particularly preferred that the antibody or variant is or is derived from, an IgG antibody; the IgG can be of any subclass, i.e., IgG1, IgG2, IgG3, and IgG4. At any rate, the preferred antibodies or variants are or are derived from a human antibody, thus the antibody is preferably a human antibody, a humanized antibody, or an antibody variant, which has a pharmaceutically acceptable low immunogenicity when administered to humans. In line with the above considerations, the preferred antibody or variant of the 1^{st} aspect is one, which competes for binding to ofCS with a second antibody, wherein the second antibody comprises a heavy chain variable region (V_{H}) with an amino acid sequence present in any one of SEQ ID NOs: 1, 8, 15, 22, 29, 36, 43, 50, 51, 58, 65, 72, 79, 86, 93, 100, 107, 114, 121, and 128, and comprises a light chain variable region (V_{L}) with an amino acid sequence present in any one of SEQ ID NOs: 1, 8, 15, 22, 29, 36, 43, 50, 51, 58, 65, 72, 79, 86, 93, 100, 107, 114, 121, and 128.

In particularly preferred embodiments, the antibody or antibody variant of the 1^{st} aspect is one, wherein
the V_{H} region has
   an amino acid sequence present in or identical to the amino acid stretch which is linked C-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, or
   an amino acid sequence present in or identical to the amino acid stretch, which is linked C-terminally to the sequence GGGGSGGGGSGGGGSGGG (SEQ ID NO: 138) in SEQ ID NO: SEQ ID NO: 121,
and/or
   wherein the V_{L} region has
   an amino acid sequence present in or identical to the amino acid stretch which is linked N-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, or
   an amino acid sequence present in or identical to the amino acid stretch, which is linked N-terminally to the sequence GGGGSGGGGSGGGGSGGG (SEQ ID NO: 138) in SEQ ID NO: 121.

In some preferred embodiments closely linked to the examples, the antibody or the antibody variant comprises
a light chain complementarity determining region (LCDR)1 with the sequence of SEQ ID NO:2, 9, 16, 23, 30, 37, 44, 52, 59, 66, 73, 80, 87, 94, 101, 108, 115, 122, or 129, or a sequence having at least 70% sequence identity with any of these sequences, a LCDR2 with the sequence of SEQ ID NO: 3, 10, 17, 24, 31, 38, 45, 53, 60, 67, 74, 81, 88, 95, 102, 109, 116, 123, or 130, or a sequence having at least 70% sequence identity with any of these sequences, a LCDR3 with the sequence of SEQ ID NO: 4, 11, 18, 25, 32, 39, 46, 54, 61, 68, 75, 82, 89, 96, 103, 110, 117, 124, or 131, or a sequence having at least 70% sequence identity with any of these sequences, a heavy chain complementarity determining region (HCDR)1 with the sequence of SEQ ID NO: 5, 12, 19, 26, 33, 40, 47, 55, 62, 69, 76, 83, 90, 97, 104, 111, 118, 125, or 132, or a sequence having at least 70% sequence identity with any of these sequences, a HCDR2 with the sequence of SEQ ID NO: 6, 13, 20, 27, 34, 41, 48, 56, 63, 70, 77, 84, 91, 98, 105, 112, 119, 126, or 133, or a sequence having at least 70% sequence identity with any of these sequences, and a HCDR3 with the sequence of SEQ ID NO: 7, 14, 21, 28, 35, 42, 49, 57, 64, 71, 78, 85, 92, 99, 106, 113, 120, 127, or 134, or a sequence having at least 70% sequence identity with any of these sequences.

And, in certain embodiments, the antibody or the antibody variant preferably comprises a paratope defined by the following combination of amino acid sequences of LCDR1, LCDR2, LCDR3, HCDR1, HCDR2, and HCDR3, respectively:
SEQ ID NOs: 2, 3, 4, 5, 6, and 7; or
SEQ ID NOs: 9, 10, 11, 12, 13, and 14; or
SEQ ID NOs: 16, 17, 18, 19, 20, and 21; or
SEQ ID NOs: 23, 24, 25, 26, 27, and 28; or
SEQ ID NOs: 30, 31, 32, 33, 34, and 35; or
SEQ ID NOs: 37, 38, 39, 40, 41, and 42; or
SEQ ID NOs: 44, 45, 46, 47, 48, and 49; or
SEQ ID NOs: 52, 53, 54, 55, 56, and 57; or
SEQ ID NOs: 59, 60, 61, 62, 63, and 64; or
SEQ ID NOs: 66, 67, 68, 69, 70, and 71; or
SEQ ID NOs: 73, 74, 75, 76, 77, and 78; or
SEQ ID NOs: 80, 81, 82, 83, 84, and 85; or
SEQ ID NOs: 87, 88, 89, 90, 91, and 92; or
SEQ ID NOs: 94, 95, 96, 97, 98, and 99; or
SEQ ID NOs: 101, 102, 103, 104, 105, and 106; or
SEQ ID NOs: 108, 109, 110, 111, 112, and 113; or
SEQ ID NOs: 115, 116, 117, 118, 119, and 120; or
SEQ ID NOs: 122, 123, 124, 125, 126, and 127; or
SEQ ID NOs: 129, 130, 131, 132, 133, and 134,
where each of the sequences independently can also be a sequence having at least 70% sequence identity with the SEQ ID NO: indicated.

The above-indicated sequence identities may be higher, e.g. selected from at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, and at least 99%.

Sequence identity is in this context determined by optimal pairwise global alignment using the Needleman-Wunsch algorithm with the following parameters:
Substitution Matrix: EBLOSUM62
Gap penalty: 10.0
Extend penalty: 0.5

Some preferred antibodies or antibody variants of the 1^{st} aspect comprise a combination of
a V_{H} region having an amino acid sequence present in or identical to the amino acid stretch which is linked C-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, and
a V_{L} region having an amino acid sequence present in or identical to the amino acid stretch which is linked N-terminally to the sequence GGGGSGGGGSGGGSGGGGS (SEQ ID NO: 137) in the same SEQ ID NO,
   or
a V_{H} region having an amino acid sequence present in or identical to the amino acid stretch which is linked C-terminally to the sequence GGGGSGGGGSGGGGSGGG (SEQ ID NO: 138) in SEQ ID NO: SEQ ID NO: 121, and
a V_{L} region having an amino acid sequence present in or identical to the amino acid stretch which is linked N-terminally to the sequence GGGGSGGGGSGGGGSGGG (SEQ ID NO: 138) in the same SEQ ID NO,
   wherein each of the V_{H} and V_{L} sequences independently can also be a sequence having at least 80% sequence similarity or sequence identity with the SEQ ID NO: indicated. This sequence similarity or sequence similarity can be higher, such as selected from at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, and at least 99%.

Sequence similarities and identities is in this context determined by optimal pairwise global alignment using the Needleman-Wunsch algorithm using the following parameters:
Substitution Matrix: EBLOSUM62
Gap penalty: 10.0
Extend penalty: 0.5

In some embodiments, the CS glycosaminoglycan chain bound by any of the antibodies/variants of the invention has N-acetylgalactosamine (GalNAc) residues with a sulfate group at the C-4 position in >50%, such as >60%, preferably >70%, of the disaccharide repeats of the chain. In addition, the CS chain may in some cases contain 4-O sulfated GalNac and iduronic acid (IdoA). In other embodiments, the CS glycosaminoglycan chain bound by any of the antibodies/variants of the invention has fully 6-O sulfated N-acetylgalactosamine (GalNAc) residues and in some case these CS glycosaminoglycans do not contain 4-O sulfated GalNac and/or iduronic acid (IdoA).

As noted, the antibodies and antibody variants of the present invention exhibit a hitherto unseen affinity for ofCS (see the examples). For some applications preferred antibodies and variants among those disclosed above are those dimers or other multimers, which bind to the ofCS glycosaminoglycan chain with an equilibrium dissociation constant (K_{D}) of <10 nM, such as <5 nM. Whereas for other applications modifications of the antibody fragments with K_{D} up to 40 nM could be preferred, for example in a monomeric monovalent format having a high on rate and a higher off rate resulting in a Kd value of up to 40 nM and a compound that due to its lower molecular weight can more easily penetrate a solid tumor or get more rapidly cleared for radio theranostics purposes.

The antigen for the antibody or the antibody variant described above in the context of the 1^{st} aspect is one where the ofCS glycosaminoglycan chain is attached to a protein core forming a chondroitin sulfate proteoglycan (CSPG) present in secreted form, on a cell membrane or in an extracellular matrix. The CSPG is typically selected from, but not limited to, any one of: Brain natriuretic peptide B, Endothelial cell-specific molecule 1, Sushi repeat-containing protein SRPX, Decorin, Protein AMBP, Biglycan, Bone marrow proteoglycan, Syndecan-4, Amyloid-like protein 2, HLA class II histocompatibility antigen gamma chain, Chondroitin sulfate proteoglycan 4, Agrin, Testican 1-3, Neuropilin, CD44 antigen, Glypican-1-6, Syndecan-1-34, Laminin subunit gamma 2, Carbonic anhydrase 9, Aggrecan, Perlecan, Collagen alpha-1 (XII), Collagen alpha-1 (XV), Collagen alpha-1 (XVIII), Laminin subunit alpha-4, Matrix-remodeling associated protein 5, Nidogen-2, Endocan, and Versican.

As already noted above, the exact format of the antibody of the 1^{st} aspect is not of very high relevance although IgG is preferred for some purposes. With respect to the variant, it can as shown herein have any of the formats from the following non-limiting list: an Fab, and Fab′, and Fab-SH, an F(ab)2, an F(ab′)₂, an ScFv, an Fv fragment, a Heavy chain Ig (such as a llama or camel Ig), a V_{HH} fragment, a dsFV, a minibody, a diabody, a triabody, a kappa body, an IgNAR, a tandAb, a BiTE, and a multispecific antibody. If the antibody is multispecific, a bispecific format is preferred, such as a bispecific antibody binding CD3.

### 2^{nd} Aspect of the Invention and Embodiments Thereof

This aspect relates to conjugate or a fusion protein comprising at least a first and a second moiety, wherein the first moiety is the antibody or the antibody variant according the first aspect of the invention or any embodiment thereof disclosed herein, and the second moiety is a molecule or polypeptide; wherein the first moiety is conjugated or genetically fused to the second moiety, and wherein the second moiety provides or improves the therapeutic and/or diagnostic function of the conjugate or the fusion protein.

To be more specific, this aspect provides for coupling of the antibodies/variants to conjugation of fusion partners that can provide a variety of advantages in detection, purification, or therapeutic activity of the antibodies/variants—or, alternatively, advantages in the therapeutic activity of the conjugation/fusion partners, since the antibodies can act as targeting agents.

For instance the second moiety can be selected from any one of: a toxin or a fragment thereof (useful in cancer therapy), an immune-modulating molecule or a fragment thereof (also of relevance in cancer therapy), a nanoparticle (which can be both useful as a detection label or as a therapeutic moiety), a radionuclide or a radionuclide-containing substance (which also are relevant for both therapy and diagnostic applications), and a label (i.e. a detectable moiety). Also, the second moiety can be a gene therapeutic agent, such as a poly- or oligonucleotide (such as mRNA, for instance mRNA including capped nucleotides) or a poly- or oligonucleotide comprising modified nucleosides. For instance, the modifications can be with any modification of the pentose moiety, sugar/backbone, the backbone, the base moiety as well as introduction of unnatural base pairs. Thus, the sugar modifications can be 2′F RNA, 2′Ome RNA, LNA, FANA, HNA, or 2′MOE, sugar/backbone modifications can be mirror DNA, ribuloNA, TNA, t-PhoNA, or dXNA, backbone modifications can be PS, phNA, PNA, and boranophosphate, base modifications can be C7-modified deaza-adenine, C-7-modified deaza-guanosine, C5-modified cytosine, and C5-modified uridine, where the modifications include addition of hydrogen, Chloride, Fluoride, or Bromide and the unnatural base pairs, UBPs can be dZ-dP, Ds-Px, 5SICSN-aM, dS-dB, Ds-Pa, and TPT3-NaM. For details concerning such modifications, cf. Duffy K et al. (2020), BMC Biology 18: 112 (doi.org/10.1186/s12915-020-00803-6). Also, the fusion/conjugation partner can be an aptamer.

Also, the second moiety can e.g. be another specific antibody or antibody variant, so as to provide for a bi- or multispecific antibody format. For instance, the other moiety could be an anti-CD3 fused to an antibody or antibody variant of the present invention. Another possibility would be to couple the antibodies of the present invention to a molecule comprising a radionuclide. For a review of state-of-the art radionuclides useful in target radionuclide therapy, reference is made to Goldsmith S. J. (2020), Semin Nucl Med 50(1):87-97. (doi: 10.1053/j.semnuclmed.2019.07.006), which is hereby incorporated by reference in its entirety herein. As an alternative, such radionuclides may—instead of being part of a second moiety—be an integral part of an antibody or antibody variant of the invention.

Preferred radionuclides include without limitation Iodine-131, Yttrium-90, and Lutetium-177 (all commonly used in therapy), as well as fluorine-18, gallium-67, krypton-81m, rubidium-82, nitrogen-13, technetium-99m, indium-111, iodine-123, xenon-133, and thallium-201 (which are all commonly used in imaging technologies).

In important embodiments, the second moiety is a toxin, which can be highly useful in therapy. In the event the antibody is targeted exclusively or at least preferentially to tumour tissue, such a toxin can be of any type that will induce cell death (necrotic or, preferably apoptotic cell death), but in most embodiments it is preferably selected from a cytotoxic or cytostatic agent, such as an alkylating agent, an antimetabolite, an anti-microtubule agent such as monomethyl auristatin E (MMAE), a topoisomerase inhibitor such as exetecan, and a cytotoxic antibiotic.

In essence, any cytotoxic or cytostatic agent commonly used for cancer therapy can be used, but the coupling to the antibody/variant expands the field of selection of cytotoxic and cytostatic agents to those that are too toxic for systemic administration (MMAE is an example), but which can be employed because the coupling to the antibody/variant prevents systemic dissemination of the toxin.

The alkylating agents can be nitrogen mustards, nitrosoureas, tetrazines, aziridines, cisplatins and derivatives. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. Further, the alkylating agents also include procarbazine and hexamethylmelamine.

The antimetabolites include anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents include the vinca alkaloids and taxanes, Vinca alkaloids include vincristine, vinblastine, vinorelbine, vindesine, and vinflunine. Taxanes include paclitaxel, docetaxel

Podophyllotoxin is also an anti-microtubule agent and acts in a manner similar to that of vinca alkaloids.

Topoisomerase inhibitors include irinotecan and topotecan, etoposide, doxorubicin, mitoxantrone, teniposide, novobiocin, merbarone, camptothecin, and aclarubicin.

The cytotoxic antibiotics include anthracyclines, bleomycin, mitomycin C, and actinomycin. Important anthracyclines are doxorubicin, daunorubicin, epirubicin, idarubicin, pirarubicin, aclarubicin, and mitoxantrone. Immune checkpoint inhibitors include those that target CTLA4, PD-1, or PD-L1, and include Ipilimumab (targets CTLA-4), Nivolumab (targets PD-1), Pembrolizumab (targets PD-1), Atezolizumab (targets PDL-1), Avelumab (targets PDL-1), Durvalumab (targets PDL-1), and Cemiplimab (targets PD-1). Further, also those inhibitors that exhibit ubiquitin ligase actively, such as CISH (cytokine-inducible SH2-containing protein) and CBL are relevant.

Some conjugates of the 2^{nd} aspect are those where the second moiety is a polypeptide, and wherein the first moiety is genetically fused to the second moiety. Particularly interesting fusion partners of the antibody/variant are cytokines and chemokines; particularly important are interleukin-2 (IL-2), interleukin-7 (IL-7), interleukin-12, granulocyte-macrophage colony-stimulating factor (GM-CSF), tumour necrosis factor (TNF), or TNF-related apoptosis-inducing ligand (TRAIL).

### 3^{rd} Aspect of the Invention and Embodiments Thereof

This aspect relates to a polypeptide comprising i) a first polypeptide domain being a transmembrane domain and an endodomain of a chimeric antigen receptor (CAR), and ii) a second polypeptide domain being the antibody or the antibody variant according to the 1^{st} aspect of the invention or any embodiment thereof disclosed herein. Such a fusion protein has specific relevance for production of a CAR-T cell, which in turn is useful in therapy.

Details concerning construction of fusions between antibodies and CAR can i.a. be found in Larson, R C et al. (2021), Nature Reviews Cancer 21: 145-161 and Feins S et et al. (2019), Hematol 94(S1): S3-S9.

### 4^{th} Aspect of the Invention and Embodiments Thereof

This aspect relates to CAR-T cell comprising the polypeptide of 3^{rd} aspect of the invention or any embodiment thereof disclosed herein.

Details concerning construction of CAR-T cells can i.a. be found in Larson, R C et al. (2021), Nature Reviews Cancer 21: 145-161 and Feins S et et al. (2019), Hematol 94(S1): S3-S9.

### 5^{th} Aspect of the Invention and Embodiments Thereof

This aspect relates to an isolated nucleic acid molecule comprising a nucleotide sequence encoding the antibody or the antibody variant of the 1^{st} aspect of the invention or any embodiment thereof disclosed herein, the conjugate or the fusion protein of the 2^{nd} aspect of the invention or any embodiment thereof disclosed herein, or the polypeptide of the 3^{rd} aspect of the invention or any embodiment thereof disclosed herein.

The antibodies/variants disclosed herein are primarily derived from human B-cells, meaning that the genetic material relevant for the antibody production in a recombinant system is suitable for expression in human cells. However, codons can be changed according to the genetic code, and thereby expression can be optimized for recombinant production in virtually any host cell.

Further, the skilled person will be able to introduce the essential antigen binding regions (Fvs or CDRs) into any vector backbone, which encodes the desired antibody or antibody variant format.

### 6^{th} Aspect of the Invention and Embodiments Thereof

This aspect relates to a vector comprising the isolated nucleic acid molecule of the 5^{th} aspect of the invention or any embodiment thereof disclosed herein, such as an expression vector or a cloning vector. In the event the vector is an expression vector, it will include the necessary genetic elements for this purpose:

One preferred vector disclosed herein comprises in operable linkage and in the 5′-3′ direction, an expression control region comprising an enhancer/promoter for driving expression of the nucleic acid fragment of the 5^{th} aspect, optionally a signal peptide coding sequence, a nucleotide sequence of the 5^{th} aspect, and optionally a terminator. Hence, such a vector constitutes an expression vector useful for effecting production in cells of the antibody, variant or polypeptide of the 1^{st}, where relevant 2^{nd}, and 3^{rd} aspects (jointly termed “polypeptides of the invention”). Since the polypeptides of the invention are mammalian of origin, recombinant production is conveniently carried out in eukaryotic host cells, so here it is preferred that the expression control region drives expression in eukaryotic cells (such as plant, fungal, insect, and mammalian cells). However, many antibody variants can be produced in bacteria, so here it is preferred that the expression control region drives expression in eukaryotic cells prokaryotic cell such as a bacterium, e.g., in E coli.

The vector may as indicated further comprise a sequence encoding a signal peptide, which may provide for secretion or membrane integration of the expression product from said vector.

At any rate, certain vectors disclosed herein are capable of autonomous replication.

Also, the vector disclosed herein may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in initial screening for expression efficiency.

Typically, the vector disclosed herein is selected from the group consisting of a virus, a bacteriophage, a plasmid, a minichromosome, and a cosmid.

A more detailed discussion of vectors disclosed herein is provided in the following:

Polypeptides disclosed herein may be encoded by a nucleic acid of the 5^{th} aspect comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or a fusion partner that stimulates the immune system, such as a cytokine or active fragment thereof. Useful vectors encoding such fusion proteins include pIN vectors, vectors encoding a stretch of histidinyl residues, and pGEX vectors, for use in generating glutathione S-transferase (GST) soluble fusion proteins for later purification and separation or cleavage.

Vectors disclosed herein may be used in a host cell to produce a polypeptide disclosed herein that may subsequently be purified for administration to a subject.

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous”. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein.

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRα, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), αI-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumour virus)—Glucocorticoids; β-Interferon—poly(rl)x/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2Kb—Interferon; HSP70—E1A/SV40 Large T Antigen; Proliferin—Phorbol Ester/TPA; Tumour Necrosis Factor—PMA; and Thyroid Stimulating Hormonea Gene—Thyroid Hormone.

Also contemplated as useful in the present invention are the dectin-1 and dectin-2 promoters. Additionally, any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide disclosed herein is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments disclosed herein, the use of internal ribosome entry sites (IRES) elements is to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 2. Multiple Cloning Sites

Vectors can include a multiple cloning site (MCS), which is a nucleic acid region that contains multiple restriction enzyme sites, any of which can be used in conjunction with standard recombinant technology to digest the vector. Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

### 3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression.

### 4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport. Consequently, the corresponding encoded RNA fragment preferably comprises a poly(A) tail.

### 6. Origins of Replication

In order to propagate a vector in a host cell, it may contain one or more origins of replication sites (often termed “on”), which is a specific nucleic acid sequence at which replication is initiated. Alternatively, an autonomously replicating sequence (ARS) can be employed if the host cell is yeast.

### 7. Selectable and Screenable Markers

In certain embodiments disclosed herein, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein disclosed herein. Further examples of selectable and screenable markers are well known to one of skill in the art.

### 7^{th} Aspect of the Invention and Embodiments Thereof

This aspect relates to a host cell comprising or transformed with the vector of the 6^{th} aspect of the invention or any embodiment thereof disclosed herein.

Transformed cells disclosed herein are useful as organisms for producing the polypeptide or the chimeric polypeptide of the invention, but also as simple “containers” of nucleic acids and vectors disclosed herein.

Certain transformed cells disclosed herein are capable of replicating the nucleic acid fragment of the 5^{th} aspect. Preferred transformed cells disclosed herein are capable of expressing the nucleic acid of the 5^{th} aspect.

For recombinant production it is convenient, but not a prerequisite that the transformed cell according is prokaryotic, such as a bacterium, but generally both prokaryotic cells and eukaryotic cells may be used, cf. the considerations above.

Suitable prokaryotic cells are bacterial cells selected from the group consisting of Escherichia (such as E. coli.), Bacillus [e.g., Bacillus subtilis], and Salmonella. A preferred E. coli cell is Shuffle cells or similar coli strains that allows for disulfide bonds to form in the cytoplasm enabling the scFV fold.

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian, such as human, cell.

For production purposes, it is advantageous that the transformed cell disclosed herein is stably transformed by having the nucleic acid defined above for option i) stably integrated into its genome, and in certain embodiments it is also preferred that the transformed cell secretes or carries on its surface the polypeptide disclosed herein, since this facilitates recovery of the polypeptides produced. A particular version of this embodiment is one where the transformed cell is a bacterium and secretion of the polypeptide disclosed herein is into the periplasmic space.

An interesting production system is the use of plants. For instance, proteins can be produced at low cost in plants using an Agrobacterium transfection system to genetically modify plants to express genes that encode the protein of interest. Commercially available platforms are those provided by iBio CMO LLC (8800 HSC Pkwy, Bryan, TX 77807, USA) and iBio, Inc (9 Innovation Way, Suite 100, Newark, DE 19711, USA) and disclosed in e.g. EP 2 853 599, EP 1 769 068, and EP 2 192 172. Hence, in such systems the vector is an Agrobacterium vector or other vector suitable for transfection of plants.

As noted above, stably transformed cells are preferred—these i.a. allows that cell lines comprised of transformed cells as defined herein may be established—such cell lines are particularly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli Shuffle); members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe). As mentioned above, the nucleic acid sequence of the present invention can be appropriately codon optimized to facilitate effective expression from each of the transformed cells disclosed herein.

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials or from other depository institutions such as Deutsche Sammlung von Micrroorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JMI 09, and KC8, as well as a number of commercially available bacterial hosts such as SURE® Competent Cells and SOLOP ACK™ Gold Cells (STRATAGENE®, La Jolla, CA). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, HEK293, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above-described host cells to maintain them and to permit replication of a vector. Also understood and known are techniques and conditions that would allow large-scale production of vectors, as well as production of the nucleic acids encoded by vectors and their cognate polypeptides, proteins, or peptides.

Numerous expression systems exist that comprise at least a part or all of the components discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the dis'losed expression systems disclosed herein, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### 8^{th} Aspect of the Invention and Embodiments Thereof

This aspect relates to a method for producing the antibody or the antibody variant according to the 1^{st} aspect of the invention or any embodiment thereof disclosed herein, the conjugate or the fusion protein of the 2^{nd} aspect of the invention or any embodiment thereof disclosed herein, or the polypeptide of the 3^{rd} aspect of the invention or any embodiment thereof disclosed herein, the method comprising the steps of: transfecting or transforming a host cell with the vector of the 6^{th} aspect of the invention or any embodiment thereof disclosed herein, expressing the nucleotide sequence according to the 5th aspect of the invention or any embodiment thereof disclosed herein, and isolating the antibody or the antibody variant, the conjugate or the fusion protein, or the polypeptide.

In general, the disclosure presented above under the 6^{th} and 7^{th} aspects apply mutatis mutandis the 8^{th} aspect.

### 9^{th} Aspect of the Invention and Embodiments Thereof

This aspect relates to a pharmaceutical composition comprising the antibody or the antibody variant of the 1^{st} aspect of the invention or any embodiment thereof disclosed herein, the conjugate or the fusion protein of the 2^{nd} aspect of the invention or any embodiment thereof disclosed herein, or the CAR-T cell of the 4^{th} aspect of the invention or any embodiment thereof disclosed herein, and a pharmaceutically acceptable carrier, vehicle or diluent. This aspect thus relates generally to a pharmaceutical composition comprising antibodies or variants thereof. For details on such formulations, including the choice carriers, vehicles, diluents, and excipients, reference is generally made to state-of the art technology for formulation of antibodies and their derivatives, see below.

In some embodiments, the pharmaceutical composition can contain at least 0.1% by weight of the antibodies/antibody variants, such as at least 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, or more by weight of antibodies/antibody variants. In other embodiments, for example, antibodies/antibody variants can constitute between about 2% to about 75% of the weight of the composition, between about 25% to about 60%, between about 30% to about 50%, or any range therein.

The pharmaceutical composition further includes one or more additional ingredients. A pharmaceutically acceptable carrier can be a carrier approved by a regulatory agency of the Federal or a state government, or listed in the U.S. Pharmacopeia, European Pharmacopeia or other generally recognized Pharmacopeia for use in animals, and more particularly in humans.

The preparation of a pharmaceutical composition having the antibodies or other molecules as described herein as active ingredient are known to those of skill in the art in light of the present disclosure, as exemplified by Remington's Pharmaceutical Sciences, 18th Ed., 1990, incorporated herein by reference. Moreover, for animal (including human) administration, it is understood that preparations should meet sterility, pyrogenicity, general safety, and purity standards as required by FDA Office of Biological Standards or as required by the European Medicines Agency (EMA).

Pharmaceutically acceptable carriers include liquid, semi-solid, i.e., pastes, or solid carriers. Examples of carriers or diluents include fats, oils, water, saline solutions, lipids, liposomes, resins, binders, fillers, and the like, or combinations thereof. The pharmaceutically acceptable carrier can include aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatine), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc), gels, binders, excipients, disintegration agents, lubricants, sweetening agents, flavouring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in administrable composition for use in practicing the methods is appropriate. The pH and exact concentration of the various components in a pharmaceutical composition are adjusted according to well-known parameters. In accordance with certain aspects of the present disclosure, the composition can be combined with the carrier in any convenient and practical manner, i.e., by solution, suspension, emulsification, admixture, encapsulation, absorption, grinding, and the like. Such procedures are routine for those skilled in the art.

In some embodiments, a pharmaceutically acceptable carrier can be an aqueous pH buffered solution. Examples include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In some embodiments, pharmaceutically acceptable carriers can be sterile liquids, such as water and oils, including those of petroleum, animal, vegetable or synthetic origin, such as peanut oil, soybean oil, mineral oil, sesame oil and the like. Water can be a carrier, particularly when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol, polysorbate-80 and the like. The composition can also contain minor amounts of wetting or emulsifying agents, or pH buffering agents. These compositions can take the form of solutions, suspensions, emulsion, tablets, pills, capsules, powders, sustained-release formulations and the like.

Certain embodiments of the present disclosure can have different types of carriers depending on whether it is to be administered in solid, liquid, or aerosol form, and whether it needs to be sterile for the route of administration, such as injection. The compositions can be formulated for administration intravenously, intradermally, transdermally, intrathecally, intraarterially, intraperitoneally, intranasally, intravaginally, intrarectally, intramuscularly, subcutaneously, mucosally, intratumoral, peritumoral, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). Typically, such compositions can be prepared as either liquid solutions or suspensions; solid forms suitable for use to prepare solutions or suspensions upon the addition of a liquid prior to injection can also be prepared; and, the preparations can also be emulsified.

The antibodies/antibody variants can be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, 2-ethylamino ethanol, histidine, or procaine.

In further embodiments, provided herein are pharmaceutical compositions comprising lipid. Lipids broadly include a class of substances that are characteristically insoluble in water and extractable with an organic solvent. Examples include compounds that contain long-chain aliphatic hydrocarbons and their derivatives. A lipid can be naturally occurring or synthetic (i.e., designed or produced by man). A lipid can be a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Compounds other than those specifically described herein that are understood by one of skill in the art as lipids can also be used.

The skilled in the art would be familiar with the range of techniques that can be employed for dispersing a composition in a lipid vehicle. For example, antibodies can be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of active ingredient in each therapeutically useful composition can be prepared in such a way that a suitable dosage will be obtained in any given unit dose of the compound. Factors, such as solubility, bioavailability, biological half-life, route of administration, product shelf life, as well as other pharmacological considerations, can be contemplated by one skilled in the art of preparing such pharmaceutical formulations, and as such, a variety of dosages and treatment regimens may be desirable.

A “unit dose” or “unit dosage” refers to physically discrete units suitable for use in a subject, each unit containing a predetermined quantity of the pharmaceutical composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. In other non-limiting examples, a dose can have from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above.

The practitioner responsible for administration will, in any event, determine the concentration of active ingredient(s) in a composition and appropriate dose(s) for the individual subject.

As a person of ordinary skill in the art would understand, the compositions described herein are not limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy varies according to the type of use and mode of administration, as well as the particularized requirements of individual subjects. The actual dosage amount of a composition administered to an animal patient, including a human patient, can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount can vary according to the response of the subject. The practitioner responsible for administration will, in any event, determine the concentration of active ingredient(s) in a composition and appropriate dose(s) for the individual subject.

### 10^{th} Aspect of the Invention and Embodiments Thereof

This aspect relates to use of the antibody or the antibody variant of the 1^{st} aspect of the invention or any embodiment thereof disclosed herein, or the conjugate or the fusion protein of the 2^{nd} aspect of the invention or any embodiment thereof disclosed herein, for in vitro detection and/or isolation of cancer cells, such as cancer cells derived from a subject, such as circulating tumour cells, and for minimal residual disease detection in haematological tumors.

In this context, reference is made to the detailed disclosure in the Examples as well as in the context of the 14^{th} and 15^{th} aspects of the invention as well as any embodiments thereof disclosed herein.

However, generally the cancer cells are derived from a cancer selected from an epithelial tumour, a non-epithelial tumour (such as a hematological cancer), and a mixed tumour. The epithelial tumour can be any of a carcinoma or an adenocarcinoma, and the non-epithelial tumour or mixed tumour can be any of a liposarcoma, a fibrosarcoma, a chondrosarcoma, an osteosarcoma, a leiomyosarcoma, a rhabomyosarcoma, a glioma, a neuroblastoma, a medullablastoma, a malignant melanoma, a malignant meningioma, a neurofibrosarcoma, a leukemia, a myeloproleferative disorder, a lymphoma (such as a B-cell or T-cell lymphoma), a hemangiosarcoma, a Kaposi's sarcoma, a malignant teratoma, a dysgerminoma, a seminoma, a mesothelioma, or a choriosarcomamelanoma. Also, the anatomic location of the cancer can vary: it can be a cancer of the lung, the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the prostate gland, the ovaries, the uterus, the uterine cervix, the spleen, bone, or cartilage.

### 11^{th} Aspect of the Invention and Embodiments Thereof

This aspect relates to the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein, for use as a medicament.

As appears from the examples below, the antibodies and antibody variants of the invention target a variety of malignant tumours, thus rendering the antibodies and antibody variants as well as products derived thereof highly relevant in methods for treatment of malignancies. Typically, the medicament will be used for treatment of mammals, in particular humans, but also domestic animals such as canines, felines, and equines.

### 12^{th} Aspect of the Invention and Embodiments Thereof

This aspect relates the antibody or the antibody variant of the 1^{st} aspect of the invention or any embodiment thereof disclosed herein, the conjugate or the fusion protein of the 2^{nd} aspect of the invention or any embodiment thereof disclosed herein, or the pharmaceutical composition of the 9^{th} aspect of the invention or any embodiment thereof disclosed herein, for use in diagnosing cancer in a subject.

Reference is made to the more detailed disclosure below concerning methods for diagnosis, but also as is the case for the therapeutic aspects of the invention, the subjects which are diagnosed according to the invention are primarily mammals, in particular humans, but also domestic animals such as canines, felines, and equines.

### 13^{th} Aspect of the Invention and Embodiments Thereof

This aspect relates to the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein, for use in treating cancer or treating a condition involving expression, such as inappropriate expression, of ofCS, such as in a condition selected from the group consisting of arthritis, arthrosis, multiple sclerosis, healing after neural damage, cartilage repair, wound healing, and psoriasis, in a subject in need thereof. In addition, this aspect also relates to uses where the placenta is targeted for delivery of therapeutic agents, e.g. when treating preeclampsia—this latter approach also applies to the 15^{th} aspect of the invention.

Apart from the fact that cancers have been demonstrated to (over)express ofCS, the same is true for the further conditions mentioned above, and since the target (ofCS) is unlikely to be expressed in any appreciable amount in healthy tissues, targeting thereof is contemplated to be a highly safe approach.

As appears from the examples below, the antibodies and antibody variants of the invention target a variety of malignant tumours, thus rendering the antibodies and antibody variants as well as products derived thereof highly relevant for use in methods for treatment of malignancies. Typically, the treatment is of mammals, in particular humans, but also domestic animals such as canines, felines, and equines.

With respect to the cancer treated, the cancer cells are derived from a cancer selected from an epithelial tumour, a non-epithelial tumour (such as a hematological cancer), and a mixed tumour. The epithelial tumour can be any of a carcinoma or an adenocarcinoma, and the non-epithelial tumour or mixed tumour can be any of a liposarcoma, a fibrosarcoma, a chondrosarcoma, an osteosarcoma, a leiomyosarcoma, a rhabomyosarcoma, a glioma, a neuroblastoma, a medullablastoma, a malignant melanoma, a malignant meningioma, a neurofibrosarcoma, a leukemia, a myeloproleferative disorder, a lymphoma (such as a B-cell or T-cell lymphoma), a hemangiosarcoma, a Kaposi's sarcoma, a malignant teratoma, a dysgerminoma, a seminoma, a mesothelioma, or a choriosarcomamelanoma. Also, the anatomic location of the cancer can vary: it can be a cancer of the lung, the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the prostate gland, the ovaries, the uterus, the uterine cervix, the spleen, bone, or cartilage.

### 14^{th} Aspect of the Invention and Embodiments Thereof

This important aspect relates to a method of treating a cancer or treating a condition involving expression, such as inappropriate expression, of ofCS, such as in a condition selected from the group consisting of arthritis, arthrosis, multiple sclerosis, healing after neural damage, cartilage repair, wound healing, and psoriasis, or for delivering a therapeutic to the placenta, in a subject in need thereof, the method comprising administering to the subject a therapeutically effective amount of the antibody or the antibody variant of the 1^{st} aspect of the invention or any embodiment thereof disclosed herein, the conjugate or the fusion protein of the 2^{nd} aspect of the invention or any embodiment thereof disclosed herein, the CAR-T cell of the 4^{th} aspect of the invention or any embodiment thereof disclosed herein, or the pharmaceutical composition of the 9^{th} aspect of the invention or any embodiment thereof disclosed herein.

Apart from the fact that cancers have been demonstrated to (over)express ofCS, the same is true for the further conditions mentioned above, and since the target (ofCS) is unlikely to be expressed in any appreciable amount in healthy tissues, targeting thereof is contemplated to be a highly safe approach.

With respect to the cancer treated, the cancer cells are derived from a cancer selected from an epithelial tumour, a non-epithelial tumour (such as a hematological cancer), and a mixed tumour. The epithelial tumour can be any of a carcinoma or an adenocarcinoma, and the non-epithelial tumour or mixed tumour can be any of a liposarcoma, a fibrosarcoma, a chondrosarcoma, an osteosarcoma, a leiomyosarcoma, a rhabomyosarcoma, a glioma, a neuroblastoma, a medullablastoma, a malignant melanoma, a malignant meningioma, a neurofibrosarcoma, a leukemia, a myeloproleferative disorder, a lymphoma (such as a B-cell or T-cell lymphoma), a hemangiosarcoma, a Kaposi's sarcoma, a malignant teratoma, a dysgerminoma, a seminoma, a mesothelioma, or a choriosarcomamelanoma. Also, the anatomic location of the cancer can vary: it can be a cancer of the lung, the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the prostate gland, the ovaries, the uterus, the uterine cervix, the spleen, bone, or cartilage.

Various delivery systems are also known and can be used to administer the antibodies and antibody variants or the related pharmaceutical compositions or CAR-T cells, such as encapsulation in liposomes, microparticles, and microcapsules.

The methods of administration as provided herein include, but are not limited to, injection, as by parenteral administration (e.g., intradermal, intramuscular, intraperitoneal, intravenous and subcutaneous), epidural, and mucosal (e.g., intranasal and oral routes). In some embodiments, the antibodies, other molecules, or pharmaceutical compositions provided herein are administered intramuscularly, intravenously, subcutaneously, intravenously, intraperitoneally, orally, intramuscularly, subcutaneously, intracavity, transdermally, or dermally. The compositions can be administered by any convenient route, for example, by infusion or bolus injection, by absorption through epithelial or mucocutaneous linings (e.g., oral mucosa, rectal and intestinal mucosa, etc.) and can be administered together with other biologically active agents. Administration can be systemic or local. In addition, pulmonary administration can also be employed, e.g., by use of an inhaler or nebulizer, and formulation with an aerosolizing agent. In some embodiments, administration is local to the area in need of treatment, e.g., by local infusion, by injection, or by means of an implant, said implant being of a porous, non-porous, or gelatinous material, including membranes, such as sialastic membranes, or fibers. In some embodiments, when administering antibodies or antibody variants as described herein, care is taken to use materials to which the antibodies or antibody variants do not absorb.

In some embodiments, antibodies or antibody variants provided herein are formulated in liposomes for targeted delivery. Liposomes are vesicles comprised of concentrically ordered phospholipid bilayers which encapsulate an aqueous phase. Liposomes typically have various types of lipids, phospholipids, and/or surfactants. The components of liposomes are arranged in a bilayer configuration, similar to the lipid arrangement of biological membranes. Liposomes can be useful delivery vehicles due, in part, to their biocompatibility, low immunogenicity, and low toxicity. Methods for preparation of liposomes are known in the art.

Provided herein are also methods of treating a cancer patient by administering a unit dose to the patient the antibodies or antibody variants, CAR-T cell or pharmaceutical composition disclosed as part of the invention herein. A unit dose refers to physically discrete units suitable as unitary dosage for the subject, each unit containing a predetermined quantity of active material calculated to produce the desired therapeutic effect in association with the required diluent, i.e., carrier, or vehicle.

Administration is made in a manner compatible with the dosage formulation, and in a'therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual subject. However, suitable dosage ranges for systemic application are disclosed herein and depend on the route of administration. Suitable regimes for initial and booster administration are also contemplated and typically include by an initial administration followed by repeated doses at one or more hour-intervals by a subsequent injection or other administration. Exemplary multiple administrations are described above and are useful to maintain continuously high serum and tissue levels of polypeptide or antibody. Alternatively, continuous intravenous infusion sufficient to maintain concentrations in the blood in the ranges specified for in vivo therapies are contemplated.

A therapeutically effective amount is a predetermined amount calculated to achieve the desired effect. Generally, the dosage will vary with age of, condition of, sex of, and extent of the disease in the patient and can be determined by one of skill in the art. The dosage can be adjusted by the individual physician in the event of any complication.

The precise dose to be employed in an administered formulation will also depend on the route of administration, and the seriousness of the condition, and should be decided according to the judgment of the practitioner and each patient's circumstances. Effective doses can be extrapolated from dose-response curves derived from in vitro or animal model test systems; dosages are detailed above under the discussion of the pharmaceutical compositions of the present invention. Typically, the dosage administered to a patient is typically 0.01 mg/kg to 100 mg/kg of the patient's body weight. In some embodiments, the dosage administered to a patient is between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. In particular, the dosage administered to a patient can be 0.2 mg/kg, 0.3 mg/kg, 1 mg/kg, 3 mg/kg, 6 mg/kg or 10 mg/kg. A dose as low as 0.01 mg/kg is predicted to show appreciable pharmacodynamic effects. Dose levels of 0.10-1 mg/kg are predicted to be most appropriate. Higher doses (e.g., 1-30 mg/kg) can also be expected to be active. Generally, human and humanized antibodies have a longer half-life within the human body than antibodies from other species due to the immune response to the foreign polypeptides. Thus, lower dosages of human antibodies and less frequent administration can be practiced. Further, the dosage and frequency of administration of antibodies or antibody variants provided herein can be reduced by enhancing uptake and tissue penetration of the antibodies by modifications such as, for example, lipidation.

In yet another embodiment, delivery can be made in a controlled release or sustained release system. Any technique known to one of skill in the art can be used to produce sustained release formulations having one or more antibodies, molecules, or pharmaceutical compositions provided herein. In one embodiment, a pump can be used in a controlled release system. In another embodiment, polymeric materials can be used to achieve controlled release of antibodies.

Examples of polymers that can be used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In yet another embodiment, a controlled release system can be placed in proximity of the therapeutic target (e.g., the lungs), thus requiring only a fraction of the systemic dose. In another embodiment, polymeric compositions useful as controlled release implants are used according to Dunn et al. (see U.S. Pat. No. 5,945,155. Based upon the therapeutic effect of the in situ-controlled release of the bioactive material from the polymer system, the implantation can generally occur anywhere within the body of the patient in need of therapeutic treatment.

In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (see U.S. Pat. No. 5,888,533). Any technique known to one of skill in the art can be used to produce sustained release formulations comprising one or more therapeutic agents provided herein.

Treatment of a subject can include a single treatment or a series of treatments. It is contemplated that administration can be made systemically or locally to treat disease, such as to inhibit tumour cell growth or to kill cancer cells in cancer patients with locally advanced or metastatic cancers. Administration can be made intravenously, intrathecally, and/or intraperitoneally. Further, the antibodies and variants can be administered alone or in combination with anti-proliferative drugs. In this context, use can be made of any of the fusion and conjugation partners described above in the context of fusion partners to the antibodies and antibody variants of the present invention. In one embodiment, they are administered to reduce the cancer load in the patient prior to surgery or other procedures. Alternatively, they can be administered after surgery to ensure that any remaining cancer (e.g., cancer that the surgery failed to eliminate) does not survive. In some embodiments, they can be administered after the regression of primary cancer to prevent metastasis.

So, the antibodies, antibody variants, pharmaceutical composition and CAR-T of the present invention can be administered in combination with a second therapy. In some embodiments, the second therapy is an anti-cancer or anti-hyperproliferative therapy.

In some embodiments, the compositions and methods that include administration of the antibodies or antibody variants or CAR-T provided herein, when used in combination with another anti-cancer or anti-hyperproliferative therapy, can enhance the therapeutic potency of the other anti-cancer or anti-hyperproliferative therapy. Accordingly, methods and compositions described herein can be provided in combination with a second therapy to achieve the desired effect, such as killing of a cancer cell, inhibition of cellular hyperproliferation, and/or inhibition of cancer metastasis.

In some embodiments, the second therapy has a direct cytotoxic effect, such as a chemotherapy, a targeted therapy, a cryotherapy, a hyperthermia therapy, a photodynamic therapy, a high intensity focused ultrasound (HIFU) therapy, a radiotherapy, or a surgical therapy. The targeted therapy can be a biological targeted therapy or a small molecule targeted therapy. In other embodiments, the second therapy does not have a direct cytotoxic effect. For example, the second therapy may be an agent that upregulates the immune system without having a direct cytotoxic effect.

Provided herein are methods that include administration of the antibodies, antibody variants, pharmaceutical composition and CAR-T of the present invention in combination with a second or additional therapy. Administration can be made before, during, after, or in various combinations relative to the second anti-cancer therapy. The administrations can be in intervals ranging from concurrently to minutes to days to weeks. In some embodiments where the antibodies, antibody variants, pharmaceutical composition and CAR-T described herein are provided to a patient separately from a second anti-cancer agent, one would generally ensure that a significant period of time did not expire between the time of each delivery, such that the two compounds would still be able to exert an advantageously combined effect on the patient. In such instances, it is contemplated that one can provide a patient with the antibodies, antibody variants, pharmaceutical composition and CAR-T provided herein, and the second anti-cancer therapy within about 12 to 24 or 72 h of each other and, more particularly, within about 6-12 h of each other. In some situations, the time period for treatment can be extended significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent can be given on any day of day 1 to day 90 (this such range includes intervening days) or any combination thereof, and another agent is given on any day of day 1 to day 90 (this such range includes intervening days) or any combination thereof. Within a single day (24-hour period), the patient can be given one or multiple administrations of the agent(s). Moreover, after a course of treatment, it is contemplated that there is a period of time at which no anti-cancer treatment is administered. This time period can last 1-7 days, and/or 1-5 weeks, and/or 1-12 months or more (this such range includes intervening days), depending on the condition of the patient, such as their prognosis, strength, health, etc. The treatment cycles can be repeated as necessary.

### 15^{th} Aspect of the Invention and Embodiments Thereof

This aspect relates to a method for detection of ofCS in a sample, the method comprising
1) contacting said sample with an antibody or antibody variant of the 1^{st} aspect of the invention or any embodiment thereof disclosed herein, to allow the antibody or antibody variant to specifically bind ofCS, subsequently contacting the antibody or antibody variant and any ofCS bound thereby with an agent, which is capable of binding ofCS, to allow said agent to bind ofCS, or
2) contacting said sample with an agent, which is capable of binding ofCSPG, to allow the agent to bind ofCS, subsequently contacting the agent and any ofCS bound thereby with an antibody or antibody variant of the 1^{st} aspect of the invention or any embodiment thereof disclosed herein, to allow the antibody to bind ofCS,
and detecting quantitatively or qualitatively the presence of complexes comprising the ofCS, the antibody or antibody variant, and the agent, wherein the agent and the antibody or antibody variant are either competitive or non-competitive for their binding to ofCS.

One very attractive feature of the antibodies identified as part of the present invention is that they not all compete for binding to the same epitope on ofCS. This provides for the use the antibodies in a setup, where one can function as capture antibody—typically coupled to a solid or semi-solid surface—and the other as a detection/detectable antibody, without the antibodies being able to displace each other and cause inaccuracies in an assay. This facilitates direct assays. Other antibodies are competing for binding to ofCS, thus opening for competitive assay formats.

The sample is a sample typically comprises or consists of cells and/or tissue, preferably a bodily fluid, such as blood, urine, saliva, CNS fluid, and lymph; feces; and a biopsy. The sample is typically diluted in a suitable sampling liquid, such as a sampling buffer.

The agent in option 1 is in one embodiment an antibody or antibody fragment of the 1^{st} aspect of the invention and any embodiment thereof, VAR2CSA (SEQ ID NO: 135) or a CS binding fragment thereof such as a protein consisting of or comprising SEQ ID NO: 136, an antibody that binds chondroitin sulfate, an antibody that binds the protein core of a proteoglycan (a particularly preferred possibility), or a glycosaminoglycan staining dye.

In option 2, the agent is typically VAR2CSA (SEQ ID NO: 135) or a CSA binding fragment thereof (such as SEQ ID NO: 136) or an antibody that binds the protein core of a proteoglycan.

As mentioned above, it is convenient that the agent or antibody or antibody variant is coupled to a solid or semi-solid phase.

Also, detection of the complex is preferably facilitated by the antibody or the agent being labelled with a detectable moiety or by adding a detectable agent, which binds the antibody or agent. It is however also possible to detect the complex by mass spectrotrophotometric methods, which are well known in the art.

Any conventional assay format can be used. Enzyme-linked immunosorbent assay ELISA, radio immuno assay (RIA), real-time immunoquantitative PCR (iqPCR), microarrays using flourogenic reporters, electrochemilumiscent tag assays, and plasmon resonance formats are all useful.

The latter (plasmon resonance) only requires binding between antibody/antibody variant and target, and hence is a very simple assay format, which only requires the presence of the antibody or variant of the invention as capture agent for ofCS. So, in essence, this assay is limited to contacting an antibody or antibody variant of the invention with the sample and then measuring the interaction via plasmon resonance technology.

### 16^{th} Aspect of the Invention and Embodiments Thereof

This aspect relates to a detection kit comprising an antibody of the 1^{st} aspect of the invention or any embodiment thereof disclosed herein, an agent as defined in the 14^{th} aspect of the invention or any embodiment thereof disclosed herein, and a reaction vessel, and optionally also a labelled agent, which binds the antibody or agent.

Embodiments of this aspect include any conventional assay components—i.e., the reaction vessel can be an ELISA plate, a hollow fiber device, a lateral flow device, a dipstick, a microarray; such assay vessels are known to the person of skill in the art.

Further components of such a kit can be various detection means (fluorescent or luminescent probes, radiolabels, colouring agents etc.) as well as reaction buffers, wash solutions etc.

### 17^{th} Aspect of the Invention and Embodiments Thereof

This aspect relates to a method for providing nucleic acids encoding the antibody or the antibody variant of the 1^{st} aspect of the invention or any embodiment thereof disclosed herein, comprising screening a library comprising antigen-binding fragments of antibodies, wherein the library is comprised of display agents, which are selected from cells, virus, and phage comprising antibody-encoding nucleic acid fragments, for binding of the display agents to a capture agent, wherein the capture agent consists essentially of purified ofCS or ofCS-decorated protein, and subsequently isolating antibody coding nucleic fragments from the capture agent-binding display agents, and optionally sequencing the isolated nucleic acid fragments.

Preferred display agents are selected from yeast cells (when utilising a yeast display technology for antibody identification) or phages selected from the phages M3, fd, T4, T7 and A (when utilising phage display technology for antibody identification).

At any rate, the the antigen-binding fragments of antibodies are typically in the format of scFV, F(ab), F(ab′)₂, and F(ab′). In some embodiments, the antigen binding fragments contain synthetic CDRs, semi-synthetic CDRs, or CDRs derived from a human antibody library.

Related to the 17^{th} aspect, and based on the realization that ofCS-specific antibodies can be identified and isolated via phage display, the inventors have also realized that aptamer libraries can be screened for binders of ofCS in essentially the same way as the screening of a phage display library. Hence, also such a method for identification and isolation of ofCS binding aptamers is part of the present invention, as are aptamers that share the binding characteristics of the antibodies of the present invention.

### 18^{th} Aspect of the Invention and Embodiments Thereof

This aspect relates to a method of producing an antibody or antibody variant of the 1^{st} aspect of the invention or any embodiment thereof disclosed herein, comprising expressing an expression vector comprising a nucleic acid fragment obtained according to the method of the 16^{th} aspect of the invention or any embodiment thereof disclosed herein, in a host cell in a culture and subsequently isolating the expression product from the culture.

The method of the 17^{th} aspect normally provides as the main output the identification of the variable domains of an antibody, thus allowing the person of skill in the art to incorporate the variable domains (or, if desired, the CDRs) into the framework of a desired format such as an IgG or any other antibody/antibody variant format such as those discussed herein, which can be obtained by recombinant expression of a coding sequence in a host cell.

### BIOLOGICAL SEQUENCES

The amino acid sequences referred to in the present specification and claims are the following:

F1 (SEQ ID NO: 1)
DIVMTQSPDSLAVSLGERATINCKSSQSVLGSSNNKNYLAWYQQKPGQPPKLLIYWASTRESGVPDRFS
GSGSGTDFTLTISSLQAEDVAVYYCQQYYRSPYTFGQGTKVEIKGGGGSGGGGSGGGSGGGGSEVQLL
ESGGGLVQPGGSLRLSCAASGFTFSDYAMSWVRQAPGKGLEWVSRISGSGGQTKYADSVKGRFTISRD
NSKNTLYLQMNSLRAEDTAVYYCARGHSSSWLRGH FQHWGQGTLVTVSS
F1-CDR-L1 (SEQ ID NO: 2)
KSSQSVLGSSNNKNYLA
F1-CDR-L2 (SEQ ID NO: 3)
WASTRES
F1-CDR-L3 (SEQ ID NO: 4)
QQYYRSPYT
F1-CDR-H1 (SEQ ID NO: 5)
GFTFSDYAMS
F1-CDR-H2 (SEQ ID NO: 6)
RISGSGGQTKYADSVKG
F1-CDR-H3 (SEQ ID NO: 7)
GHSSSWLRGHFQH
F2 (SEQ ID NO: 8)
DIVMTQSPDSLAVSLGERATINCKSSQSVLRSGNNKNRLAWYQQKPGQPPKLLIYDASTRESGVPDRFS
GSGSGTDFTLTISSLQAEDVAVYYCQQQYNYPYTFGQGTKVEIKGGGGSGGGGSGGGSGGGGSEVQLL
ESGGGLVQPGGSLRLSCAASGFTFTSSAMSWVRQAPGKGLEWVSRIDGSGGDTRYADSVKGRFTISRD
NSKNTLYLQMNSLRAEDTAVYYCAKGRRLWFGDPFDHWGQGTLVTVSS
F2-CDR-L1 (SEQ ID NO: 9)
KSSQSVLRSGNNKNRLA
F2-CDR-L2 (SEQ ID NO: 10)
DASTRES
F2-CDR-L3 (SEQ ID NO: 11)
QQQYNYPYT
F2-CDR-H1 (SEQ ID NO: 12)
GFTFTSSAMS
F2-CDR-H2 (SEQ ID NO: 13)
RIDGSGGDTRYADSVKG
F2-CDR-H3 (SEQ ID NO: 14)
GRRLWFGDPFDH
F3 (SEQ ID NO: 15)
DIVMTQSPDSLAVSLGERATINCKSSQSVLSSSNNKNRLAWYQQKPGQPPKLLIYAASTRESGVPDRFSG
SGSGTDFTLTISSLQAEDVAVYYCQQGYSSPWTFGQGTKVEIKGGGGSGGGGSGGGSGGGGSEVQLLE
SGGGLVQPGGSLRLSCAASGFTFSNYGMSWVRQAPGKGLEWVSRISGSGGYTNYADSVKGRFTISRDN
SKNTLYLQMNSLRAEDTAVYYCARVYFDGTTRYFDSWGPGTLVTVSS
F3-CDR-L1 (SEQ ID NO: 16)
KSSQSVLSSSNNKNRLA
F3-CDR-L2 (SEQ ID NO: 17)
AASTRES
F3-CDR-L3 (SEQ ID NO: 18)
QQGYSSPWT
F3-CDR-H1 (SEQ ID NO: 19)
GFTFSNYGMS
F3-CDR-H2 (SEQ ID NO: 20)
RISGSGGYTNYADSVKG
F3-CDR-H3 (SEQ ID NO: 21)
VYFDGTTRYFDS
F4 (SEQ ID NO: 22)
DIVMTQSPDSLAVSLGERATINCKSSQSVLRSGNNKNRLAWYQQKPGQPPKLLIYAASTRESGVPDRFSG
SGSGTDFTLTISSLQAEDVAVYYCQQSYRAPITFGQGTKVEIKGGGGSGGGGSGGGSGGGGSEVQLLES
GGGLVQPGGSLRLSCAASGFTFTDYAMSWVRQAPGKGLEWVSAIDGGGDSTYYADSVKGRFTISRDNS
KNTLYLQMNSLRAEDTAVYYCARAKFGSRRPERGMGYFDLWGRGTLVTVSS
F4-CDR-L1 (SEQ ID NO: 23)
KSSQSVLRSGNNKNRLA
F4-CDR-L2 (SEQ ID NO: 24)
AASTRES
F4-CDR-L3 (SEQ ID NO: 25)
QQSYRAPIT
F4-CDR-H1 (SEQ ID NO: 26)
GFTFTDYAMS
F4-CDR-H2 (SEQ ID NO: 27)
AIDGGGDSTYYADSVKG
F4-CDR-H3 (SEQ ID NO: 28)
AKFGSRRPERGMGYFDL
F5 (SEQ ID NO: 29)
DIVMTQSPDSLAVSLGERATINCKSSQSVLRSSNNKNRLAWYQQKPGQPPKLLIYAASTRESGVPDRFSG
SGSGTDFTLTISSLQAEDVAVYYCQQGYRAPFTFGQGTKVEIKGGGGSGGGGSGGGSGGGGSEVQLLE
SGGGLVQPGGSLRLSCAASGFTFSNYAMSWVRQAPGKGLEWVSRIDGSGGTTNYADSVKGRFTISRDN
SKNTLYLQMNSLRAEDTAVYYCAKSRGYNYGYFDYWGQGTLVTVSS
F5-CDR-L1 (SEQ ID NO: 30)
KSSQSVLRSSNNKNRLA
F5-CDR-L2 (SEQ ID NO: 31)
AASTRES
F5-CDR-L3 (SEQ ID NO: 32)
QQGYRAPFT
F5-CDR-H1 (SEQ ID NO: 33)
GFTFSNYAMS
F5-CDR-H2 (SEQ ID NO: 34)
RIDGSGGTTNYADSVKG
F5-CDR-H3 (SEQ ID NO: 35)
SRGYNYGYFDY
F6 (SEQ ID NO: 36)
DIVMTQSPDSLAVSLGERATINCKSSQSVLRSSNNKNRLAWYQQKPGQPPKLLIYAASTRESGVPDRFSG
SGSGTDFTLTISSLQAEDVAVYYCQQGYNAPLTFGQGTKVEIKGGGGSGGGGSGGGSGGGGSEVQLLE
SGGGLVQPGGSLRLSCAASGFTFTNYAMSWVRQAPGKGLEWVSRISGSGSSTSYADSVKGRFTISRDN
SKNTLYLQMNSLRAEDTAVYYCARFRLRSLYFDYWGQGTLVTVSS
F6-CDR-L1 (SEQ ID NO: 37)
KSSQSVLRSSNNKNRLA
F6-CDR-L2 (SEQ ID NO: 38)
AASTRES
F6-CDR-L3 (SEQ ID NO: 39)
QQGYNAPLT
F6-CDR-H1 (SEQ ID NO: 40)
GFTFTNYAMS
F6-CDR-H2 (SEQ ID NO: 41)
RISGSGSSTSYADSVKG
F6-CDR-H3 (SEQ ID NO: 42)
FRLRSLYFDY
F7 (SEQ ID NO: 43)
DIVMTQSPDSLAVSLGERATINCKSSQSVLRSSNNKNRLAWYQQKPGQPPKLLIYGASTRESGVPDRFSG
SGSGTDFTLTISSLQAEDVAVYYCQQGYNSPLTFGQGTKVEIKGGGGSGGGGSGGGSGGGGSEVQLLE
SGGGLVQPGGSLRLSCAASGFTFTNYAMSWVRQAPGKGLEWVSWISGRGGSTNYADSVKGRFTISRDN
SKNTLYLQMNSLRAEDTAVYYCARARARSSGYFDYWGQGTLVTVSS
F7-CDR-L1 (SEQ ID NO: 44)
KSSQSVLRSSNNKNRLA
F7-CDR-L2 (SEQ ID NO: 45)
GASTRES
F7-CDR-L3 (SEQ ID NO: 46)
QQGYNSPLT
F7-CDR-H1 (SEQ ID NO: 47)
GFTFTNYAMS
F7-CDR-H2 (SEQ ID NO: 48)
WISGRGGSTNYADSVKG
F7-CDR-H3 (SEQ ID NO: 49)
ARARSSGYFDY
F8 (SEQ ID NO: 50)
DIVMTQSPDSLAVSLGERATINCKSSQSVLRSGNNKNRLAWYQQKPGQPPKLLIYGASTRESGVPDRFS
GSGSGTDFTLTISSLQAEDVAVYYCQQGYSSPITFGQGTKVEIKGGGGSEVQLLESGGGLVQPGGSLRLS
CAASGFTFSDYYMSWVRQAPGKGLEWVSRIDGGGGSTYYADSVKGRFTISRDNSKNTLYLQMNSLRAE
DTAVYYCAKVRYPTGPYYFDYWGQGTLVTVSS
F8_18 AA linker (SEQ ID NO: 51)
DIVMTQSPDSLAVSLGERATINCKSSQSVLRSGNNKNRLAWYQQKPGQPPKLLIYGASTRESGVPDRFS
GSGSGTDFTLTISSLQAEDVAVYYCQQGYSSPITFGQGTKVEIKGGGGSGGGGSGGGSGGGGSEVQLL
ESGGGLVQPGGSLRLSCAASGFTFSDYYMSWVRQAPGKGLEWVSRIDGGGGSTYYADSVKGRFTISRD
NSKNTLYLQMNSLRAEDTAVYYCAKVRYPTGPYYFDYWGQGTLVTVSS
F8-CDR-L1 (SEQ ID NO: 52)
KSSQSVLRSGNNKNRLA
F8-CDR-L2 (SEQ ID NO: 53)
GASTRES
F8-CDR-L3 (SEQ ID NO: 54)
QQGYSSPIT
F8-CDR-H1 (SEQ ID NO: 55)
GFTFSDYYMS
F8-CDR-H2 (SEQ ID NO: 56)
RIDGGGGSTYYADSVKG
F8-CDR-H3 (SEQ ID NO: 57)
VRYPTGPYYFDY
F9 (SEQ ID NO: 58)
DIVMTQSPDSLAVSLGERATINCKSSQSVLRSNNNKNRLAWYQQKPGQPPKLLIYGASTRESGVPDRFS
GSGSGTDFTLTISSLQAEDVAVYYCQQGYSYPLTFGQGTKVEIKGGGGSGGGGSGGGSGGGGSEVQLF
ESGGGLVQPGGSLRLSCAASGFTFTDYAMSWVRQAPGKGLEWVSAIQGGGGGTRYADSVKGRFTISRD
NSKNTLYLQMNSLRAEDTAVYYCARDRRPTVTTSYFDYWGQGTLVTVSS
F9-CDR-L1 (SEQ ID NO: 59)
KSSQSVLRSNNNKNRLA
F9-CDR-L2 (SEQ ID NO: 60)
GASTRES
F9-CDR-L3 (SEQ ID NO: 61)
QQGYSYPLT
F9-CDR-H1 (SEQ ID NO: 62)
GFTFTDYAMS
F9-CDR-H2 (SEQ ID NO: 63)
AIQGGGGGTRYADSVKG
F9-CDR-H3 (SEQ ID NO: 64)
DRRPTVTTSYFDY
F10 (SEQ ID NO: 65)
DIVMTQSPDSLAVSLGERATINCKSSQSVLRSGNNKNRLAWYQQKPGQPPKLLIYWASTRESGVPDRFS
GSGSGTDFTLTISSLQAEDVAVYYCQQSYNRPLTFGQGTKVEIKGGGGSGGGGSGGGSGGGGSEVQLL
ESGGGLVQPGGSLRLSCAASGFTFSSYQMSWVRQAPGKGLEWVSEISGYGGSTKYADSVKGRFTISRD
NSKNTLYLQMNSLRAEDTAVYYCARRNAAFDIWGQGTMVTVSS
F10-CDR-L1 (SEQ ID NO: 66)
KSSQSVLRSGNNKNRLA
F10-CDR-L2 (SEQ ID NO: 67)
WASTRES
F10-CDR-L3 (SEQ ID NO: 68)
QQSYNRPLT
F10-CDR-H1 (SEQ ID NO: 69)
GFTFSSYQMS
F10-CDR-H2 (SEQ ID NO: 70)
EISGYGGSTKYADSVKG
F10-CDR-H3 (SEQ ID NO: 71)
RNAAFDI
F11 (SEQ ID NO: 72)
DIVMTQSPDSLAVSLGERATINCKSSQSVLRSDNNENRLAWYQQKPGQPPKLLIYAASTRESGVPDRFSG
SGSGTDFTLTISSLQAEDVAVYYCQQYYREPITFGQGTKVEIKGGGGSGGGGSGGGSGGGGSEVQLLES
GGGLVQPGGSLRLSCAASGFTFTSYGMSWVRQAPGKGLEWVSGISGRGGSTSYADSVKGRFTISRDNS
KNTLYLQMNSLRAEDTAVYYCARTRGRIAARRGWFDPWGQGTLVTVSS
F11-CDR-L1 (SEQ ID NO: 73)
KSSQSVLRSDNNENRLA
F11-CDR-L2 (SEQ ID NO: 74)
AASTRES
F11-CDR-L3 (SEQ ID NO: 75)
QQYYREPIT
F11-CDR-H1 (SEQ ID NO: 76)
GFTFTSYGMS
F11-CDR-H2 (SEQ ID NO: 77)
GISGRGGSTSYADSVKG
F11-CDR-H3 (SEQ ID NO: 78)
TRGRIAARRGWFDP
F12 (SEQ ID NO: 79)
DIVMTQSPDSLAVSLGERATINCKSSQSVLRSDNNKNRLAWYQQKPGQPPKLLIYGASTRESGVPDRFS
GSGSGTDFTLTISSLQAEDVAVYYCQQGYSSPWTFGQGTKVEIKGGGGSGGGGSGGGSGGGGSEVQLL
ESGGGLVQPGGSLRLSCAASGFTFTSTAMSWVRQAPGKGLEWVSAISGRGGSTKYADSVKGRFTISRD
NSKNTLYLQMNSLRAEDTAVYYCARASKKSPIAAAGTFDLWGQGTLVTVSS
F12-CDR-L1 (SEQ ID NO: 80)
KSSQSVLRSDNNKNRLA
F12-CDR-L2 (SEQ ID NO: 81)
GASTRES
F12-CDR-L3 (SEQ ID NO: 82)
QQGYSSPWT
F12-CDR-H1 (SEQ ID NO: 83)
GFTFTSTAMS
F12-CDR-H2 (SEQ ID NO: 84)
AISGRGGSTKYADSVKG
F12-CDR-H3 (SEQ ID NO: 85)
ASKKSPIAAAGTFDL
F13 (SEQ ID NO: 86)
DIVMTQSPDSLAVSLGERATINCKSSQSVLRSGNNKNRLAWYQQKPGQPPKLLIYGASTRESGVPDRFS
GSGSGTDFTLTISSLQAEDVAVYYCQQYYNRPYTFGQGTKVEIKGGGGSGGGGSGGGSGGGGSEVQLL
ESGGGLVQPGGSLRLSCAASGFTFTDYGMSWVRQAPGKGLEWVSGISGSGGSTYYADSVKGRFTISRD
NSKNTLYLQMNSLRAEDTAVYYCAKSRRRDYNWFDPWGQGTLVTVSS
F13-CDR-L1 (SEQ ID NO: 87)
KSSQSVLRSGNNKNRLA
F13-CDR-L2 (SEQ ID NO: 88)
GASTRES
F13-CDR-L3 (SEQ ID NO: 89)
QQYYNRPYT
F13-CDR-H1 (SEQ ID NO: 90)
GFTFTDYGMS
F13-CDR-H2 (SEQ ID NO: 91)
GISGSGGSTYYADSVKG
F13-CDR-H3 (SEQ ID NO: 92)
SRRRDYNWFDP
F14 (SEQ ID NO: 93)
DIVMTQSPDSLAVSLGERATINCKSSQSVLRSGNNKNRLAWYQQKPGQPPKLLIYAASTRESGVPDRFSG
SGSGTDFTLTISSLQAEDVAVYYCQQGYREPLTFGQGTKVEIKGGGGSGGGGSGGGSGGGGSEVQLLE
SGGGLVQPGGSLRLSCAASGFTFSNYYMSWVRQAPGKGLEWVSAISGSGGQTSYADSVKGRFTISRDN
SKNTLYLQMNSLRAEDTAVYYCARHRRGRGAMGYFDYWGQGTLVTVSS
F14-CDR-L1 (SEQ ID NO: 94)
KSSQSVLRSGNNKNRLA
F14-CDR-L2 (SEQ ID NO: 95)
AASTRES
F14-CDR-L3 (SEQ ID NO: 96)
QQGYREPLT
F14-CDR-H1 (SEQ ID NO: 97)
GFTFSNYYMS
F14-CDR-H2 (SEQ ID NO: 98)
AISGSGGQTSYADSVKG
F14-CDR-H3 (SEQ ID NO: 99)
HRRGRGAMGYFDY
F15 (SEQ ID NO: 100)
DIVMTQSPDSLAVSLGERATINCKSSQSVLRSGNNKNRLAWYQQKPGQPPKLLIYAASTRESGVPDRFSG
SGSGTDFTLTISSLQAEDVAVYYCQQGYSYPYTFGQGTKVEIKGGGGSGGGGSGGGSGGGGSEVQLLE
SGGGLVQPGGSLRLSCAASGFTFSSYGMSWVRQAPGKGLEWVSDIDGSGGSTYYADSVKGRFTISRDN
SKNTLYLQMNSLRAEDTAVYYCARDRRARSHYFDYWGQGTLVTVSS
F15-CDR-L1 (SEQ ID NO: 101)
KSSQSVLRSGNNKNRLA
F15-CDR-L2 (SEQ ID NO: 102)
AASTRES
F15-CDR-L3 (SEQ ID NO: 103)
QQGYSYPYT
F15-CDR-H1 (SEQ ID NO: 104)
GFTFSSYGMS
F15-CDR-H2 (SEQ ID NO: 105)
DIDGSGGSTYYADSVKG
F15-CDR-H3 (SEQ ID NO: 106)
DRRARSHYFDY
F16 (SEQ ID NO: 107)
DIVMTQSPDSLAVSLGERATINCKSSQSVLSSYNNKNRLAWYQQKPGQPPKLLIYAASTRESGVPDRFSG
SGSGTDFTLTISSLQAEDVAVYYCQQGYSRPITFGQGTKVEIKGGGGSGGGGSGGGSGGGGSEVQLLES
GGGLVQPGGSLRLSCAASGFTFTSYQMSWVRQAPGKGLEWVSVIDGSGGYTRYADSVKGRFTISRDNS
KNTLYLQMNSLRAEDTAVYYCASPRRGKGSYFDYWGQGTLVTVSS
F16-CDR-L1 (SEQ ID NO: 108)
KSSQSVLSSYNNKNRLA
F16-CDR-L2 (SEQ ID NO: 109)
AASTRES
F16-CDR-L3 (SEQ ID NO: 110)
QQGYSRPIT
F16-CDR-H1 (SEQ ID NO: 111)
GFTFTSYQMS
F16-CDR-H2 (SEQ ID NO: 112)
VIDGSGGYTRYADSVKG
F16-CDR-H3 (SEQ ID NO: 113)
PRRGKGSYFDY
F17 (SEQ ID NO: 114)
DIVMTQSPDSLAVSLGERATINCKSSQSVLRSGNNKNRLAWYQQKPGQPPKLLIYGASTRESGVPDRFS
GSGSGTDFTLTISSLQAEDVAVYYCQQGYSAPITFGQGTKVEIKGGGGSGGGGSGGGSGGGGSEVQLL
ESGGGLVQPGGSLRLSCAASGFTFSSYAMSWVRQAPGKGLEWVSAISGSGGSTRYADSVKGRFTISRD
NSKNTLYLQMNSLRAEDTAVYYCARARSSSGGYFDYWGQGTLVTVSS
F17-CDR-L1 (SEQ ID NO: 115)
KSSQSVLRSGNNKNRLA
F17-CDR-L2 (SEQ ID NO: 116)
GASTRES
F17-CDR-L3 (SEQ ID NO: 117)
QQGYSAPIT
F17-CDR-H1 (SEQ ID NO: 118)
GFTFSSYAMS
F17-CDR-H2 (SEQ ID NO: 119)
AISGSGGSTRYADSVKG
F17-CDR-H3 (SEQ ID NO: 120)
ARSSSGGYFDY
C9-VLVH (SEQ ID NO: 121)
QPVLTQPPSASGTPGQRVTISCSGSSSNIGNNPVNWYQQLPGTAPKLLIYRNNQRPSGVPDRFSGSKSG
TSASLAISGLRSEDEAHYYCAAWNDRLSGPRVFGGGTKLTVLGGGGSGGGGSGGGGSGGGQVQLVQS
GGGLAQPGGSLRLSCAASGFTFRSYWMTWARQAPGKGLEWVATIKQDGSEKYYVDSVKGRFTISRDDA
TNSLFLQMNSLRAEDTALYYCARARNMDVWGQGTTVTVSS
C9-CDR-L1 (SEQ ID NO: 122)
SGSSSNIGNNPVN
C9-CDR-L2 (SEQ ID NO: 123)
RNNQRPS
C9-CDR-L3 (SEQ ID NO: 124)
AAWNDRLSGPRV
C9-CDR-H1 (SEQ ID NO: 125)
GFTFRSYWMT
C9-CDR-H2 (SEQ ID NO: 126)
TIKQDGSEKYYVDSVKG
C9-CDR-H3 (SEQ ID NO: 127)
ARNMDV
B3-VLVH (SEQ ID NO: 128)
DIVMTQSPSSMSASVGDRVTITCRASQGLSNRLAWYQQKPGKAPKLLIYAASSLQSGVPSRFSGSGSGT
DFTLTISRLQPEDFATYYCQQANSFPLTFGQGTRLEIKGGGGSGGGGSGGGSGGGGSQVQLVQSGAEV
KKPGASVKVSCKASGYTFTGYYMHWVRQAPGQGLEWMGWINPNSGGTNYAQKFQGRVTMTRDTSIST
AYMELSRLRSDDTAVYYCARGGSSGWRKYFQHWGQGTLVTVSS
B3-CDR-L1 (SEQ ID NO: 129)
RASQGLSNRLA
B3-CDR-L2 (SEQ ID NO: 130)
AASSLQS
B3-CDR-L3 (SEQ ID NO: 131)
QQANSFPLT
B3-CDR-H1 (SEQ ID NO: 132)
GYTFTGYYMH
B3-CDR-H2 (SEQ ID NO: 133)
WINPNSGGTNYAQKFQG
B3-CDR-H3 (SEQ ID NO: 134)
GGSSGWRKYFQH
VAR2CSA (SEQ ID NO: 135)
MDSTSTIANKIEEYLGAKSDDSKIDELLKADPSEVEYYRSGGDGDYLKNNICKITVNHSDSGKYDPCEKKL
PPYDDNDQWKCQQNSSDGSGKPENICVPPRRERLCTYNLENLKFDKIRDNNAFLADVLLTARNEGEKIVQ
NHPDTNSSNVCNALERSFADLADIIRGTDQWKGTNSNLEKNLKQMFAKIRENDKVLQDKYPKDQKYTKL
REAWWNANRQKVWEVITCGARSNDLLIKRGWRTSGKSDRKKNFELCRKCGHYEKEVPTKLDYVPQFLR
WLTEWIEDFYREKQNLIDDMERHREECTREDHKSKEGTSYCSTCKDKCKKYCECVKKWKTEWENQENK
YKDLYEQNKNKTSQKNTSRYDDYVKDFFEKLEANYSSLENYIKGDPYFAEYATKLSFILNPSDANNPSGET
ANHNDEACNCNESGISSVGQAQTSGPSSNKTCITHSSIKTNKKKECKDVKLGVRENDKDLKICVIEDTSL
SGVDNCCCQDLLGILQENCSDNKRGSSSNDSCDNKNQDECQKKLEKVFASLTNGYKCDKCKSGTSRSK
KKWIWKKSSGNEEGLQEEYANTIGLPPRTQSLYLGNLPKLENVCEDVKDINFDTKEKFLAGCLIVSFHEGK
NLKKRYPQNKNSGNKENLCKALEYSFADYGDLIKGTSIWDNEYTKDLELNLQNNFGKLFGKYIKKNNTAE
QDTSYSSLDELRESWWNTNKKYIWTAMKHGAEMNITTCNADGSVTGSGSSCDDIPTIDLIPQYLRFLQE
WVENFCEQRQAKVKDVITNCKSCKESGNKCKTECKTKCKDECEKYKKFIEACGTAGGGIGTAGSPWSKR
WDQIYKRYSKHIEDAKRNRKAGTKNCGTSSTTNAAASTDENKCVQSDIDSFFKHLIDIGLTTPSSYLSNV
LDDNICGADKAPWTTYTTYTTTEKCNKERDKSKSQSSDTLVVVNVPSPLGNTPYRYKYACQCKIPTNEET
CDDRKEYMNQWSCGSARTMKRGYKNDNYELCKYNGVDVKPTTVRSNSSKLDGNDVTFFNLFEQWNKEI
QYQIEQYMTNANISCIDEKEVLDSVSDEGTPKVRGGYEDGRNNNTDQGTNCKEKCKCYKLWIEKINDQ
WGKQKDNYNKFRSKQIYDANKGSQNKKVVSLSNFLFFSCWEEYIQKYFNGDWSKIKNIGSDTFEFLIKK
CGNNSAHGEEIFNEKLKNAEKKCKENESTDTNINKSETSCDLNATNYIRGCQSKTYDGKIFPGKGGEKQ
WICKDTIIHGDTNGACIPPRTQNLCVGELWDKSYGGRSNIKNDTKELLKEKIKNAIHKETELLYEYHDTGT
AIISKNDKKGQKGKNDPNGLPKGFCHAVQRSFIDYKNMILGTSVNIYEHIGKLQEDIKKIIEKGTPQQKDK
IGGVGSSTENVNAWWKGIEREMWDAVRCAITKINKKNNNSIFNGDECGVSPPTGNDEDQSVSWFKEW
GEQFCIERLRYEQNIREACTINGKNEKKCINSKSGQGDKIQGACKRKCEKYKKYISEKKQEWDKQKTKYE
NKYVGKSASDLLKENYPECISANFDFIFNDNIEYKTYYPYGDYSSICSCEQVKYYKYNNAEKKNNKSLCYE
KDNDMTWSKKYIKKLENGRSLEGVYVPPRRQQLCLYELFPIIIKNEEGMEKAKEELLETLQIVAEREAYYLW
KQYNPTGKGIDDANKKACCAIRGSFYDLEDIIKGNDLVHDEYTKYIDSKLNEIFGSSDTNDIDTKRARTD
WWENETITNGTDRKTIRQLVWDAMQSGVRYAVEEKNENFPLCMGVEHIGIAKPQFIRWLEEWTNEFCEK
YTKYFEDMKSKCDPPKRADTCGDNSNIECKKACANYTNWLNPKRIEWNGMSNYYNKIYRKSNKESEGG
KDYSMIMAPTVIDYLNKRCHGEINGNYICCSCKNIGAYNTTSGTVNKKLQKKETECEEEKGPLDLMNEVL
NKMDKKYSAHKMKCTEVYLEHVEEQLNEIDNAIKDYKLYPLDRCFDDQTKMKVCDLIADAIGCKDKTKLD
ELDEWNDMDLRGTYNKHKGVLIPPRRRQLCFSRIVRGPANLRSLNEFKEEILKGAQSEGKFLGNYYKEHK
DKEKALEAMKNSFYDYEDIIKGTDMLTNIEFKDIKIKLDRLLEKETNNTKKAEDWWKTNKKSIWNAMLCG
YKKSGNKIIDPSWCTIPTTETPPQFLRWIKEWGTNVCIQKQEHKEYVKSKCSNVTNLGAQASESNNCTSE
IKKYQEWSRKRSIRWETISKRYKKYKRMDILKDVKEPDANTYLREHCSKCPCGFNDMEEMNNNEDNEKE
AFKQIKEQVKIPAELEDVIYRIKHHEYDKGNDYICNKYKNIHDRMKKNNGNFVTDNFVKKSWEISNGVLIP
PRRKNLFLYIDPSKICEYKKDPKLFKDFIYWSAFTEVERLKKAYGGARAKVVHAMKYSFTDIGSIIKGDDM
MEKNSSDKIGKILGDTDGQNEKRKKWWDMNKYHIWESMLCGYREAEGDTETNENCRFPDIESVPQFLR
WFQEWSENFCDRRQKLYDKLNSECISAECTNGSVDNSKCTHACVNYKNYILTKKTEYEIQTNKYDNEFK
NKNSNDKDAPDYLKEKCNDNKCECLNKHIDDKNKTWKNPYETLEDTFKSKCDCPKPLPSPIKPDDLPPQA
DEPFDPTILQTTIPFGIALALGSIAFLFMKVIYIYIYVCCICMYVCMYVCMYVCM
VAR2CSA-ID1-ID2a (SEQ ID NO: 136)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLYLGNLPK
LENVCEDVKDINFDTKEKFLAGCLIVSFHEGKNLKKRYPQNKNSGNKENLCKALEYSFADYGDLIKGTSIW
DNEYTKDLELNLQNNFGKLFGKYIKKNNTAEQDTSYSSLDELRESWWNTNKKYIWTAMKHGAEMNITTC
NADGSVTGSGSSCDDIPTIDLIPQYLRFLQEWVENFCEQRQAKVKDVITNCKSCKESGNKCKTECKTKCK
DECEKYKKFIEACGTAGGGIGTAGSPWSKRWDQIYKRYSKHIEDAKRNRKAGTKNCGTSSTTNAAASTD
ENKCVQSDIDSFFKHLIDIGLTTPSSYLSNVLDDNICGADKAPWTTYTTYTTTEKCNKERDKSKSQSSDTL
VVVNVPSPLGNTPYRYKYACQCKIPTNEETCDDRKEYMNQWSCGSARTMKRGYKNDNYELCKYNGVDV
KPTTVRSNSSKLD
GS-Linker-1 (SEQ ID NO: 137)
GGGGSGGGGSGGGSGGGGS
GS-Linker-2 (SEQ ID NO: 138)
GGGGSGGGGSGGGGSGGG
GS-Linker-3 (SEQ ID NO: 139)
GGGSGGGS

Generally, when referring to an antibody or antibody variant of the present invention by name (i.e. F1-F17, C9 and B3), the general, unspecific reference (such as “C9” or “C9 antibody”) to such an antibody name is intended to relate generally to any antibody format or antibody variant, which comprises the 6 corresponding CDR sequences which are set forth above. So unless more specific details as to the specific construct format is indicated, then:
“an F1 antibody” or “F1” refers not only to SEQ ID NO: 1 but also to an antibody or antibody variant format described herein, which comprises the 6 CDR sequences set forth in SEQ ID Nos: 2-7;
“an F2 antibody” or “F2” refers not only to SEQ ID NO: 8 but also to an antibody or antibody variant format described herein, which comprises the 6 CDR sequences set forth in SEQ ID Nos: 9-14;
“an F3 antibody” or “F3” refers not only to SEQ ID NO: 15 but also to an antibody or antibody variant format described herein, which comprises the 6 CDR sequences set forth in SEQ ID Nos: 16-21;
“an F4 antibody” or “F4” refers not only to SEQ ID NO: 22 but also to an antibody or antibody variant format described herein, which comprises the 6 CDR sequences set forth in SEQ ID Nos: 23-28;
“an F5 antibody” or “F5” refers not only to a polypeptide consisting of SEQ ID NO: 29 but also to an antibody or antibody variant format described herein, which comprises the 6 CDR sequences set forth in SEQ ID Nos: 30-35;
“an F6 antibody” or “F6” refers not only to a polypeptide consisting of SEQ ID NO: 36 but also to an antibody or antibody variant format described herein, which comprises the 6 CDR sequences set forth in SEQ ID Nos: 37-42;
“an F7 antibody” or “F7” refers not only to a polypeptide consisting of SEQ ID NO: 43 but also to an antibody or antibody variant format described herein, which comprises the 6 CDR sequences set forth in SEQ ID Nos: 44-49;
“an F8 antibody” or “E8” refers not only to a polypeptide consisting of SEQ ID NO: 50 but also to an antibody or antibody variant format described herein, which comprises the 6 CDR sequences set forth in SEQ ID Nos: 52-57;
“an F9 antibody” or “F9” refers not only to a polypeptide consisting of SEQ ID NO: 58 but also to an antibody or antibody variant format described herein, which comprises the 6 CDR sequences set forth in SEQ ID Nos: 59-64;
“an F10 antibody” or “F10” refers not only to a polypeptide consisting of SEQ ID NO: 65 but also to an antibody or antibody variant format described herein, which comprises the 6 CDR sequences set forth in SEQ ID Nos: 66-71;
“an F11 antibody” or “F11” refers not only to a polypeptide consisting of SEQ ID NO: 72 but also to an antibody or antibody variant format described herein, which comprises the 6 CDR sequences set forth in SEQ ID Nos: 73-78;
“an F12 antibody” or “E12” refers not only to a polypeptide consisting of SEQ ID NO: 79 but also to an antibody or antibody variant format described herein, which comprises the 6 CDR sequences set forth in SEQ ID Nos: 80-85;
“an F13 antibody” or “E13” refers not only to a polypeptide consisting of SEQ ID NO: 86 but also to an antibody or antibody variant format described herein, which comprises the 6 CDR sequences set forth in SEQ ID Nos: 87-92;
“an F14 antibody” or “E14” refers not only to a polypeptide consisting of SEQ ID NO: 83 but also to an antibody or antibody variant format described herein, which comprises the 6 CDR sequences set forth in SEQ ID Nos: 84-99;
“an F15 antibody” or “F15” refers not only to a polypeptide consisting of SEQ ID NO: 100 but also to an antibody or antibody variant format described herein, which comprises the 6 CDR sequences set forth in SEQ ID Nos: 101-106;
“an F16 antibody” or “E16” refers not only to a polypeptide consisting of SEQ ID NO: 107 but also to an antibody or antibody variant format described herein, which comprises the 6 CDR sequences set forth in SEQ ID Nos: 108-113;
“an F17 antibody” or “F17” refers not only to a polypeptide consisting of SEQ ID NO: 114 but also to an antibody or antibody variant format described herein, which comprises the 6 CDR sequences set forth in SEQ ID Nos: 115-120;
“a C9 antibody” or “C9” refers not only to a polypeptide consisting of SEQ ID NO: 121 but also to an antibody or antibody variant format discussed herein, which comprises the 6 CDR sequences set forth in SEQ ID Nos: 122-127; and
“a B3 antibody” or “B3” refers not only to a polypeptide consisting of SEQ ID NO: 128 but also to an antibody or antibody variant format described herein, which comprises the 6 CDR sequences set forth in SEQ ID Nos: 129-134.

### Example 1

Identification of ofCS-Specific Antibodies

To make a high affinity IgG monoclonal antibody towards ofCS the obvious starting point was to immunize mice with purified ofCS using immunization strategies that could break murine self-tolerance, followed by ex vivo hybridoma or EBV transformation technologies to make monoclonal antibodies. However multiple attempts using mouse immunization against biotinylated placental CS or recombinant ofCS-carrying proteoglycans (on a decorin or serglycin backbone), failed to identify any high affinity and highly specific ofCS-specific antibodies. The testing of the mice sera, obtained after series of immunizations, using both antigen ELISA and flow cytometry on ofCS positive cancer cells did not identify any clones with significant binding over controls. Further screening for monoclonals using FACS sorting also did not give any specific clone. Multiple iterations with changing strategy of conjugation of ofCS to keyhole limpet hemocyanin, a highly immunogenic T-cell dependent antigen along with changing adjuvant strains did not give ofCS-specific monoclonal antibodies. Further we attempted conjugation to virus-like particles but without obtaining significantly positive immune sera. Immunizations were repeated in rats and chickens to make an ofCS-specific IgY without success.

A breakthrough was only achieved when the strategy was changed to using phage display. Key to the success of generating ofCS-specific antibodies using phage display is a combination of having the exact ofCS epitope available for panning and a panning/selection strategy using the proven rVAR2 specificity for ofCS. The key reagents in the panning procedure were highly purified and well-characterized ofCS as well as recombinant ofCSPGs homogenously presenting an ofCS modification on a decorin backbone. For this purpose, the decorin gene was cloned into a pCDNA 3.1 vector under a CMV promoter with a 6×His tag and produced in Chinese Hamster Ovary cells in secreted form. The protein was purified using IMAC chromatography and desalted into PBS. SDS PAGE and binding to VAR2CSA protein with or without chondroitinase ABC (ChABC) treatment was used to validate the protein. The binding of VAR2CSA to recombinant decorin was relatively lower than to native decorin; the reason of which was identified to be low levels of ofCS using both SDS PAGE and alcian blue staining. This was likely an artefact from recombinant expression where the ofCS enzymatic machinery cannot follow the gene expression of the protein back bone. The challenge with less abundance of CS on the recombinantly produced protein was solved by enrichment of CS. CS-enriched decorin was obtained using anion exchange chromatography by exploiting the high negative charge of CS. OfCS from placental tissue was purified and quality controlled as described in Beaudet et al., Glycoconj. J., 2014. Biotinylated ofCS or recombinant ofCSPG was used for biopanning of phage display libraries being constructed from either fully naïve Homo sapiens immunoglobulin repertoire, fully synthetic Fab or semi-synthetic ScFv with a combined variation of more than 10¹² clones. Negative selection was done in an iterative process using chondroitinase ABC treatment of the reagents before negative selection and/or on purified heparan sulfate proteoglycan (HSPG) as a source of very charged non-ofCS glycan. Elution of bound phages was done either by pH elution or by competition with rVAR2, competing for the ofCS epitope. After 3^{rd} and 4^{th} rounds of biopanning, selected clones were tested for binding to ofCS in ELISA. And specificity was validated by testing binding to HSPG, various sources of ofCS and by competition ELISA with rVAR2. Deep sequencing of selected phages was performed to ensure all clones were identified. In total, from six different phage display libraries we obtained 19 antibody fragments with specificity to ofCS when presented in phage context.

### Example 2

### Production of Antibodies and Antibody Fragments

scFv expression and purification: The amino acid sequence of the phage display-derived ofCS binding VL and VH domains were produced as single-chain variable fragments (scFv) using E. coli cells. The VL and VH domains were genetically fused using an 18 amino acid linker. A V5-tag, 6×HIS-tag and SpyTag were added to the C-terminus of the scFv. The scFv was expressed in E. coli Shuffle cells (NEB) and purified from the soluble fraction after cell lysis. A 2-step purification was performed using a HisTrap HP (Cytiva) column for capture, followed by cation exchange chromatography using a HiTrap SP HP column (Cytiva) for polishing.

Dimeric SpyCatcher expression and purification: The SpyCatcher molecule was cloned and expressed as a dimeric construct. A short flexible linker GGGSGGGS (SEQ ID NO: 139) was used to tether two SpyCatcher molecules together. Also, a 6×HIS-tag was added to the C-terminus. The construct was expressed in E. coli BL21 cells and was purified from the soluble fraction after cell lysis. A 2-step purification was performed using a HisTrap HP (Cytiva) column for capture, followed by anion exchange chromatography using a HiTrap Q HP column (Cytiva) for polishing.

IgG expression and purification: Full human IgG antibodies containing the ofCS VL and VH domains for binding were produced in CHO cells. The target VH and VL domains were ordered as individual genes and subcloned into a pTRIOZ (Invivogen) vector containing individual cassettes for heavy and light chain of human IgG1 kappa expression. Protein expression was achieved using the expiCHO transient transfection system (Thermo Fisher) following the product guidelines. The media was harvested after 7-9 days transfection and the produced IgG was captured using a Protein A column. The protein was further purified using an anion exchange column in flow-through mode, yielding the monomeric mAb.

The anti-ofCS scFvs produced at high yields (>20 mg/L) and appeared as clean monomeric proteins on SDS-page. To facilitate dimerization, we used a split-protein system based on the spytag sequence which forms an isopeptide bond with a corresponding spycatchertag. The spytagged ofCS scFvs were conjugated in a molar ratio of 2:1 to dimeric Spycatcher, thereby creating a bi-valent construct similar to the variable regions of an IgG. All constructs remained soluble and retained ligand binding. Increasing valency going from monomeric to dimeric increased binding.

### Example 3

Specificity of ofCS Antibodies and Antibody Fragments

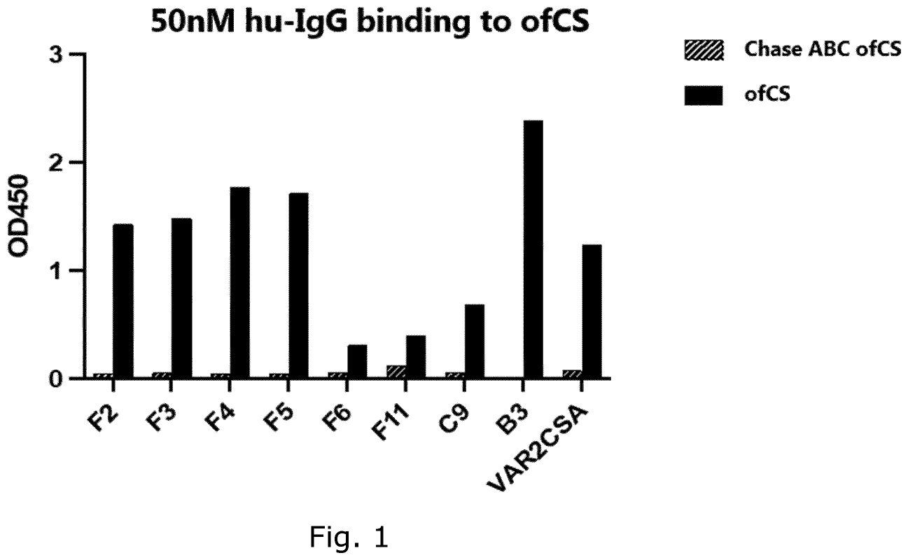

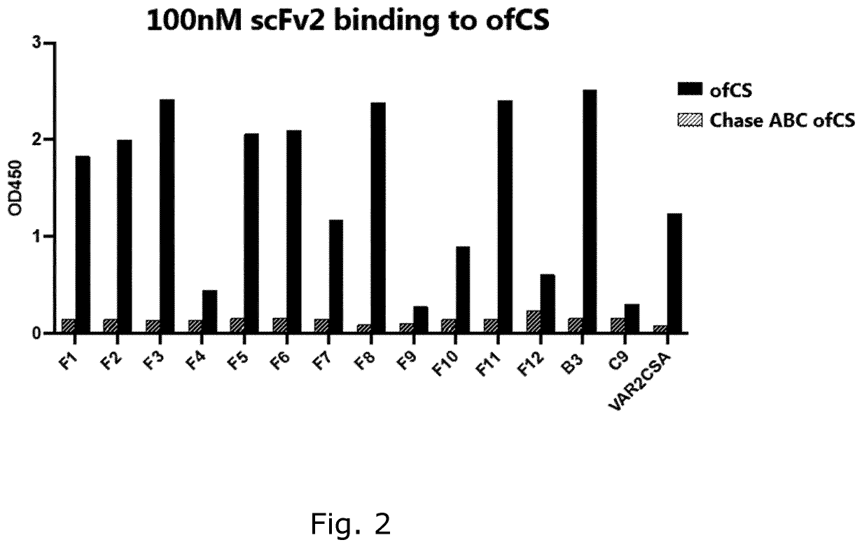

Specificity of ofCS binding could likely change going from a format where the sequence was presented at high density on phage to a therapeutic relevant molecule like an scFv2, Fab or IgG. To address this, we tested both scFv dimers and IgGs for binding to ofCS, by ELISA, as described (Salanti and Clausen et al., Cancer Cell, 2015). To determine if the interaction indeed was between the antibody reagent and the glycosaminoglycan chain, we also tested binding after chondroitinase treatment of ofCS. FIG. 1 shows binding of selected IgGs and FIG. 2 shows binding of selected ScFvs. As a control we included rVAR2 (VAR2CSA). For all tested antibodies and antibody fragments we saw binding to ofCS, some even to a higher extent than VAR2CSA. The binding was fully abrogated after chondroitinase treatment of the target reagent, which shows that there was full specificity towards a chondroitin type glycosaminoglycan (FIGS. 1 and 2). Inhibition using soluble chondroitin sulfates demonstrated mixed and overlapping specificities towards CSA, CSC and CSB (dermatan sulfate) (data not shown), likely demonstrating the previously described hybrid nature of ofCS.

We then tested binding of the antibodies or antibody fragments to recombinant ofCSPG (ofCS on a decorin backbone) (Tables 1 and 3) and commercial HSPG from Merck (Sigma-Aldrich; H4777) (Tables 2 and 4). The tables demonstrate a very clear preference for CSPG binding with limited binding to HSPG. Altogether the data demonstrate that the produced ofCS biopanned antibody fragments and antibodies bind specifically to chondroitin sulfates in a similar way as the recombinant malaria VAR2 protein and with limited binding to the charged HSPG molecule.

We demonstrated that VAR2CSA and the ofCS binding antibodies share a common epitope in a competition ELISA setup. In brief, an ELISA plate was coated with ofCS and binding of an ofCS antibody was measured in the presence or absence of rVAR2. This was enabled by having a non-V5 tagged VAR2 and V5-tagged anti-ofCS scFvs. As an example, the OD-value of F2 anti-ofCS to ofCS at a 400 nM concentration was measured to 2.0 and addition of 3 μM VAR2 protein decreased binding to 1.2 OD. Similarly, the OD-value of C9 binding to ofCSPG at 25 nM was 1.4, and in competition with 3 μM VAR2 the OD was reduced to 0.2.

TABLE 1
ofCS IgG binding to CSPG

| | | | | Antibody |
|---|---|---|---|---|
| | Concentration | CSPG | Detection | control |
| Protein | (nM) | (OD450) | antibody | (OD450) |
| C9 hu-IgG | 20 | 1.4717 | Anti hu-IgG HRP | 0.0918 |
| B3 hu-IgG | 25 | 2.3757 | Anti hu-IgG HRP | 0.0001 |
| F2 hu-IgG | 25 | 2.2316 | Anti hu-IgG HRP | 0.2319 |
| F5 hu-IgG | 25 | 1.1806 | Anti hu-IgG HRP | 0.2319 |
| F6 hu-IgG | 25 | 1.6644 | Anti hu-IgG HRP | 0.2319 |
| F11 hu-IgG | 25 | 1.963 | Anti hu-IgG HRP | 0.2319 |
| VAR2CSA | 25 | 1.7697 | Anti V5 HRP | 0.1136 |

TABLE 2
ofCS IgG binding to HSPG

| | | | | Antibody |
|---|---|---|---|---|
| | Concentration | HSPG | Detection | control |
| Protein | (nM) | (OD450) | antibody | (OD450) |
| C9 hu-IgG | 25 | 0.0667 | Anti hu-IgG HRP | 0.007 |
| B3 hu-IgG | 25 | 0.1102 | Anti hu-IgG HRP | 0.0467 |
| VAR2CSA | 25 | 0.3315 | Anti V5 HRP | 0.045 |

TABLE 3
ofCS scFv binding to CSPG

| | | | | Antibody |
|---|---|---|---|---|
| | Concentration | CSPG | Detection | control |
| Protein | (nM) | (OD450) | antibody | (OD450) |
| F1 scFv dimer | 100 | 0.587 | Anti V5 HRP | 0.1137 |
| F2 scFv dimer | 100 | 1.0498 | Anti V5 HRP | 0.1137 |
| F4 scFv dimer | 100 | 0.2115 | Anti V5 HRP | 0.1137 |
| F5 scFv dimer | 100 | 1.6654 | Anti V5 HRP | 0.1137 |
| F6 scFv dimer | 100 | 1.1957 | Anti V5 HRP | 0.1137 |
| F7 scFv dimer | 100 | 0.1063 | Anti V5 HRP | 0.1137 |
| F8 scFv dimer | 100 | 0.1025 | Anti V5 HRP | 0.0843 |
| F9 scFv dimer | 100 | 0.1998 | Anti V5 HRP | 0.0684 |
| F10 scFv dimer | 100 | 0.1159 | Anti V5 HRP | 0.0684 |
| F11 scFv dimer | 100 | 2.7901 | Anti V5 HRP | 0.0684 |
| F12 scFv dimer | 100 | 0.1241 | Anti V5 HRP | 0.0684 |
| B3 scFv dimer | 100 | 2.0977 | Anti V5 HRP | 0.1137 |
| C9 scFv dimer | 100 | 1.0249 | Anti V5 HRP | 0.1137 |
| VAR2CSA | 100 | 2.3814 | Anti V5 HRP | 0.1137 |

TABLE 4
ofCS scFv binding to HSPG

| | | | | Antibody |
|---|---|---|---|---|
| | Concentration | HSPG | Detection | control |
| Protein | (nM) | (OD450) | antibody | (OD450) |
| F3 scFv dimer | 100 | 0.0929 | Anti V5 HRP | 0.1110 |
| F4 scFv dimer | 100 | 0.1445 | Anti V5 HRP | 0.1110 |
| F5 scFv dimer | 100 | 0.1446 | Anti V5 HRP | 0.1110 |
| F6 scFv dimer | 100 | 0.1664 | Anti V5 HRP | 0.1110 |
| F7 scFv dimer | 100 | 0.1013 | Anti V5 HRP | 0.1110 |
| F8 scFv dimer | 100 | 0.1004 | Anti V5 HRP | 0.097 |
| F9 scFv dimer | 100 | 0.3019 | Anti V5 HRP | 0.045 |
| F10 scFv dimer | 100 | 0.0979 | Anti V5 HRP | 0.045 |
| F11 scFv dimer | 100 | 0.2042 | Anti V5 HRP | 0.045 |
| F12 scFv dimer | 100 | 0.1 | Anti V5 HRP | 0.045 |
| F13 scFv dimer | 100 | 0.1797 | Anti V5 HRP | 0.097 |
| F14 scFv dimer | 100 | 0.21470 | Anti V5 HRP | 0.097 |
| F15 scFv dimer | 100 | 0.1168 | Anti V5 HRP | 0.097 |
| F16 scFv dimer | 100 | 0.2144 | Anti V5 HRP | 0.097 |
| F17 scFv dimer | 100 | 0.1145 | Anti V5 HRP | 0.097 |
| B3 scFv dimer | 100 | 0.0976 | Anti V5 HRP | 0.1110 |
| C9 scFv dimer | 100 | 0.0985 | Anti V5 HRP | 0.0001 |
| VAR2CSA | 100 | 0.6314 | Anti V5 HRP | 0.045 |

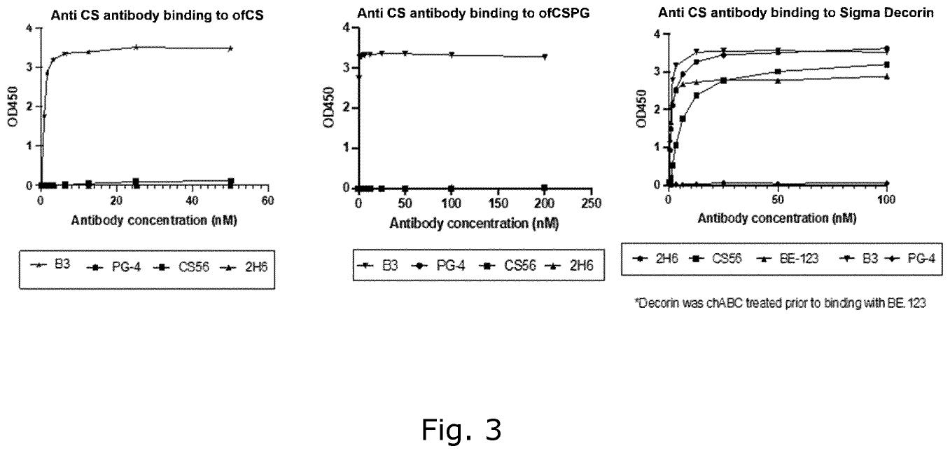

We then procured all available monoclonal antibodies previously described to bind chondroitin sulfates and tested binding to our ofCS, recombinant ofCSPG (ofCS on a decorin backbone) and purified decorin from Merck (Sigma-Aldrich). We tested the PG4, CS56, 2H6 and BE-123, with the latter being chondroitinase-treated as this antibody only recognizes a digested CS stump. For comparison, we included the ofCS binding B3 clone. Very clearly, we demonstrate that the previously described antibodies did not bind ofCS nor CSPG but did bind CS when presented on purified decorin (except PG4 that did not bind any of the coated CS reagents) (FIG. 3). This exemplifies that our panel of antibodies that bind ofCS and recombinant CSPG are unique compared to published antibodies. Further, these antibodies were all of the IgM type (Except BE-123 being an IgG, which in turn required enzymatic pre-digestion).

Having defined the onco-fetal specificity of the antibodies we next determined the preference towards distinct sulfations along the oncofetal CS chain modification. The table below shows the fine specificity of the panel of these preferred antibody fragments, either from binding inhibition assays or disaccharide analyses after pull down.

| | | | | From disaccharide | | | |
|---|---|---|---|---|---|---|---|
| | from GAG | | | analysis of pICS pool down | | | |
| | inhibition ELISA | | | | | | UA2S- |
| | CSA | CSB | CSC | GalNAc4S | GalNAc6S | GalNAc | GalNAc6S |
| F1 | ++ | +++ | + | | | | |
| F2 | + | +++ | + | | | | |
| F3 | + | 0 | +++ | + | ++++ | + | |
| F4 | ++ | ++ | + | | | | |
| F5 | + | +++ | + | | | | |
| F6 | + | +++ | + | | | | |
| F7 | + | +++ | + | | | | |
| F8 | + | 0 | ++++ | + | ++++ | + | + |
| F9 | + | + | + | | | | |
| F10 | + | + | + | | | | |
| F11 | + | ++++ | + | ++++ | + | | |
| F12 | + | + | + | | | | |
| F13 | + | + | + | | | | |
| F14 | + | + | + | | | | |
| F15 | + | + | + | | | | |
| F16 | + | + | + | | | | |
| F17 | + | + | + | | | | |
| B3 | + | +++ | 0 | | | | |
| C9 | ++ | + | ++ | +++ | +++ | ++ | |

Clearly F3 and F8 have a preference for 6 sulfation (CSC) whereas C9 is a mixed specificity. B3 and F11 and others appear to bind preferentially a dermatan sulfate containing GAG.

### Example 4

Determination of ofCS Antibody Affinities to ofCS Using a Biosensor

The Attana biosensor system has previously been proven very useful in measuring protein:carbohydrate interactions. To determine the kinetics and the exact affinity Kd of the ofCS antibodies to CS we thus used the Attana Biosensor platform (Attana A200, Attana AB) using a dual channel system. Briefly the sensor chip was coated with recombinant streptavidin (50 μg/ml) using EDC/S-NHS according to the manufacturer's protocol. Following, biotinylated ofCSPG was flushed over the A-channel chip giving a baseline shift indicating binding. The chip in the B-channel was left blank as control. A baseline was stabilized by passing running buffer (PBS) over the chip. Increasing antibody fragment concentrations (3.125-100 nM) were passed over the chips with a flow rate of 20 μl/min. The non-specific binding was subtracted using parallelly run non-coated chip. The chip was regenerated in between sample runs and data was analyzed using Attester Evaluation (Attana) and/or TraceDrawer (Ridgeview) software. On and off rates on the chip were used to evaluate the Kd-values and are plotted in Table 5.

TABLE 5
ofCS antibody affinity to ofCS

| | On rate | Off rate | Kd | Bmax at 400 nM |
|---|---|---|---|---|
| Construct | (M⁻¹s⁻¹) | (s⁻¹) | (nM) | dimer (Hz) |
| F1 (scFv2)* | 9.1 · 10⁴ | 4.0 · 10⁻⁴ | 4 | 80 |
| F2 (scFv2)* | 1.2 · 10⁵ | 5.7 · 10⁻⁴ | 5 | 50 |
| F3 (scFv2)* | 1.3 · 10⁵ | 3.1 · 10⁻⁴ | 2 | 50 |
| F4 (scFv2)* | 9.1 · 10⁴ | 6.8 · 10⁻⁴ | 7 | 45 |
| F5 (scFv2)* | 9.9 · 10⁴ | 3.4 · 10⁻⁴ | 4 | 50 |
| F6 (scFv2)* | 9.3 · 10⁴ | 2.7 · 10⁻⁴ | 3 | 80 |
| F8 (scFv2)** | 5.9 · 10⁴ | 6.0 · 10⁻⁴ | 10 | 180 |
| F9 (scFv2)** | 7.1 · 10⁴ | 2.8 · 10⁻⁴ | 4 | 180 |
| F10 (scFv2)** | 1.5 · 10⁴ | 2.5 · 10⁻⁴ | 17 | 140 |
| F11 (scFv2)** | 4.1 · 10⁵ | 6.3 · 10⁻⁴ | 1.5 | 210 |
| F12 (scFv2)** | 8.3 · 10⁴ | 5.1 · 10⁻⁴ | 6 | 110 |
| F13 (scFv2)** | 5.8 · 10⁴ | 5.0 · 10⁻⁴ | 8 | 170 |
| C9 (scFv2)* | 1.4 · 10⁵ | 9.4 · 10⁻⁴ | 7 | 50 |
| B3 (scFv2)* | 1.2 · 10⁶ | 9.2 · 10⁻⁴ | 1 | 80 |
| rVAR2** | 3.1 · 10⁵ | 6.5 · 10⁻⁴ | 2 | N/A |

*Fitted 1:2
**Fitted 1:1

In summary, all the tested phage display-derived antibody fragments showed very high affinity to ofCSPG in the low nanomolar range. Similar high affinity and high specificity to ofCS was seen with other antibody formats. This is a striking finding as carbohydrate interactions are usually of low affinity and low specificity.

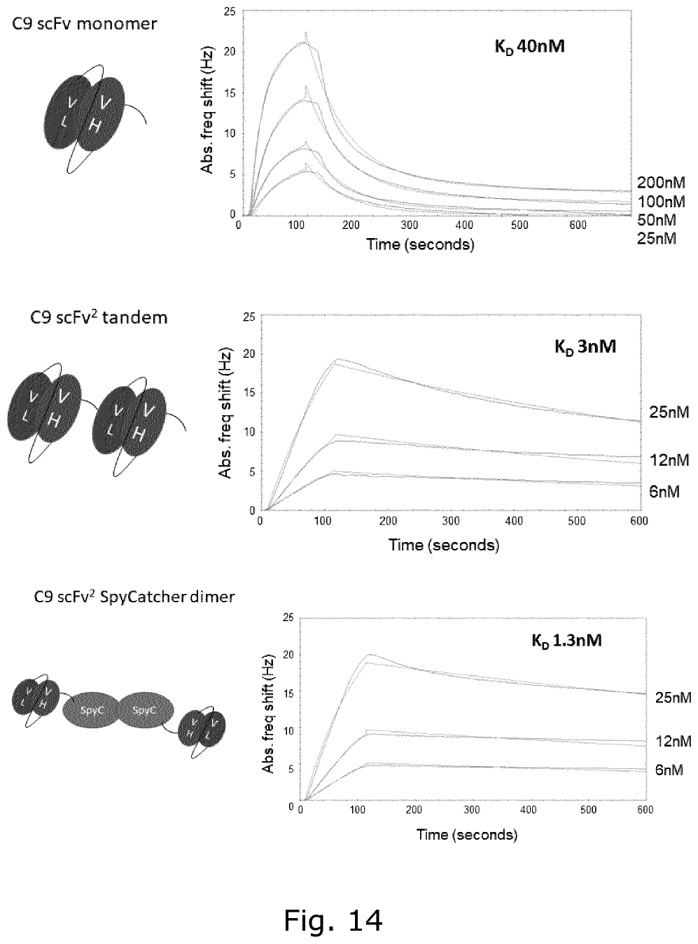

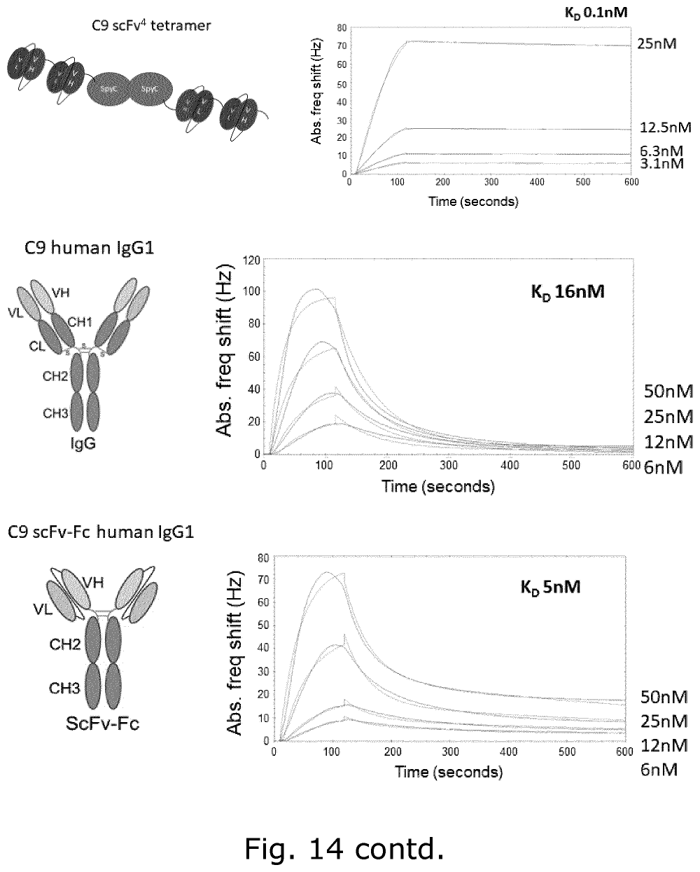

To test such other formats we constructed monomers, dimers and tetramers either through genetic fusion or conjugation to bivalent spycatcher formats through spytag. We also constructed full IgGs and scFV-Fc IgGs. All formats bound to ofCS with very high affinity between 0.1 nM to 40 nM (FIG. 14). Multimeric constructs are more effective binders than the corresponding monomers, which have been shown to exhibit K_{D} values of about 25 nm; the same figure also demonstrates that on-rates and off-rates can be varied depending on the choice of construct format. To be more exact, experiments designed to determine the K_{D} values for binding to ofCS by different formats of antibodies and variants of C9 (i.e. those antibodies and variants that have the light and heavy chain CDRs set forth in SEQ ID NOs: 121-127) have provided the following results:
C9 scFv monomer: K_{D}=40 nM
C9 scFv² tandem dimer: K_{D}=3 nM
C9 scFv² SpyCatcher dimer: K_{D}=1.3 nM
C9 IgG1: K_{D}=16 nM
C9 ScFv-Fc: K_{D}=5 nM
C9 scFv⁴ SpyCatcher tetramer: K_{D}=0.1 nM

Hence, depending on the specific intended use of the antibodies and antibody variants of the present invention, their binding affinities can be manipulated by selecting a suitable format. In the context of treatment or in vivo imaging for diagnosis of malignancies, targeting of a solid tumor with antibodies and antibody variants can benefit from the use of low molecular weight constructs having a high on rate and high off rate (since the target density is high in the solid tumor, the off-rate less critical compared to low abundance targets), whereas targeting of a non-solid malignancy (such as a leukemia), a construct having high affinity with a low off-rate will be more relevant. In addition, the various antibody and antibody variant formats differ in terms of their biological half-life—where it can be highly relevant to have a prolonged biological half-life in a number of therapy settings where a tumor is targeted, the opposite may be true in a diagnostic setting or for certain therapies including radiotherapy. Finally, when utilizing the antibodies and antibody variants in vitro in various assays, the fine-tuning of avidity of the constructs can be tailored to meet the exact need of the assay. In other words, the present invention allows for tailoring of the antibody-derived constructs to ensure that they are particularly suited for a specific task in terms of their affinity/avidity characteristics as well as in terms of their biological half-life.

### Example 5

ofCS Antibodies and Antibody Fragments Bind to a Wide Range of Cancer Cells In Vitro

We have previously shown that the malaria protein rVAR2, which binds ofCS, also bound to ofCS present on all cancer cell lines with no binding to normal cancer cells. The aim of making ofCS specific antibodies was to generate reagents with similar or overlapping specificities as rVAR2, i.e., binding to either cancer cell expressed ofCS or secreted ofCS.

Cells were grown to 70%-80% confluency in appropriate growth media and harvested in an EDTA detachment solution (Cellstripper). Cells were incubated with protein (300-25 nM) in PBS containing 2% fetal bovine serum (FBS) for 30 min at 4° C. and binding was analyzed in a FACSCalibur (BD Biosciences) after a secondary incubation with an anti-V5-FITC antibody. For inhibition studies, the protein was co-incubated with 400 μg/mL CSA (Sigma, Saint Louis, MO, USA; Cat #27042).

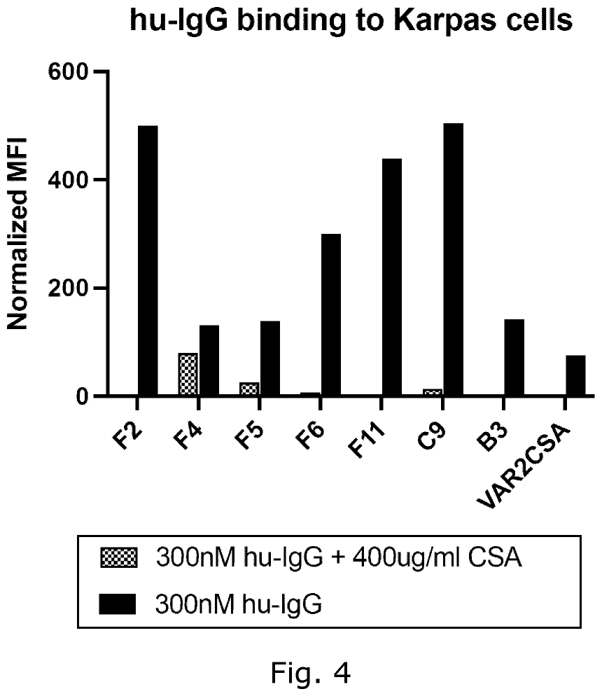

The data exemplified by binding of ofCS IgG antibodies to Karpas lymphoma cells tested in flow cytometry clearly showed that ofCS IgGs bound to a various extent to cancer cells (FIG. 4), and that the binding was either (most cases) fully or partly inhibited by adding soluble chondroitin sulfate. Binding was in all cases higher than with rVAR2, which however also could be attributed to the use of different secondary antibodies, with rVAR2 being tested with an anti-V5 antibody and the monoclonal antibodies with an anti-human IgG.

We then expanded the analyses to include the scFv formats, this time tested on a solid breast cancer tumour cell line (4T1). rVAR2 was included as a benchmarking control. For all antibody fragments we showed a significant tumour binding (Table 6), in many cases many-fold higher than with rVAR2, and in all cases many-fold higher than with the antibody control alone, with the exception of F3 only showing 2-fold increase compared to control.

TABLE 6
ofCS scFv binding to 4T1 breast cancer cells

| | | | | Antibody | Autofluo- |
|---|---|---|---|---|---|
| | Concentration | 4T1 cells | Detection | control | rescence |
| Protein | (nM) | (MFI) | antibody | (MFI) | (MFI) |
| F1 scFv dimer | 150 | 1417 | Anti HIS FITC | 140 | 110 |
| F2 scFv dimer | 150 | 2652 | Anti HIS FITC | 140 | 110 |
| F3 scFv dimer | 150 | 270 | Anti HIS FITC | 140 | 110 |
| F4 scFv dimer | 150 | 6190 | Anti HIS FITC | 140 | 110 |
| F5 scFv dimer | 150 | 4261 | Anti HIS FITC | 140 | 110 |
| F6 scFv dimer | 150 | 1911 | Anti HIS FITC | 140 | 110 |
| F7 scFv dimer | 150 | 627 | Anti HIS FITC | 140 | 110 |
| F8 scFv dimer | 150 | 1958 | Anti V5 FITC | 94 | 85 |
| F9 scFv dimer | 150 | 13206 | Anti V5 FITC | 94 | 85 |
| F10 scFv dimer | 150 | 10407 | Anti V5 FITC | 94 | 85 |
| F11 scFv dimer | 150 | 11798 | Anti V5 FITC | 94 | 85 |
| F12 scFv dimer | 150 | 9373 | Anti V5 FITC | 94 | 85 |
| F13 scFv dimer | 150 | 2053 | Anti V5 FITC | 94 | 85 |
| F14 scFv dimer | 150 | 14306 | Anti V5 FITC | 94 | 85 |
| F15 scFv dimer | 150 | 12363 | Anti V5 FITC | 94 | 85 |
| F16 scFv dimer | 150 | 8192 | Anti V5 FITC | 94 | 85 |
| F17 scFv dimer | 150 | 6372 | Anti V5 FITC | 94 | 85 |
| B3 scFv dimer | 150 | 1335 | Anti His FITC | 140 | 110 |
| C9 scFv dimer | 150 | 2997 | Anti HIS FITC | 140 | 110 |
| VAR2CSA | 150 | 4394 | Anti HIS FITC | 140 | 110 |

Again, we wanted to benchmark our panel of ofCS antibodies to the available and published antibodies CS56, 2H6, and BE-123. For comparison we included B3 IgG. The CS56 and 2H6 antibodies only show marginally increased binding over the negative control, compared to B3, which demonstrated a more than 100-fold higher binding than the control (Table 7). The BE-123 again required chondroitinase treatment to show binding.

TABLE 7
ofCS antibody and commercial CS antibody binding to Karpas cancer cells

| | | | | Antibody | Autofluo- |
|---|---|---|---|---|---|
| | Concentration | Karpas cells | Detection | control | rescence |
| Protein | (nM) | (MFI) | antibody | (MFI) | (MFI) |
| B3 hu-IgG | 200 | 10463 | Anti hu-IgG FITC | 97 | 67 |
| CS56 | 200 | 131 | Anti IgM FITC | 89 | 67 |
| 2H6 | 200 | 252 | Anti IgM FITC | 89 | 67 |
| BE-123 | 200 | 1461 | Anti mu-IgG FITC | 111 | 67 |

We then tested selected scFvs on a larger panel of diverse tumour cells. In the analyses we included fresh white blood cells from a healthy donor. The flow cytometry values were scored, and a value between 0-1 is background and similar to a negative antibody control, whereas values at 2 or above are considered as positive binding. It was evident that the ofCS antibodies bind to diverse tumour cells with no binding to normal white blood cells.

TABLE 8
ofCS antibody binding to different cancer cell lines and white blood cells

| | Karpas | Colo205 | PC3 | Sw80 | 4T1 | HT29 | A375 | White blood cells |
|---|---|---|---|---|---|---|---|---|
| Protein | (lymphoma) | (colorectal) | (Prostate) | (Colorectal) | (Mu-Breast) | (Colorectal) | (Melanoma) | (Healthy ctrl) |
| F1 | 2 | 2 | 2 | 2 | 2 | 3 | 0 | 0 |
| F2 | 6 | 6 | 3 | 4 | 5 | 3 | 7 | 1 |
| F4 | 10 | 10 | 10 | 10 | 11 | 10 | | 1 |
| F5 | 4 | 4 | 5 | 2 | 8 | 5 | 6 | 0 |
| F6 | 2 | 2 | 5 | 2 | 5 | 2 | 5 | 0 |
| F8 | 10 | | 4 | 5 | 1 | | 3 | |
| F9 | 10 | | 14 | 11 | 20 | | 7 | |
| F10 | 10 | | 11 | 7 | 13 | | 7 | |
| F11 | 11 | | 11 | 9 | 13 | | 9 | |
| F12 | 9 | | 12 | 7 | 13 | | 8 | |
| F13 | 7 | | 8 | 6 | 8 | | 7 | |
| F14 | 7 | | 13 | 8 | 2 | | 9 | |
| F15 | 9 | | 14 | 8 | 17 | | 9 | |
| F16 | 7 | | 14 | 8 | 20 | | 8 | |
| F17 | 6 | | 7 | 4 | 8 | | 6 | |
| B3 | 9 | 9 | 5 | 8 | 5 | 1 | 7 | 0 |
| C9 | 10 | 10 | 5 | 4 | 8 | 1 | 10 | 1 |
| VAR2 | 10 | 9 | 5 | 10 | 9 | 10 | 8 | 1 |

### Example 6

ofCS Antibody Fragments Bind to Tumour and Placenta Tissue Sections with No Binding to Non-Cancer Tissues

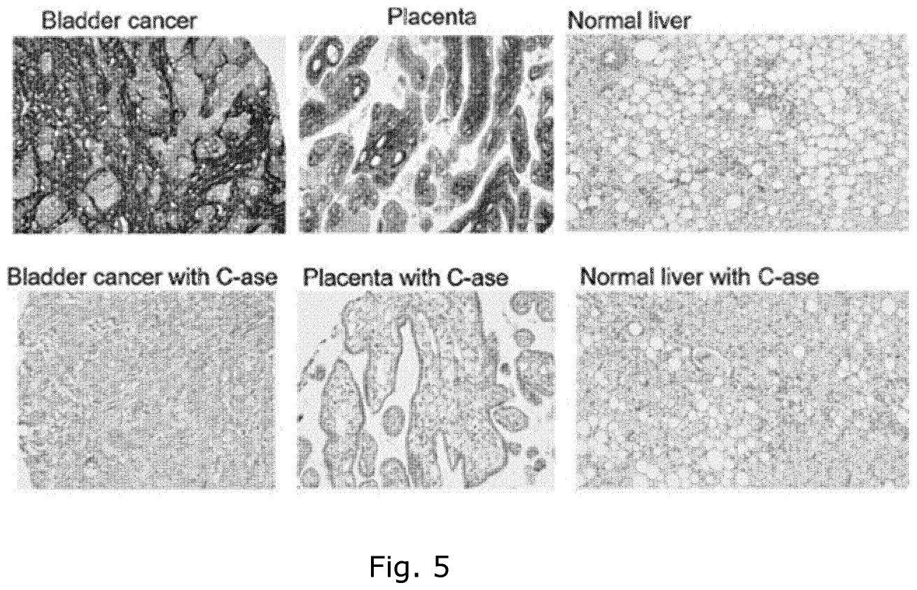

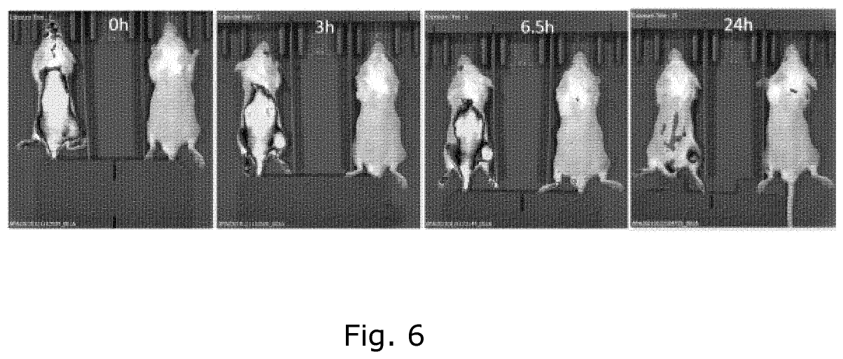

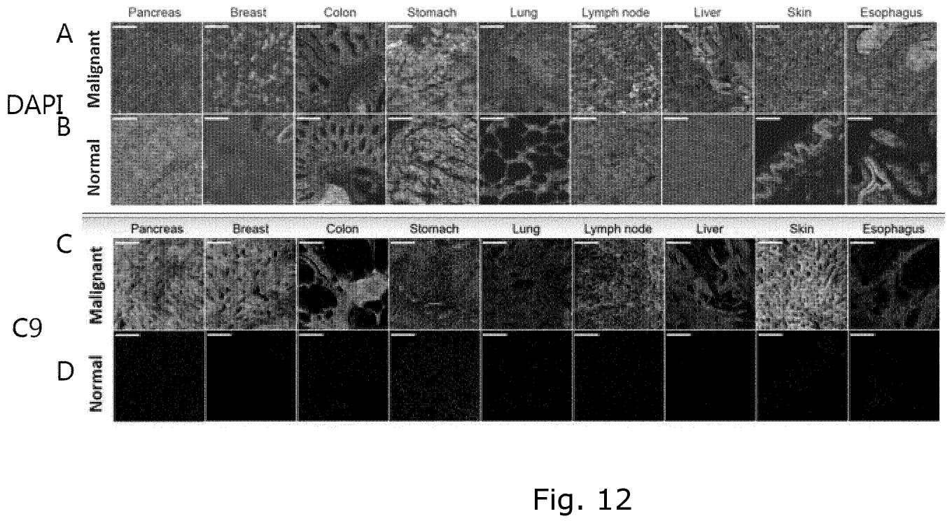

The binding of the antibody fragments to primary cancer tissue obtained from human patients was investigated using immunohistochemistry (IHC). The staining protocol was optimized on the Ventana Discovery XT platform with no epitope retrieval. Paraffin embedded tissue spotted on glass slides was incubated with V5 tagged scFv for 1 h in room temperature, washed for 8 minutes, incubated with 1:700 mouse anti-V5 antibody for 30 minutes, washed for 8 minutes. Bound anti-V5 was subsequently detected using UltraMap anti-mouse HRP. All the antibody fragments stained tumour tissue, exemplified here with bladder cancer tissue binding, further the antibodies bound to ofCS in placenta tissue, and there was no binding to non-cancer tissue, exemplified here with liver tissue (FIG. 5). The staining could be blocked by adding 200 μg/μl CSA or by chondroitinase ABC treatment (data not shown). To demonstrate the capacity of the antibodies and antibody variants to stain all types of tumor tissue, we stained a tissue microarray containing malignant tissue and corresponding normal adjacent tissue from Pancreas, Breast, Colon, Stomach, Lung, Lymph node, Liver, Skin and esophagus and detected binding using immunofluorescence. To visualize the integrity of the tissues all sections were stained for nuclei with DAPI. FIG. 12 shows the binding using the C9 scFv antibody fragment and shows strong binding to all the malignant tumor tissues with no binding to normal non-malignant tissue.

### Example 7

### IVIS Tumour Localization Data

Anti-ofCS IgGs were NIR labeled through available cysteines with an Alexa750 using maleimide chemistry. This was done with an excess of NIR probe (4× molar) according to the manufacturer's instructions. The coupled protein was injected (4 mg/kg) IV in the tail vein of healthy and tumour bearing mice 10 days post-establishment of a subcutaneous tumour in the right flank. The mice were scanned using an IVIS spectrum CT scanner (Perkin Elmer). Scanning was done at time intervals ranging from 10 min to 48 hr. In vivo tumour signal quantification is presented as an absolute signal in reference to the signal of the flank of the healthy control mouse. Data analysis was performed using the Living Image Software (Caliper Life Sciences). We found that the anti-ofCS IgG located to the tumour after a few hours and remained measurable for a minimum of 24 hours (FIG. 6).

### Example 8

Toxin-Conjugated ofCS IgG—In Vitro Cancer Cell Killing

To address if the antibodies can be used to deliver a cytotoxic payload to cancer cells, we prepared antibody-drug conjugates (ADC) according to standard procedures by conjugating a vc-MMAE microtubule inhibitor to cysteine on the IgG. In brief, we added TCEP to reduce IgG disulfide bonds at 3-6 molar excess and incubated at 37° C. for 90 min. The reaction was cooled down and 8 molar excess of Mal-vc-MMAE was added and incubated for 60-90 min at +4° C. The reaction was stopped by adding 8 molar excess cysteine and incubating 15 min. Free Mal-vc-MMAE was removed using a Zeba spinn column equilibrated in PBS followed by up-concentration with a Viva spinn column. The ADC were quality controlled by SDS-page and HPLC-SEC and the DAR was determined in the range of 2-4. Further the ADCs were post conjugation demonstrated to retain tumour cell binding by flow cytometry as well as ofCS binding in ELISA. Cells were removed from their culture vessel using Gibco® Trypsin-EDTA (Invitrogen #25300-054). Detached cells were diluted in respective growth medium (Invitrogen #: 10313-021, A10491-01, 16600-082, 12561-056, 35050-061, 11415-064)+10% Fetal bovine serum (Corning #: 35-015-CV) to 25,000 cells/mL such that 100 μl/well will dispense 2500 cells/well. Cells were seeded into black walled, flat bottomed 96-well plates (Costar #3904). Adherent cell line cells were incubated for one night at 37° C. in a 5% CO₂ atmosphere to allow the cells to attach to the microtiter plate surface, while suspension cells were seeded immediately before use. Test compounds were diluted directly in the appropriate cell growth medium at five-times the desired final concentration. These compounds were then titrated 1:3, over eight steps. A control with no test compound present (growth medium alone) was included on each microtiter plate in sextuplicate. 25 μl/well of the prepared titrations was added in triplicate to each cell line assayed. The cells and titrations were incubated at 37° C./5% CO₂ for five nights. After the incubation, cell viability was measured using CellTiter-Glo® (Promega #G7572) reagent by adding thirty μl of prepared CellTiter-Glo® to each assay well. The mixtures were incubated for at least twenty min in the dark prior to measuring emitted luminescence using a microplate luminometer (500 ms integration time). The collected relative luminescence units (RLU) were converted to % cytotoxicity using the RLU values measured from the growth medium alone control as follows: % Cytotoxicity=1−[Well RLU/average medium alone control RLU]. Data (% Cytotoxicity vs. Concentration of ADC (log 10 [nM]) were plotted and were analyzed by non-linear regression methods using GraphPad Prism software v. 5.02 to obtain EC50 estimates.

All tested antibodies specific to chondroitin sulfate and conjugated with a toxin mediated cellular killing with low nanomolar IC50 values, identical to previous published results using rVAR2 drug conjugates. This was demonstrated on both murine cancer cells (4T1) and a panel of human cancer cells including PC3 prostate, Karpas lymphoma, Colo205 colorectal as well as fresh patient derived cell lines (PDX). Table 9 shows representative ADCs tested on the PC3 prostate cancer cell line and the 4T1 breast cancer cell line. As a negative control an ADC not binding to cancer cells was used and as a positive control, we used free soluble MMAE, which readily diffuses inside the cells and mediates cell killing. VAR2CSA drug conjugate (VDC) was used for benchmarking.

TABLE 9
In vitro cytotoxicity of MMAE-conjugated
ofCS IgG on 4T1 and PC3 cancer cells

| Protein | IC50 4T1 | IC50 PC3 |
|---|---|---|
| ADC-F3 | 30 | 9 |
| ADC-F4 | 2 | 1 |
| ADC-F9 | 44 | 3 |
| ADC-Ctrl | 463 | 22 |
| VDC | 49 | 2 |
| Free MMAE | 1.4 | 0.2 |

The drug conjugates' ability to mediate cancer cell killing will further be tested on a panel of cancer cell lines, representing all types of tumour origin (i.e., lines of hematopoietic, epithelial, and mesenchymal origin).

### Example 9

Toxin-Conjugated ofCS IgG—In Vivo Tumour Cell Killing

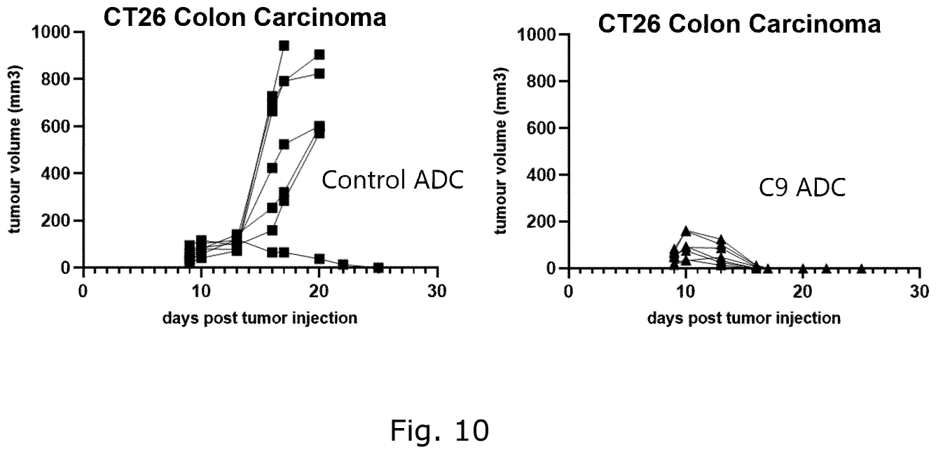

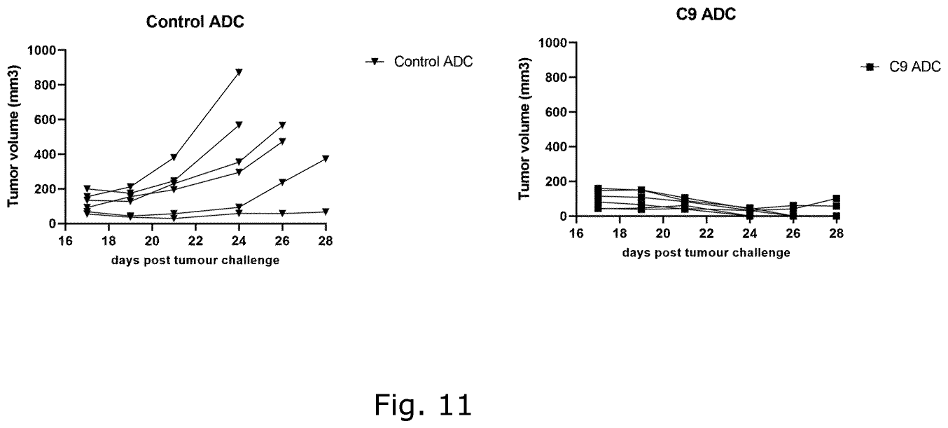

A cancer therapy based on targeting ofCS with our panel of antibodies could be in the format of antibody drug conjugates. To demonstrate efficacy with an anti-ofCS ADC we established animal models of murine colon carcinoma by injecting murine CT26 tumour cells (100.000 cells) subcutaneously into the right flank of Balb/c immunocompetent mice. To test efficacy also against human tumors we established Karpas lymphoma (1·10⁶ cells) in SCID mice. When the tumours in both models reached a minimum size of 70-120 mm³, the mice were randomly divided into 3 groups, and treated with intravenous injections of vehicle (saline), a control ADC, or an ofCS ADC, respectively, at 3 mg/kg doses 3 times in total. Tumour growth was monitored using a caliper-measuring tool, and the three longest perpendicular axes in the x/y/z plane of each tumour was measured. Tumour volume was calculated according to the standard formula: volume=xy2×0.5236. The mice were weighed three times a week to monitor acute toxic effects. In CT26 colon cancer model there was complete tumor regression in all treated animals, whereas 6/7 mice in the control group reached their endpoint with large tumors at day 20 (see FIG. 10). The ofCS ADC treated mice remained tumor free throughout the entire study (4 months) and were resistant to challenge with new tumors. Similar for Karpas (see FIG. 11), all mice showed regression and 4/6 mice were cured. All animals in the treatment group gained weight during the study and did not show any signs of acute or long-term toxicity.

On this background, it is expected that the treatment regimen to be well tolerated also in humans and effective, with a complete stall of tumour growth or regression of the tumour size after treatment.

### Example 10

ofCS Antibodies Hinder Cancer Cell Migration and In Vivo Tumour Seeding and Metastatic Spread

We wanted to see whether targeting ofCS with our antibodies would interfere with tumour metastasis in vivo. For this purpose, we established two animal models exploring two essential events of metastatic spread; cell settlement (or seeding) and tumour implementation. For the 4T1 seeding model, 5-105 luciferase-marked cells suspended in 100 μL of 100 nmol/L of ofCS specific antibody or saline solution or isotype matched control antibody were injected into the tail vein of immunocompetent mice. Animals were monitored until 7 weeks after injection using the IVIS imaging system. Mice were sacrificed when they reached the predefined humane end point. For the B16 melanoma model (tumour implementation model), 5-10⁵ B16-F10GP cells in 100 μL PBS were injected into the right flank of C57BL/6 mice. The animals were randomized into two groups of 10 mice. One group was treated by intravenous injection of 100 μg ofCS antibody at days 0, 6, and 9. The control group was treated with equal volume PBS and another control group was treated with an isotype specific control antibody. Tumour size was monitored by manual measurements using a caliper-measuring tool, taking measurements at the two longest perpendicular axes in the x/y plane of each tumour. Tumour volume was calculated according to the standard formula: volume=xy2×0.5236.

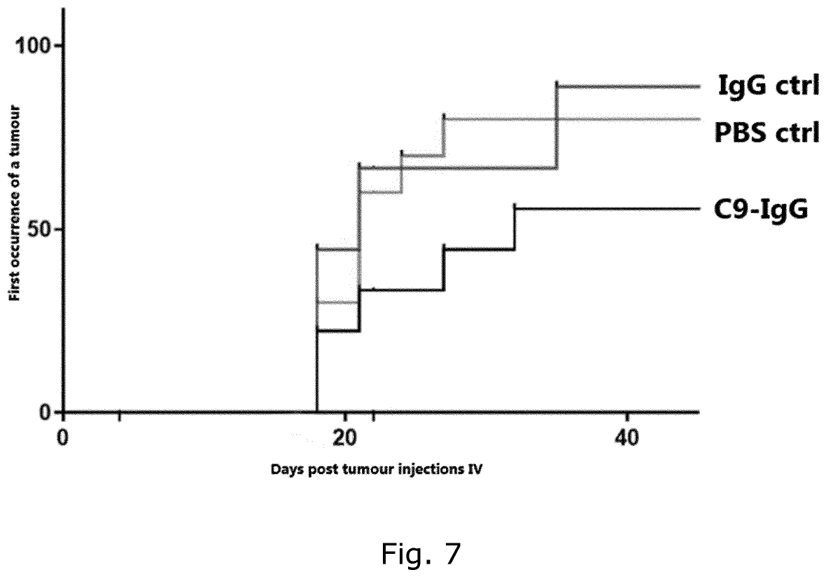

Preincubating 4T1 breast cancer cells with ofCS antibodies strongly inhibited settlement in distant organs and significantly prolonged lifespan of the treated mice (FIG. 7). The same was evident for treatment of subcutaneous B16 melanoma tumours at time of implementation. Combined with our data demonstrating that ofCS antibodies bind to circulating tumour cells (see Example 14), it is very likely that treating patients with antibodies that bind and opsonize cancer cells in circulation will have significant clinical impact.

Furthermore, we plan to test the effects of ofCS antibodies on cancer cell migration and invasion in vitro. We will do this by growing tumour cells of various origin to 70% confluency. Then they will be serum-starved in the presence of 450 nmol/L ofCS antibody or control antibody for 24 hours. The cells will be dislodged with Cellstripper and counted 3 times. Then, 100,000 cells will be added to each insert of a Boyden chamber plate (Chemi-Con). Separate kits will be used for migration and invasion. The invasion kit includes membranes coated in basement membrane extract. Media with or without chemoattractant will be added to the lower well. Plates will then be incubated for 18 to 36 hours at 37° C. The number of migrating cells will be determined by a fluorescent probe and compared to a standard curve. We expect to see that the ofCS antibodies interfere with cell migration. This would support a key biological role of the ofCS antibodies, similar to the proposed mechanism of rVAR2 inhibition of cell migration through impairing focal adhesion pathways.

### Example 11

ofCS Targeting CAR T Cells Stop Tumour Growth In Vitro and In Vivo

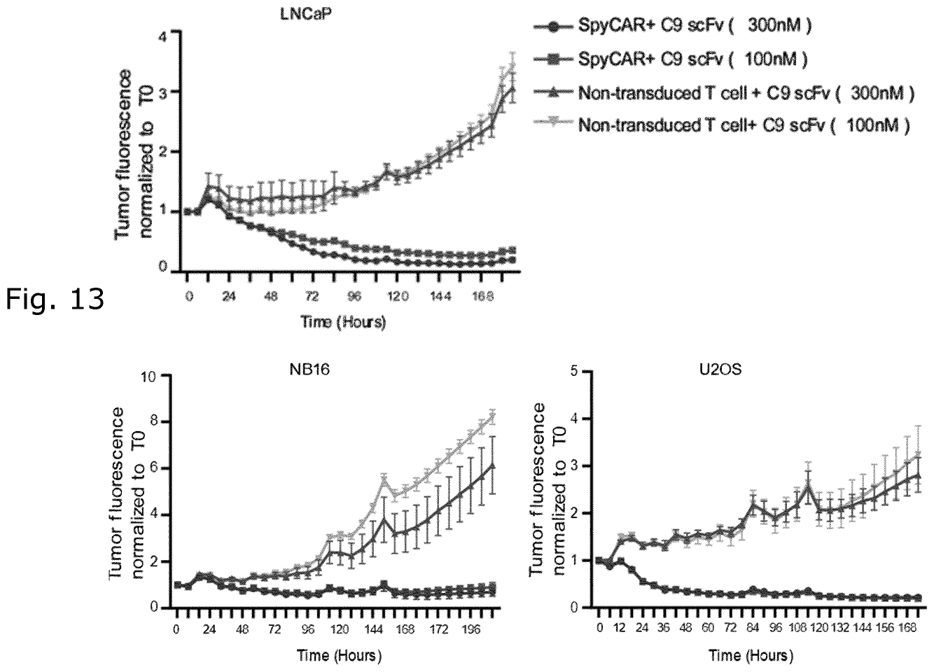

Chimeric antigen receptor (CAR) T cells can be modified to either express an anti-ofCS antibody fragment or present a SpyCatcher protein for conjugation of a spytagged scFV fragment. Here we transduced human T cells (immune effector cells) with a CAR comprised of a spycatcher domain, CD28 and/or CD3 zeta signaling domain, and a signalling peptide so that the CAR portion of the construct can be glycosylated and anchored in the cell membrane of the immune effector cell. CAR expressing T cells (CAR T cells) were then mixed with a spytagged scFV against ofCS (here the scFV is the C9 sequence) and mixed with human cancer cells such as LNCap (Prostate cancer), U2OS (osteosarcoma) and monitored for survival using IncuCyte instrumentation. In all cases the ofCS targeting CAR-T cells were able to effectively kill tumor cells in vitro, and notably tumor cells of very different origin. The negative control being non-transduced T-Cells mixed with ofCS antibody fragment did not have an effect on tumor cells. See FIG. 13.

On the background of the findings reported herein, it is expected that an effect in animal efficacy studies will be demonstrated.

### Example 12

Immunocytokines: An Immunotherapy Based on ofCS mAb Fused to a Cytokine

We would like to generate immunocytokines consisting of an ofCS mAb fused to a cytokine, which can simultaneously target a tumour and stimulate an anti-tumour immune response. To this end, human and murine cancer cells (e.g., MG-63, RH-30, K7M2, NB-16) stably expressing fluorophores for tracking purposes will be co-cultured with peripheral blood mononuclear cells (PBMC) and treated with ofCS-targeting scFv fragments fused to a variant of the immune stimulatory cytokine interleukin-2 (ofCSscFv-IL2v). Specifically, we want to develop a novel monomeric bispecific ofCS-targeted immunocytokine that comprises an IL-2v moiety with decreased IL-2 receptor a (CD25) binding. Cancer cell survival and PBMC proliferation will be monitored in real-time using IncuCyte instrumentation. ofCSscFv-IL2v performance will be tested in conventional cultures (2D) and spheroid models that more closely resemble the 3D organization of tumour tissues. Compared to commercially available recombinant wildtype IL-2, we expect to see a superior ability of ofCSscFv-IL2v to kill cancer cells of various origin. In another iteration, we will test the ability of ofCSscFv-IL2v to activate PBMCs through evaluating PBMC proliferation and cytokine secretion similarly to equimolar concentrations of IL-2v. In addition, we want to compare the cancer cell binding features of ofCSscFv-IL2v with those of ofCSscFv. In another iteration, we want to test the systemic anti-tumour protection in vivo by intratumour administration of ofCSscFv-IL2vtumour. Furthermore, we will test if a potential anti-tumour effect can be boosted by synergetic effects with immune checkpoint blockade (anti-PD-L1/PD-1 or anti-CTLA4).

### Example 13

An Immunotherapy Based on ofCS mAb Fused to antiCD3 Eradicates Tumours In Vivo

This study was done to test whether scFv fragments of the ofCS antibodies fused to a murine anti-CD3 antibody could cure cancer in mice with a fully functioning immune system. The murine cell lines 4T1 (breast cancer), B16-F10 (melanoma), CT26 (colorectal cancer), and TC-1 (primary lung epithelial cell-derived) were used for in vitro and in vivo experiments, in the latter injected into either C57BL/6J or BALB/c AnNRJ (BALB/c) mice. Mice were either completely randomly assigned to a group or separated into each group based on size for an equal mean of tumour size before treatment. For all experiments, we used C57BL/6J and BALB/c AnNRJ (BALB/c) mice purchased from Janvier labs. Mice were 6-8 weeks on arrival and kept for at least one week prior to use.

C57BL/6J mice were injected SC with 100,000 B16-F10 cells in 100 μL PBS in the lower left quadrant of the belly or in the right flank. BALB/c mice were injected with either 75,000 4T1 or 500,000 CT26 cells SC in 100 μL PBS in the right flank. All mice were treated peritumourally (PT) with 12 μg ofCSscFV-aCD3Mu in PBS in a total volume of 50 μL. 100 μg murine anti-mCTLA-4 (Invivofit) was administered three times intraperitoneally (IP) distributed over a week, with the first injection being double the dosis. 100 μg InvivoPlus anti-mouse PD-1 (BioXCell) was administered IP twice within the first week.

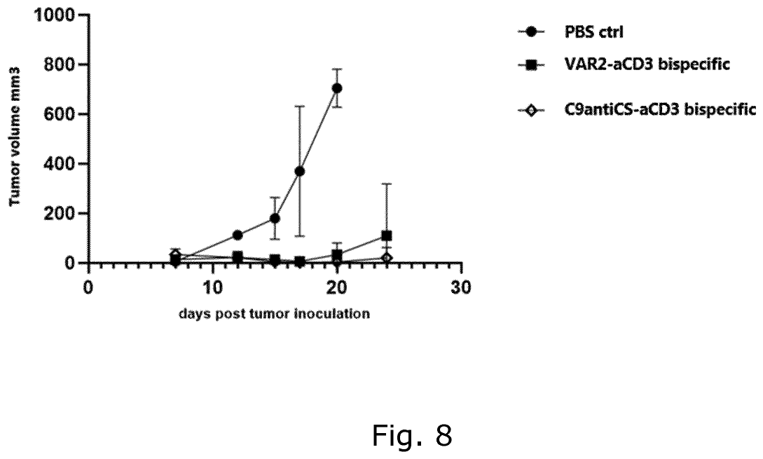

The ofCSscFV-aCD3Mu molecule displayed preserved binding of each moiety, as assessed by flow cytometry, and induced effective killing of cancer cells in vitro when adding pre-activated splenocytes. In vivo, ofCSscFV-aCD3Mu prevented establishment of murine breast cancer 4T1 tumours when starting treatment peri-tumourally (PT) on day 1 after tumour cell injection (FIG. 8). It was also able to slow growth of larger solid tumours. When combining ofCSscFV-aCD3Mu with a check-point inhibitor (CPI), the ofCSscFV-aCD3Mu treatment eliminated solid tumours and metastasis in the majority of the treated mice. To examine if this highly effective treatment regimen would be effective beyond 4T1 breast tumours, we established subcutaneous models of B16-F10 melanoma and CT26 colorectal tumours and treated with combinations of CPI and V-aCD3Mu. Also in these models, V-aCD3Mu and CPI induced complete tumour regression in most of the treated mice. In summary, our data suggest that the ofCS-targeted immunotherapies are effective against a wide range of tumours. This effect can be enhanced in combination with CPIs, results in complete responses, and induces an abscopal response that renders the mice resistant to tumour re-challenge.

### Example 14

### Specific Capture of Extremely Rare Cancer Cells in Blood Samples for Diagnostic Purposes

Background: Circulating tumour cells (CTCs) are malignant cells that have detached from solid tumour compartments and entered the lymph or blood circulation. While the presence of CTCs in the blood is associated with a poor patient prognosis, it offers an opportunity for continuous, non-invasive access to information regarding tumour evolution and progression. This could potentially enable early detection of cancer as well as provide the groundwork for more qualified treatment decisions. Unfortunately, CTCs are extremely rare compared to the vast number of normal white blood cells. Furthermore, CTCs are characterized by a high degree of heterogeneity and cellular plasticity, which complicates their specific and sensitive isolation.

Methods: In order to measure the binding of the various antibody constructs to cancer cells or healthy white blood cells (WBCs), flow cytometry analysis was applied. WBCs were isolated from freshly drawn blood from a healthy donor by a simple red blood cell lysis followed by a wash step. Colorectal cancer cells (COL0205) were harvested from a cell culture, and both cell types were incubated with 150 nM of the respective constructs. The scFv constructs containing a SpyTag were dimerized through a SpyCatcher-dimer containing a HIS tag and binding was detected using anti-HIS FITC antibody. To investigate whether the antibody constructs could successfully bind and enable the isolation of few cancer cells spiked into a blood sample, the constructs were dimerized using a biotinylated SpyCatcher-dimer (in case of full IgGs the antibodies were biotinylated directly). After carefully spiking 100 pre-stained cancer cells into 3 mL blood, the blood samples were processed by red blood cell lysis followed by incubation with ˜100 nM of the respective constructs. Finally, streptavidin coated magnetic beads were added allowing for high-affinity binding between the biotinylated antibody constructs and the magnetic beads. Using a simple magnet, antibody-bound cancer cells were separated from the vast number of healthy WBCs and counted by microscopy.

### Results

As seen in Table 10, the various constructs bound cancer cells with an at least 100-fold increased intensity compared to healthy WBCs. The ability of the antibodies to distinguish between the target cells (cancer cells) and non-target cells is extremely important, since patient blood samples may only contain a few CTCs in a background of millions of WBCs.

The high degree of specificity was further tested by coupling the F4 scFv to a SpyCatcher-dimer labeled with a strong fluorophore and using the construct for staining the cancer cells after spike-in to blood. When analyzing the sample by microscopy, a bright and clear staining of the COL0205 colorectal cancer cells was observed with little or no staining of the surrounding healthy white blood cells.

The ability of the antibody constructs to isolate cancer cells from a blood sample was also tested. In order to enable identification of cancer cells, these cells were pre-stained with a fluorescent dye. 100 cancer cells were spiked into 3 mL blood and processed as described above. Various types of cancer cells were tested. After antibody-based magnetic isolation of the cells, microscopy analysis revealed the number of cancer cells captured. The number of captured cells relative to the number of cells spiked into the sample constitutes the recovery (percentage), which is shown in Table 11. The antibody constructs successfully captured various types of cancer cells from blood.

TABLE 10
Mean fluorescent intensity in flow cytometry
(150 nM antibody construct)

| | Colo205 | White blood cells | Fold increase in |
|---|---|---|---|
| Protein | (Colorectal) | (Healthy) | cancer cell binding |
| F1 (scFv-dimer) | 167,082 | 1,611 | 104 |
| F2 (scFv-dimer) | 665,000 | 3,532 | 188 |
| F4 (scFv-dimer) | 1,090,000 | 3,487 | 313 |
| F5 (scFv-dimer) | 297,215 | 1,940 | 153 |
| C9 (scFv-dimer) | 1,320,000 | 2,427 | 544 |

TABLE 11
Recovery of 100 cancer cells spiked into whole blood

| | | | | |
|---|---|---|---|---|
| | | | | A549 |
| | Colo205 | A375 | C32 | (Lung |
| Protein | (Colorectal) | (Melanoma) | (Melanoma) | cancer) |

| | | |
|---|---|---|
| B3 | 76% | |
| (Full IgG) | | |
| C9 | 93% | 56% |
| (scFV-dimer) | | |
| F4 | 96% | |
| (scFV-dimer) | | |

Conclusion: In conclusion, a broad range of antibody constructs tested (scFv, Fab and full IgG) have shown the ability to specifically bind to cancer cells even when these target cells are found within an extremely high background of normal non-target cells such as white blood cells. This opens up the possibility of exploiting the antibody constructs for the capture of rare circulating tumour cells (CTCs) from cancer patient blood samples, thus providing a plethora of novel diagnostic opportunities.

### Example 15

### Specific Capture of Proteoglycans in Liquid Biopsies for Diagnostic Purposes

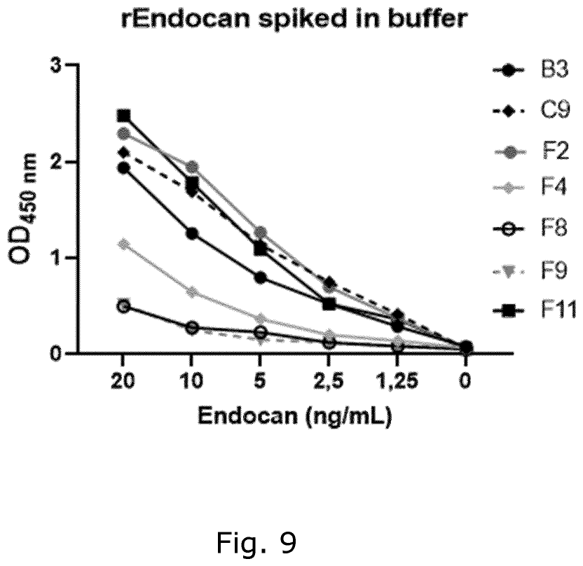

We have demonstrated that tumours overexpress ofCS modified proteoglycans. Mass spectrometry analyses of pulled down proteoglycans from urine, plasma and tumour biopsies using either VAR2 or anti-ofCS antibodies identify a wide range of of-CS modified proteins including but not limited to agrin, biglycans, CD44, decorin, glypicans, endorepellin, integrin beta-1, laminin subunit gamma-2, neuropilin-1, syndecans, testican, sushi repeat-containing proteins, CSPG4, endocan, and versican. Using combinations of VAR2, ofCS antibodies and/or antibodies specific for a proteoglycan protein core we have developed methods to detect CS-modified proteoglycans in bodily fluids such as blood plasma, cerebrospinal fluids and urine. In this Example, we immobilized anti-ofCS antibodies on ELISA plates. We then added endocan in different concentrations spiked into sample buffer and detected the levels of ofCS captured proteoglycan using an antibody specific for the protein core of the CSPG (FIG. 9).

Such assays have clinically relevant detection ranges down to 1 ng of endocan. In some cases, the plasma sample can be enriched for GAGs prior to analyses using an IEX column.

### Example 16

### Characterization of Binding Groove

The CS binding scFvs share a common feature, as they are predicted to have a positively charged binding groove as defined by alphafold structural prediction, similar to a positive groove pivotal for VAR2CSA binding to ofCS (Wang et al., Nat. Commun., 2021). The scFv binding groove is defined by the following criteria and applies to a structural class of antibodies with a degree of freedom in the variable loop sequences.
a) The CDR-L2 and CDR-H3, defined using the KABAT scheme, contain multiple lysine or arginine type amino acid residues. In addition, CDR-L1 and CDR-H2 can also contain lysine or arginine residues. In total, 3-6 residues in the class of scFvs analyzed are present and are surface exposed forming the positive CSA/CSC binding groove. In all cases, positive residues are present in both the VH and VL domain, creating a groove spanning across both variable domains.
b) The positive binding groove is defined by surface exposed arginine and lysine type amino acid residues in close proximity to each other. This proximity is defined by measuring the distance between the C-alpha residues. The shortest distance between two adjacent residues in space is between 7 and 10 Å. As an example, for C9 scFv the distance between C-alpha:C-alpha atoms for residue CDR-L3 ARG100:CDR-H3 ARG229 is 8.4 Å, CDR-H3 ARG229:CDR-H3 ARG227 is 7.0 Å, CDR-H3 ARG229:CDR-L2 ARG51 is 10.5 Å, and CDR-L2 R51:CDR VL LYS67 is 9.3 Å.

### LIST OF REFERENCES

Agerbæk, M. Ø. et al. 2018. The VAR2CSA malaria protein efficiently retrieves circulating tumor cells in an EpCAM-independent manner. Nat. Commun. 9(1):3279. doi: 10.1038/s41467-018-05793-2
Beaudet, J. M. et al. 2014. Characterization of human placental glycosaminoglycans and regional binding to VAR2CSA in malaria infected erythrocytes. Glycoconj. J. 31(2):109-16. doi: 10.1007/s10719-013-9506-6
Davies, L. C. et al. 2008. Chondroitin sulphate impedes the migration of a sub-population of articular cartilage chondrocytes. Osteoarthritis and Cartilage, 16(8):855-864. doi: 10.1016/j.joca.2007.12.005
Detamore, M. S. et al. 2005. Quantitative analysis and comparative regional investigation of the extracellular matrix of the porcine temporomandibular joint disc. Matrix Biol. 24(1):45-57. doi: 10.1016/j.matbio.2004.11.006
García-Piqueras, J. et al. 2019. Chondroitin sulfate in human cutaneous Meissner and Pacinian sensory corpuscles. Anat. Rec. (Hoboken). 302(2):325-331. doi: 10.1002/ar.23951
Hardingham, T. E. et al. 1994. The sulphation pattern in chondroitin sulphate chains investigated by chondroitinase ABC and ACII digestion and reactivity with monoclonal antibodies. Carbohydrate Research, 255:241-254. doi: 10.1016/S0008-6215(00)90982-0
Salanti, A., and Clausen, T. M. et al. 2015. Targeting human cancer by a glycosaminoglycan binding malaria protein. Cancer Cell. 28(4):500-514. doi: 10.1016/j.ccell.2015.09.003
Spliid, C. B. et al. 2021. The specificity of the malarial VAR2CSA protein for chondroitin sulfate depends on 4-O-sulfation and ligand accessibility. J. Biol. Chem. 297(6):101391. doi: 10.1016/j.jbc.2021.101391
Wang, K. et al. 2021. Cryo-EM reveals the architecture of placental malaria VAR2CSA and provides molecular insight into chondroitin sulfate binding. Nat. Commun. 12(1):2956. doi: 10.1038/s41467-021-23254-1

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. An antibody or an antibody variant that binds specifically to a chondroitin sulfate (CS) glycosaminoglycan chain, wherein the antibody or the antibody variant comprises a paratope defined by the following combination of amino acid sequences of LCDR1, LCDR2, LCDR3, HCDR1, HCDR2, and HCDR3, respectively:
SEQ ID NOs: 2, 3, 4, 5, 6, and 7; or
SEQ ID NOs: 9, 10, 11, 12, 13, and 14; or
SEQ ID NOs: 16, 17, 18, 19, 20, and 21; or
SEQ ID NOs: 23, 24, 25, 26, 27, and 28; or
SEQ ID NOs: 30, 31, 32, 33, 34, and 35; or
SEQ ID NOs: 37, 38, 39, 40, 41, and 42; or
SEQ ID NOs: 44, 45, 46, 47, 48, and 49; or
SEQ ID NOs: 52, 53, 54, 55, 56, and 57; or
SEQ ID NOs: 59, 60, 61, 62, 63, and 64; or
SEQ ID NOs: 66, 67, 68, 69, 70, and 71; or
SEQ ID NOs: 73, 74, 75, 76, 77, and 78; or
SEQ ID NOs: 80, 81, 82, 83, 84, and 85; or
SEQ ID NOs: 87, 88, 89, 90, 91, and 92; or
SEQ ID NOs: 94, 95, 96, 97, 98, and 99; or
SEQ ID NOs: 101, 102, 103, 104, 105, and 106; or
SEQ ID NOs: 108, 109, 110, 111, 112, and 113; or
SEQ ID NOs: 115, 116, 117, 118, 119, and 120; or
SEQ ID NOs: 122, 123, 124, 125, 126, and 127; or
SEQ ID NOs: 129, 130, 131, 132, 133, and 134.

66. The antibody or the antibody variant according to claim 65, wherein the antibody or the antibody variant exhibits a higher binding affinity for oncofetal CS (ofCS) than for a non-oncofetal glycosaminoglycans, such as for heparin sulphate and for hyaluronic acid.

67. The antibody or the antibody variant according to claim 65 or 66, which
competes for binding to the CS glycosaminoglycan chain with VAR2CSA (SEQ ID NO: 135) or a CS binding fragment thereof, such as the polypeptide having SEQ ID NO: 136, and/or
competes for binding to the CS glycosaminoglycan with an scFv having any one of SEQ ID NOs: 1, 8, 15, 22, 29, 36, 43, 50, 51, 58, 65, 72, 79, 86, 93, 100, 107, 114, 121, and 128.

68. The antibody or the antibody variant according to claim 1, wherein the binding site is characterized by the presence of a positively charged groove along the V_{H}/V_{L} boundary.

69. The antibody or the antibody variant according to claim 68, wherein the positively charged groove is constituted by positively charged and surface exposed amino acid residues present in the variable loops of both the V_{L} and V_{H} domain.

70. The antibody or the antibody variant according to claim 69, wherein the number of positively charged amino acid residues is 3, 4, 5, or 6.

71. The antibody or the antibody variant according to claim 69 or 70, wherein the distance between the α carbon atoms of the positively charged amino acids across the V_{H}/V_{L} boundary is at most 12 Å, such as in the range 6-12 Å, preferably between 6.7 and 11.4 Å.

72. The antibody or the antibody variant according to claim 65, which exhibits a higher affinity for ofCS than antibodies 2H6, CS56, BE-123, and PG-4.

73. The antibody or the antibody variant according to claim 65, which is different from an IgM antibody.

74. The antibody or the antibody variant according to claim 65, which binds to native ofCS.

75. The antibody or the antibody variant according to claim 65, which is, or is derived from, an IgG antibody.

76. The antibody or the antibody variant according to claim 65, which is, or is derived from, a human antibody.

77. The antibody or the antibody variant according to claim 65, wherein the antibody or the antibody variant competes for binding to ofCS with a second antibody, wherein the second antibody comprises a heavy chain variable region (V_{H}) with an amino acid sequence present in any one of SEQ ID NOs: 1, 8, 15, 22, 29, 36, 43, 50, 51, 58, 65, 72, 79, 86, 93, 100, 107, 114, 121, and 128, and comprises a light chain variable region (V_{L}) with an amino acid sequence present in any one of SEQ ID NOs: 1, 8, 15, 22, 29, 36, 43, 50, 51, 58, 65, 72, 79, 86, 93, 100, 107, 114, 121, and 128.

78. The antibody or the antibody variant according to claim 77, wherein the V_{H} region of the second antibody has
an amino acid sequence present in or identical to the amino acid stretch which is linked C-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, or
an amino acid sequence present in or identical to the amino acid stretch which is linked C-terminally to the sequence GGGGSGGGGSGGGGSGGG (SEQ ID NO: 138) in SEQ ID NO: 121.

79. The antibody or the antibody variant according to claim 77, wherein the V_{L} region of the second antibody has
an amino acid sequence present in or identical to the amino acid stretch which is linked N-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, or
an amino acid sequence present in or identical to the amino acid stretch which is linked N-terminally to the sequence GGGGSGGGGSGGGGSGGG (SEQ ID NO: 138) in SEQ ID NO: 121.

80. The antibody or the antibody variant according to claim 65, wherein the antibody or the antibody variant comprises a combination of
a V_{H} region having an amino acid sequence present in or identical to the amino acid stretch which is linked C-terminally to the sequence GGGGSGGGGSGGGSGGGGS (SEQ ID NO: 137) in any one of SEQ ID NOs: 1, 8, 15, 22, 29, 36, 43, 50, 51, 58, 65, 72, 79, 86, 93, 100, 107, 114, and 128, and
a V_{L} region having an amino acid sequence present in or identical to the amino acid stretch which is linked N-terminally to the sequence GGGGSGGGGSGGGSGGGGS (SEQ ID NO: 137) in the same SEQ ID NO,
or
a V_{H} region having an amino acid sequence present in or identical to the amino acid stretch which is linked C-terminally to the sequence GGGGSGGGGSGGGGSGGG (SEQ ID NO: 138) in SEQ ID NO: 121, and
a V_{L} region having an amino acid sequence present in or identical to the amino acid stretch which is linked N-terminally to the sequence GGGGSGGGGSGGGGSGGG (SEQ ID NO: 138) in the same SEQ ID NO,
wherein each of the V_{H} and V_{L} sequences independently can also be a sequence having at least 80% sequence similarity with the SEQ ID NO indicated.

81. The antibody or the antibody variant according to claim 80, wherein the sequence identity is selected from at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, and at least 99%.

82. The antibody or the antibody variant according to claim 65, wherein the CS glycosaminoglycan chain has N-acetylgalactosamine (GalNAc) residues with a sulfate group at the C-4 position in >50%, such as >60%, preferably >70%, of the disaccharide repeats of the chain.

83. The antibody or the antibody variant according to claim 82, wherein the CS chain contains 40 sulfated GalNac and iduronic acid (IdoA).

84. The antibody or the antibody variant according to any one of the preceding claims, wherein the antibody or the antibody variant binds to the ofCS glycosaminoglycan chain with an equilibrium dissociation constant (K_{D}) of <10 nM, such as <5 nM.

85. The antibody or the antibody variant according to any one of the preceding claims, wherein the ofCS glycosaminoglycan chain is attached to a protein core forming a chondroitin sulfate proteoglycan (CSPG) present in secreted form, on a cell membrane or in an extracellular matrix.

86. The antibody or the antibody variant according to claim 85, wherein the CSPG is selected from any one of: Brain natriuretic peptide B, Endothelial cell-specific molecule 1, Sushi repeat-containing protein SRPX, Decorin, Protein AMBP, Biglycan, Bone marrow proteoglycan, Syndecan-4, Amyloid-like protein 2, HLA class II histocompatibility antigen gamma chain, Chondroitin sulfate proteoglycan 4, Agrin, Testican 1-3, Neuropilin, CD44 antigen, Glypican-1-6, Syndecan-1-34, Laminin subunit gamma 2, Carbonic anhydrase 9, Aggrecan, Perlecan, Collagen alpha-1 (XII), Collagen alpha-1 (XV), Collagen alpha-1 (XVIII), Laminin subunit alpha-4, Matrix-remodeling associated protein 5, Nidogen-2, and Versican.

87. The antibody or the antibody variant according to claim 65, wherein the antibody variant is selected from an Fab, an Fab′, an Fab-SH, an F(ab)2, an F(ab′)₂, an ScFv, an Fv fragment, a Heavy chain Ig (such as a llama or camel Ig), an IgY, a V_{HH} fragment, a dsFV, a minibody, a diabody, a triabody, a kappa body, an IgNAR, a tandAb, a BiTE, and a multispecific antibody.

88. The antibody or the antibody variant according to claim 65, wherein the antibody variant is a bispecific antibody.

89. The antibody or the antibody variant according to claim 88, wherein the bispecific antibody binds specifically to CD3.

90. A conjugate or a fusion protein comprising at least a first and a second moiety, wherein the first moiety is the antibody or the antibody variant according to any one of the preceding claims, and the second moiety is a molecule or polypeptide; wherein the first moiety is conjugated or genetically fused to the second moiety, and wherein the second moiety provides or improves the therapeutic and/or diagnostic function of the conjugate or the fusion protein.

91. The conjugate or the fusion protein according to claim 90, wherein the second moiety is selected from any one of: a toxin or a fragment thereof, an immune-modulating molecule or a fragment thereof, a nanoparticle, a radionuclide or a radionuclide-containing substance, and a label.

92. The conjugate or the fusion protein according to claim 91, wherein the second moiety is a toxin.

93. The conjugate according to claim 92, which toxin is selected from a cytotoxic or cytostatic agent, such as an alkylating agent, an antimetabolite, an anti-microtubule agent such as monomethyl auristatin E (MMAE), a topoisomerase inhibitor such as exetechan, and a cytotoxic antibiotic.

94. The conjugate or the fusion peptide according to claim 90, wherein the second moiety is a polypeptide, and wherein the first moiety is genetically fused to the second moiety.

95. The conjugate according to claim 94, wherein the second moiety is a cytokine or a chemokine, such as interleukin-2 (IL-2), interleukin-7 (IL-7), interleukin-12, granulocyte-macrophage colony-stimulating factor (GM-CSF), tumour necrosis factor (TNF), or TNF-related apoptosis-inducing ligand (TRAIL).

96. A polypeptide comprising
i. a first polypeptide domain being a transmembrane domain and an endodomain of a chimeric antigen receptor (CAR), and
ii. a second polypeptide domain being the antibody or the antibody variant according to claim 65.

97. A CAR-T cell comprising the polypeptide according to claim 96.

98. An isolated nucleic acid molecule comprising a nucleotide sequence encoding the antibody or the antibody variant according to claim 65, the conjugate or the fusion protein according to claim 90, or the polypeptide according to claim 96.

99. A vector comprising the isolated nucleic acid molecule according to claim 98, such as an expression vector or a cloning vector.

100. A host cell comprising or transformed with the vector according to claim 99.

101. A method for producing the antibody or the antibody variant according to claim 65, the conjugate or the fusion protein according to claim 90, or the polypeptide according to claim 96, the method comprising the steps of:
transfecting or transforming a host cell with the vector according to claim 99,
expressing the nucleotide sequence according to claim 98, and
isolating the antibody or the antibody variant, the conjugate or the fusion protein, or the polypeptide.

102. A pharmaceutical composition comprising the antibody or the antibody variant according to claim 65, the conjugate or the fusion protein according to claim 90, or the CAR-T cell according to claim 97, and a pharmaceutically acceptable carrier, vehicle or diluent.

103. Use of the antibody or the antibody variant according to claim 65, or the conjugate or the fusion protein according to claim 90, for in vitro detection and/or isolation of cancer cells, such as cancer cells derived from a subject, such as circulating tumour cells.

104. The use according to claim 103, wherein the cancer cells are derived from a cancer selected from an epithelial tumour, a non-epithelial tumour, and a mixed tumour.

105. The use according to claim 104, wherein the epithelial tumour is a carcinoma or an adenocarcinoma, and wherein the non-epithelial tumour or mixed tumour is a liposarcoma, a fibrosarcoma, a chondrosarcoma, an osteosarcoma, a leiomyosarcoma, a rhabomyosarcoma, a glioma, a neuroblastoma, a medullablastoma, a malignant melanoma, a malignant meningioma, a neurofibrosarcoma, a leukemia, a myeloproleferative disorder, a lymphoma (such as a B-cell or T-cell lymphoma), a hemangiosarcoma, a Kaposi's sarcoma, a malignant teratoma, a dysgerminoma, a seminoma, or a choriosarcomamelanoma.

106. The use according to claim 104 or 105, wherein the cancer is a cancer of the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the ovaries, the uterus, the uterine cervix, the spleen, bone, or cartilage.

107. The antibody or the antibody variant according to claim 65, the conjugate or the fusion protein according claim 90, the CAR-T cell according to claim 97 or the pharmaceutical composition according to claim 102, for use as a medicament.

108. The antibody or the antibody variant according to claim 65, or the conjugate or the fusion protein according to claim 90, or the pharmaceutical composition according to claim 102, for use in diagnosing cancer in a subject.

109. The antibody or the antibody variant according to claim 65, the conjugate or the fusion protein according to claim 90, the CAR-T cell according to claim 97, or the pharmaceutical composition according to claim 102, for use in treating cancer in a subject in need thereof, or for use in the treatment of a condition involving expression, such as inappropriate expression, of ofCS, such as in a condition selected from the group consisting of arthritis, arthrosis, multiple sclerosis, healing after neural damage, cartilage repair, wound healing, and psoriasis, in a subject in need thereof, or for use in delivering a therapeutic to the placenta in a subject in need thereof.

110. The antibody or the antibody variant for the use according to claim 108 or 109, wherein the subject is a mammal, such as a canine, feline, equine, or a human.

111. The antibody or the antibody variant, the conjugate or the fusion protein, the CAR-T cell or the pharmaceutical composition, for use according to claim 108, wherein the cancer is as defined in any one of claims 104-106.

112. A method of treating a cancer or for treating a condition involving expression, such as inappropriate expression, of ofCS, such as in a condition selected from the group consisting of arthritis, arthrosis, multiple sclerosis, healing after neural damage, cartilage repair, wound healing, and psoriasis, or for use in delivering a therapeutic to the placenta, in a subject in need thereof, the method comprising administering to the subject a therapeutically effective amount of the antibody or the antibody variant according to claim 65, the conjugate or the fusion protein according to claim 90, the CAR-T cell according to claim 97, or the pharmaceutical composition according to claim 102.

113. The method according to claim 112, wherein the cancer is wherein the cancer is as defined in any one of claims 104-106, and/or wherein the subject is as defined in claim 110.

114. A method for detection of ofCS in a sample, the method comprising
1) contacting said sample with an antibody or antibody variant according to any one of claim 65 to allow the antibody or the antibody variant to specifically bind ofCS, subsequently contacting the antibody or antibody variant and any ofCS bound thereby with an agent, which is capable of binding ofCS, to allow said agent to bind ofCS, or
2) contacting said sample with an agent, which is capable of binding ofCS, to allow the agent to bind ofCS, subsequently contacting the agent and any ofCS bound thereby with an antibody or antibody variant according to any one of claim 65 to allow the antibody to bind ofCS,
and detecting quantitatively or qualitatively the presence of complexes comprising the ofCS, the antibody or antibody variant, and the agent,
wherein the agent and the antibody or antibody variant are non-competitive for their binding to ofCS.

115. The method according to claim 114, wherein the sample is a sample comprising or consisting of cells and/or tissue, preferably a bodily fluid, such as blood, urine, saliva, CS fluid, and lymph; feces; and a biopsy.

116. The method according to claim 114, option 1, wherein the agent is an antibody or antibody fragment according to claim 65, VAR2CSA (SEQ ID NO: 135) or a CSA binding fragment thereof such as a protein consisting of or comprising SEQ ID NO: 136, an antibody that binds chondroitin sulfate, an antibody that binds the protein core of a proteoglycan, or a glycosaminoglycan staining dye.

117. The method according to claim 114, option 2, wherein the agent is VAR2CSA (SEQ ID NO: 135) or a CSA binding fragment thereof or an antibody that binds the protein core of a proteoglycan.

118. The method according to claim 116, wherein the agent is an antibody capable of binding the protein core of a proteoglycan.

119. The method according to claim 114, wherein the antibody or the agent is coupled to a solid or semi-solid phase.

120. The method according to claim 114, wherein detection of the complex is facilitated by the antibody or the agent being labelled with a detectable moiety or by adding a detectable agent, which binds the antibody or agent.

121. A detection kit comprising an antibody according to claim 65, an agent as defined in claim 114 or 116, and a reaction vessel, and optionally also a labelled agent, which binds the antibody or agent.

122. A method for providing nucleic acids encoding the antibody or the antibody variant according to claim 65 comprising screening a library comprising antigen-binding fragments of antibodies, wherein the library is comprised of display agents, which are selected from cells, virus, and phage comprising antibody-encoding nucleic acid fragments, for binding of the display agents to a capture agent, wherein the capture agent consists essentially of purified ofCS or ofCS-decorated protein, and subsequently isolating antibody coding nucleic fragments from the capture agent-binding display agents, and optionally sequencing the isolated nucleic acid fragments.

123. The method according to claim 122, wherein the display agent is a yeast cell or a phage selected from the phages M3, fd, T4, T7 and λ.

124. The method according to claim 122 or 123, wherein the antigen-binding fragment of antibodies are in the format of scFV, F(ab), F(ab′)₂, and F(ab′).

125. The method according to claim 122, wherein the antigen binding fragments contain synthetic CDRs, semi-synthetic CDRs, or CDRs derived from a human antibody library.

126. A method of producing an antibody or antibody variant according to any one of claim 65, comprising expressing an expression vector comprising a nucleic acid fragment obtained according to the method of claim 122 in a host cell in a culture and subsequently isolating the expression product from the culture.


SEQUENCE LISTING (139 sequences)

SEQ ID NO: 1 - protein, 254 aa, synthetic construct
  REGION 1..254: note = Antibody construct
DIVMTQSPDSLAVSLGERATINCKSSQSVLGSSNNKNYLAWYQQKPGQPPKLLIYWASTRESGVPDRFSGSGSGTDFTLTISSLQAEDVAVYYCQQYYRSPYTFGQGTKVEIKGGGGSGGGGSGGGSGGGGSEVQLLESGGGLVQPGGSLRLSCAASGFTFSDYAMSWVRQAPGKGLEWVSRISGSGGQTKYADSVKGRFTISRDNSKNTLYLQMNSLRAEDTAVYYCARGHSSSWLRGHFQHWGQGTLVTVSS

SEQ ID NO: 2 - protein, 17 aa, synthetic construct
  REGION 1..17: note = Antibody construct
KSSQSVLGSSNNKNYLA

SEQ ID NO: 3 - protein, 7 aa, synthetic construct
  REGION 1..7: note = Antibody construct
WASTRES

SEQ ID NO: 4 - protein, 9 aa, synthetic construct
  REGION 1..9: note = Antibody construct
QQYYRSPYT

SEQ ID NO: 5 - protein, 10 aa, synthetic construct
  REGION 1..10: note = Antibody construct
GFTFSDYAMS

SEQ ID NO: 6 - protein, 17 aa, synthetic construct
  REGION 1..17: note = Antibody construct
RISGSGGQTKYADSVKG

SEQ ID NO: 7 - protein, 13 aa, synthetic construct
  REGION 1..13: note = Antibody construct
GHSSSWLRGHFQH

SEQ ID NO: 8 - protein, 253 aa, synthetic construct
  REGION 1..253: note = Antibody construct
DIVMTQSPDSLAVSLGERATINCKSSQSVLRSGNNKNRLAWYQQKPGQPPKLLIYDASTRESGVPDRFSGSGSGTDFTLTISSLQAEDVAVYYCQQQYNYPYTFGQGTKVEIKGGGGSGGGGSGGGSGGGGSEVQLLESGGGLVQPGGSLRLSCAASGFTFTSSAMSWVRQAPGKGLEWVSRIDGSGGDTRYADSVKGRFTISRDNSKNTLYLQMNSLRAEDTAVYYCAKGRRLWFGDPFDHWGQGTLVTVSS

SEQ ID NO: 9 - protein, 17 aa, synthetic construct
  REGION 1..17: note = Antibody construct
KSSQSVLRSGNNKNRLA

SEQ ID NO: 10 - protein, 7 aa, synthetic construct
  REGION 1..7: note = Antibody construct
DASTRES

SEQ ID NO: 11 - protein, 9 aa, synthetic construct
  REGION 1..9: note = Antibody construct
QQQYNYPYT

SEQ ID NO: 12 - protein, 10 aa, synthetic construct
  REGION 1..10: note = Antibody construct
GFTFTSSAMS

SEQ ID NO: 13 - protein, 17 aa, synthetic construct
  REGION 1..17: note = Antibody construct
RIDGSGGDTRYADSVKG

SEQ ID NO: 14 - protein, 12 aa, synthetic construct
  REGION 1..12: note = Antibody construct
GRRLWFGDPFDH

SEQ ID NO: 15 - protein, 253 aa, synthetic construct
  REGION 1..253: note = Antibody construct
DIVMTQSPDSLAVSLGERATINCKSSQSVLSSSNNKNRLAWYQQKPGQPPKLLIYAASTRESGVPDRFSGSGSGTDFTLTISSLQAEDVAVYYCQQGYSSPWTFGQGTKVEIKGGGGSGGGGSGGGSGGGGSEVQLLESGGGLVQPGGSLRLSCAASGFTFSNYGMSWVRQAPGKGLEWVSRISGSGGYTNYADSVKGRFTISRDNSKNTLYLQMNSLRAEDTAVYYCARVYFDGTTRYFDSWGPGTLVTVSS

SEQ ID NO: 16 - protein, 17 aa, synthetic construct
  REGION 1..17: note = Antibody construct
KSSQSVLSSSNNKNRLA

SEQ ID NO: 17 - protein, 7 aa, synthetic construct
  REGION 1..7: note = Antibody construct
AASTRES

SEQ ID NO: 18 - protein, 9 aa, synthetic construct
  REGION 1..9: note = Antibody construct
QQGYSSPWT

SEQ ID NO: 19 - protein, 10 aa, synthetic construct
  REGION 1..10: note = Antibody construct
GFTFSNYGMS

SEQ ID NO: 20 - protein, 17 aa, synthetic construct
  REGION 1..17: note = Antibody construct
RISGSGGYTNYADSVKG

SEQ ID NO: 21 - protein, 12 aa, synthetic construct
  REGION 1..12: note = Antibody construct
VYFDGTTRYFDS

SEQ ID NO: 22 - protein, 258 aa, synthetic construct
  REGION 1..258: note = Antibody construct
DIVMTQSPDSLAVSLGERATINCKSSQSVLRSGNNKNRLAWYQQKPGQPPKLLIYAASTRESGVPDRFSGSGSGTDFTLTISSLQAEDVAVYYCQQSYRAPITFGQGTKVEIKGGGGSGGGGSGGGSGGGGSEVQLLESGGGLVQPGGSLRLSCAASGFTFTDYAMSWVRQAPGKGLEWVSAIDGGGDSTYYADSVKGRFTISRDNSKNTLYLQMNSLRAEDTAVYYCARAKFGSRRPERGMGYFDLWGRGTLVTVSS

SEQ ID NO: 23 - protein, 17 aa, synthetic construct
  REGION 1..17: note = Antibody construct
KSSQSVLRSGNNKNRLA

SEQ ID NO: 24 - protein, 7 aa, synthetic construct
  REGION 1..7: note = Antibody construct
AASTRES

SEQ ID NO: 25 - protein, 9 aa, synthetic construct
  REGION 1..9: note = Antibody construct
QQSYRAPIT

SEQ ID NO: 26 - protein, 10 aa, synthetic construct
  REGION 1..10: note = Antibody construct
GFTFTDYAMS

SEQ ID NO: 27 - protein, 17 aa, synthetic construct
  REGION 1..17: note = Antibody construct
AIDGGGDSTYYADSVKG

SEQ ID NO: 28 - protein, 17 aa, synthetic construct
  REGION 1..17: note = Antibody construct
AKFGSRRPERGMGYFDL

SEQ ID NO: 29 - protein, 252 aa, synthetic construct
  REGION 1..252: note = Antibody construct
DIVMTQSPDSLAVSLGERATINCKSSQSVLRSSNNKNRLAWYQQKPGQPPKLLIYAASTRESGVPDRFSGSGSGTDFTLTISSLQAEDVAVYYCQQGYRAPFTFGQGTKVEIKGGGGSGGGGSGGGSGGGGSEVQLLESGGGLVQPGGSLRLSCAASGFTFSNYAMSWVRQAPGKGLEWVSRIDGSGGTTNYADSVKGRFTISRDNSKNTLYLQMNSLRAEDTAVYYCAKSRGYNYGYFDYWGQGTLVTVSS

SEQ ID NO: 30 - protein, 17 aa, synthetic construct
  REGION 1..17: note = Antibody construct
KSSQSVLRSSNNKNRLA

SEQ ID NO: 31 - protein, 7 aa, synthetic construct
  REGION 1..7: note = Antibody construct
AASTRES

SEQ ID NO: 32 - protein, 9 aa, synthetic construct
  REGION 1..9: note = Antibody construct
QQGYRAPFT

SEQ ID NO: 33 - protein, 10 aa, synthetic construct
  REGION 1..10: note = Antibody construct
GFTFSNYAMS

SEQ ID NO: 34 - protein, 17 aa, synthetic construct
  REGION 1..17: note = Antibody construct
RIDGSGGTTNYADSVKG

SEQ ID NO: 35 - protein, 11 aa, synthetic construct
  REGION 1..11: note = Antibody construct
SRGYNYGYFDY

SEQ ID NO: 36 - protein, 251 aa, synthetic construct
  REGION 1..251: note = Antibody construct
DIVMTQSPDSLAVSLGERATINCKSSQSVLRSSNNKNRLAWYQQKPGQPPKLLIYAASTRESGVPDRFSGSGSGTDFTLTISSLQAEDVAVYYCQQGYNAPLTFGQGTKVEIKGGGGSGGGGSGGGSGGGGSEVQLLESGGGLVQPGGSLRLSCAASGFTFTNYAMSWVRQAPGKGLEWVSRISGSGSSTSYADSVKGRFTISRDNSKNTLYLQMNSLRAEDTAVYYCARFRLRSLYFDYWGQGTLVTVSS

SEQ ID NO: 37 - protein, 17 aa, synthetic construct
  REGION 1..17: note = Antibody construct
KSSQSVLRSSNNKNRLA

SEQ ID NO: 38 - protein, 7 aa, synthetic construct
  REGION 1..7: note = Antibody construct
AASTRES

SEQ ID NO: 39 - protein, 9 aa, synthetic construct
  REGION 1..9: note = Antibody construct
QQGYNAPLT

SEQ ID NO: 40 - protein, 10 aa, synthetic construct
  REGION 1..10: note = Antibody construct
GFTFTNYAMS

SEQ ID NO: 41 - protein, 17 aa, synthetic construct
  REGION 1..17: note = Antibody construct
RISGSGSSTSYADSVKG

SEQ ID NO: 42 - protein, 10 aa, synthetic construct
  REGION 1..10: note = Antibody construct
FRLRSLYFDY

SEQ ID NO: 43 - protein, 252 aa, synthetic construct
  REGION 1..252: note = Antibody construct
DIVMTQSPDSLAVSLGERATINCKSSQSVLRSSNNKNRLAWYQQKPGQPPKLLIYGASTRESGVPDRFSGSGSGTDFTLTISSLQAEDVAVYYCQQGYNSPLTFGQGTKVEIKGGGGSGGGGSGGGSGGGGSEVQLLESGGGLVQPGGSLRLSCAASGFTFTNYAMSWVRQAPGKGLEWVSWISGRGGSTNYADSVKGRFTISRDNSKNTLYLQMNSLRAEDTAVYYCARARARSSGYFDYWGQGTLVTVSS

SEQ ID NO: 44 - protein, 17 aa, synthetic construct
  REGION 1..17: note = Antibody construct
KSSQSVLRSSNNKNRLA

SEQ ID NO: 45 - protein, 7 aa, synthetic construct
  REGION 1..7: note = Antibody construct
GASTRES

SEQ ID NO: 46 - protein, 9 aa, synthetic construct
  REGION 1..9: note = Antibody construct
QQGYNSPLT

SEQ ID NO: 47 - protein, 10 aa, synthetic construct
  REGION 1..10: note = Antibody construct
GFTFTNYAMS

SEQ ID NO: 48 - protein, 17 aa, synthetic construct
  REGION 1..17: note = Antibody construct
WISGRGGSTNYADSVKG

SEQ ID NO: 49 - protein, 11 aa, synthetic construct
  REGION 1..11: note = Antibody construct
ARARSSGYFDY

SEQ ID NO: 50 - protein, 239 aa, synthetic construct
  REGION 1..239: note = Antibody construct
DIVMTQSPDSLAVSLGERATINCKSSQSVLRSGNNKNRLAWYQQKPGQPPKLLIYGASTRESGVPDRFSGSGSGTDFTLTISSLQAEDVAVYYCQQGYSSPITFGQGTKVEIKGGGGSEVQLLESGGGLVQPGGSLRLSCAASGFTFSDYYMSWVRQAPGKGLEWVSRIDGGGGSTYYADSVKGRFTISRDNSKNTLYLQMNSLRAEDTAVYYCAKVRYPTGPYYFDYWGQGTLVTVSS

SEQ ID NO: 51 - protein, 253 aa, synthetic construct
  REGION 1..253: note = Antibody construct
DIVMTQSPDSLAVSLGERATINCKSSQSVLRSGNNKNRLAWYQQKPGQPPKLLIYGASTRESGVPDRFSGSGSGTDFTLTISSLQAEDVAVYYCQQGYSSPITFGQGTKVEIKGGGGSGGGGSGGGSGGGGSEVQLLESGGGLVQPGGSLRLSCAASGFTFSDYYMSWVRQAPGKGLEWVSRIDGGGGSTYYADSVKGRFTISRDNSKNTLYLQMNSLRAEDTAVYYCAKVRYPTGPYYFDYWGQGTLVTVSS

SEQ ID NO: 52 - protein, 17 aa, synthetic construct
  REGION 1..17: note = Antibody construct
KSSQSVLRSGNNKNRLA

SEQ ID NO: 53 - protein, 7 aa, synthetic construct
  REGION 1..7: note = Antibody construct
GASTRES

SEQ ID NO: 54 - protein, 9 aa, synthetic construct
  REGION 1..9: note = Antibody construct
QQGYSSPIT

SEQ ID NO: 55 - protein, 10 aa, synthetic construct
  REGION 1..10: note = Antibody construct
GFTFSDYYMS

SEQ ID NO: 56 - protein, 17 aa, synthetic construct
  REGION 1..17: note = Antibody construct
RIDGGGGSTYYADSVKG

SEQ ID NO: 57 - protein, 12 aa, synthetic construct
  REGION 1..12: note = Antibody construct
VRYPTGPYYFDY

SEQ ID NO: 58 - protein, 254 aa, synthetic construct
  REGION 1..254: note = Antibody construct
DIVMTQSPDSLAVSLGERATINCKSSQSVLRSNNNKNRLAWYQQKPGQPPKLLIYGASTRESGVPDRFSGSGSGTDFTLTISSLQAEDVAVYYCQQGYSYPLTFGQGTKVEIKGGGGSGGGGSGGGSGGGGSEVQLFESGGGLVQPGGSLRLSCAASGFTFTDYAMSWVRQAPGKGLEWVSAIQGGGGGTRYADSVKGRFTISRDNSKNTLYLQMNSLRAEDTAVYYCARDRRPTVTTSYFDYWGQGTLVTVSS

SEQ ID NO: 59 - protein, 17 aa, synthetic construct
  REGION 1..17: note = Antibody construct
KSSQSVLRSNNNKNRLA

SEQ ID NO: 60 - protein, 7 aa, synthetic construct
  REGION 1..7: note = Antibody construct
GASTRES

SEQ ID NO: 61 - protein, 9 aa, synthetic construct
  REGION 1..9: note = Antibody construct
QQGYSYPLT

SEQ ID NO: 62 - protein, 10 aa, synthetic construct
  REGION 1..10: note = Antibody construct
GFTFTDYAMS

SEQ ID NO: 63 - protein, 17 aa, synthetic construct
  REGION 1..17: note = Antibody construct
AIQGGGGGTRYADSVKG

SEQ ID NO: 64 - protein, 13 aa, synthetic construct
  REGION 1..13: note = Antibody construct
DRRPTVTTSYFDY

SEQ ID NO: 65 - protein, 248 aa, synthetic construct
  REGION 1..248: note = Antibody construct
DIVMTQSPDSLAVSLGERATINCKSSQSVLRSGNNKNRLAWYQQKPGQPPKLLIYWASTRESGVPDRFSGSGSGTDFTLTISSLQAEDVAVYYCQQSYNRPLTFGQGTKVEIKGGGGSGGGGSGGGSGGGGSEVQLLESGGGLVQPGGSLRLSCAASGFTFSSYQMSWVRQAPGKGLEWVSEISGYGGSTKYADSVKGRFTISRDNSKNTLYLQMNSLRAEDTAVYYCARRNAAFDIWGQGTMVTVSS

SEQ ID NO: 66 - protein, 17 aa, synthetic construct
  REGION 1..17: note = Antibody construct
KSSQSVLRSGNNKNRLA

SEQ ID NO: 67 - protein, 7 aa, synthetic construct
  REGION 1..7: note = Antibody construct
WASTRES

SEQ ID NO: 68 - protein, 9 aa, synthetic construct
  REGION 1..9: note = Antibody construct
QQSYNRPLT

SEQ ID NO: 69 - protein, 10 aa, synthetic construct
  REGION 1..10: note = Antibody construct
GFTFSSYQMS

SEQ ID NO: 70 - protein, 17 aa, synthetic construct
  REGION 1..17: note = Antibody construct
EISGYGGSTKYADSVKG

SEQ ID NO: 71 - protein, 7 aa, synthetic construct
  REGION 1..7: note = Antibody construct
RNAAFDI

SEQ ID NO: 72 - protein, 255 aa, synthetic construct
  REGION 1..255: note = Antibody construct
DIVMTQSPDSLAVSLGERATINCKSSQSVLRSDNNENRLAWYQQKPGQPPKLLIYAASTRESGVPDRFSGSGSGTDFTLTISSLQAEDVAVYYCQQYYREPITFGQGTKVEIKGGGGSGGGGSGGGSGGGGSEVQLLESGGGLVQPGGSLRLSCAASGFTFTSYGMSWVRQAPGKGLEWVSGISGRGGSTSYADSVKGRFTISRDNSKNTLYLQMNSLRAEDTAVYYCARTRGRIAARRGWFDPWGQGTLVTVSS

SEQ ID NO: 73 - protein, 17 aa, synthetic construct
  REGION 1..17: note = Antibody construct
KSSQSVLRSDNNENRLA

SEQ ID NO: 74 - protein, 7 aa, synthetic construct
  REGION 1..7: note = Antibody construct
AASTRES

SEQ ID NO: 75 - protein, 9 aa, synthetic construct
  REGION 1..9: note = Antibody construct
QQYYREPIT

SEQ ID NO: 76 - protein, 10 aa, synthetic construct
  REGION 1..10: note = Antibody construct
GFTFTSYGMS

SEQ ID NO: 77 - protein, 17 aa, synthetic construct
  REGION 1..17: note = Antibody construct
GISGRGGSTSYADSVKG

SEQ ID NO: 78 - protein, 14 aa, synthetic construct
  REGION 1..14: note = Antibody construct
TRGRIAARRGWFDP

SEQ ID NO: 79 - protein, 256 aa, synthetic construct
  REGION 1..256: note = Antibody construct
DIVMTQSPDSLAVSLGERATINCKSSQSVLRSDNNKNRLAWYQQKPGQPPKLLIYGASTRESGVPDRFSGSGSGTDFTLTISSLQAEDVAVYYCQQGYSSPWTFGQGTKVEIKGGGGSGGGGSGGGSGGGGSEVQLLESGGGLVQPGGSLRLSCAASGFTFTSTAMSWVRQAPGKGLEWVSAISGRGGSTKYADSVKGRFTISRDNSKNTLYLQMNSLRAEDTAVYYCARASKKSPIAAAGTFDLWGQGTLVTVSS

SEQ ID NO: 80 - protein, 17 aa, synthetic construct
  REGION 1..17: note = Antibody construct
KSSQSVLRSDNNKNRLA

SEQ ID NO: 81 - protein, 7 aa, synthetic construct
  REGION 1..7: note = Antibody construct
GASTRES

SEQ ID NO: 82 - protein, 9 aa, synthetic construct
  REGION 1..9: note = Antibody construct
QQGYSSPWT

SEQ ID NO: 83 - protein, 10 aa, synthetic construct
  REGION 1..10: note = Antibody construct
GFTFTSTAMS

SEQ ID NO: 84 - protein, 17 aa, synthetic construct
  REGION 1..17: note = Antibody construct
AISGRGGSTKYADSVKG

SEQ ID NO: 85 - protein, 15 aa, synthetic construct
  REGION 1..15: note = Antibody construct
ASKKSPIAAAGTFDL

SEQ ID NO: 86 - protein, 252 aa, synthetic construct
  REGION 1..252: note = Antibody construct
DIVMTQSPDSLAVSLGERATINCKSSQSVLRSGNNKNRLAWYQQKPGQPPKLLIYGASTRESGVPDRFSGSGSGTDFTLTISSLQAEDVAVYYCQQYYNRPYTFGQGTKVEIKGGGGSGGGGSGGGSGGGGSEVQLLESGGGLVQPGGSLRLSCAASGFTFTDYGMSWVRQAPGKGLEWVSGISGSGGSTYYADSVKGRFTISRDNSKNTLYLQMNSLRAEDTAVYYCAKSRRRDYNWFDPWGQGTLVTVSS

SEQ ID NO: 87 - protein, 17 aa, synthetic construct
  REGION 1..17: note = Antibody construct
KSSQSVLRSGNNKNRLA

SEQ ID NO: 88 - protein, 7 aa, synthetic construct
  REGION 1..7: note = Antibody construct
GASTRES

SEQ ID NO: 89 - protein, 9 aa, synthetic construct
  REGION 1..9: note = Antibody construct
QQYYNRPYT

SEQ ID NO: 90 - protein, 10 aa, synthetic construct
  REGION 1..10: note = Antibody construct
GFTFTDYGMS

SEQ ID NO: 91 - protein, 17 aa, synthetic construct
  REGION 1..17: note = Antibody construct
GISGSGGSTYYADSVKG

SEQ ID NO: 92 - protein, 11 aa, synthetic construct
  REGION 1..11: note = Antibody construct
SRRRDYNWFDP

SEQ ID NO: 93 - protein, 254 aa, synthetic construct
  REGION 1..254: note = Antibody construct
DIVMTQSPDSLAVSLGERATINCKSSQSVLRSGNNKNRLAWYQQKPGQPPKLLIYAASTRESGVPDRFSGSGSGTDFTLTISSLQAEDVAVYYCQQGYREPLTFGQGTKVEIKGGGGSGGGGSGGGSGGGGSEVQLLESGGGLVQPGGSLRLSCAASGFTFSNYYMSWVRQAPGKGLEWVSAISGSGGQTSYADSVKGRFTISRDNSKNTLYLQMNSLRAEDTAVYYCARHRRGRGAMGYFDYWGQGTLVTVSS

SEQ ID NO: 94 - protein, 17 aa, synthetic construct
  REGION 1..17: note = Antibody construct
KSSQSVLRSGNNKNRLA

SEQ ID NO: 95 - protein, 7 aa, synthetic construct
  REGION 1..7: note = Antibody construct
AASTRES

SEQ ID NO: 96 - protein, 9 aa, synthetic construct
  REGION 1..9: note = Antibody construct
QQGYREPLT

SEQ ID NO: 97 - protein, 10 aa, synthetic construct
  REGION 1..10: note = Antibody construct
GFTFSNYYMS

SEQ ID NO: 98 - protein, 17 aa, synthetic construct
  REGION 1..17: note = Antibody construct
AISGSGGQTSYADSVKG

SEQ ID NO: 99 - protein, 13 aa, synthetic construct
  REGION 1..13: note = Antibody construct
HRRGRGAMGYFDY

SEQ ID NO: 100 - protein, 252 aa, synthetic construct
  REGION 1..252: note = Antibody construct
DIVMTQSPDSLAVSLGERATINCKSSQSVLRSGNNKNRLAWYQQKPGQPPKLLIYAASTRESGVPDRFSGSGSGTDFTLTISSLQAEDVAVYYCQQGYSYPYTFGQGTKVEIKGGGGSGGGGSGGGSGGGGSEVQLLESGGGLVQPGGSLRLSCAASGFTFSSYGMSWVRQAPGKGLEWVSDIDGSGGSTYYADSVKGRFTISRDNSKNTLYLQMNSLRAEDTAVYYCARDRRARSHYFDYWGQGTLVTVSS

SEQ ID NO: 101 - protein, 17 aa, synthetic construct
  REGION 1..17: note = Antibody construct
KSSQSVLRSGNNKNRLA

SEQ ID NO: 102 - protein, 7 aa, synthetic construct
  REGION 1..7: note = Antibody construct
AASTRES

SEQ ID NO: 103 - protein, 9 aa, synthetic construct
  REGION 1..9: note = Antibody construct
QQGYSYPYT

SEQ ID NO: 104 - protein, 10 aa, synthetic construct
  REGION 1..10: note = Antibody construct
GFTFSSYGMS

SEQ ID NO: 105 - protein, 17 aa, synthetic construct
  REGION 1..17: note = Antibody construct
DIDGSGGSTYYADSVKG

SEQ ID NO: 106 - protein, 11 aa, synthetic construct
  REGION 1..11: note = Antibody construct
DRRARSHYFDY

SEQ ID NO: 107 - protein, 252 aa, synthetic construct
  REGION 1..252: note = Antibody construct
DIVMTQSPDSLAVSLGERATINCKSSQSVLSSYNNKNRLAWYQQKPGQPPKLLIYAASTRESGVPDRFSGSGSGTDFTLTISSLQAEDVAVYYCQQGYSRPITFGQGTKVEIKGGGGSGGGGSGGGSGGGGSEVQLLESGGGLVQPGGSLRLSCAASGFTFTSYQMSWVRQAPGKGLEWVSVIDGSGGYTRYADSVKGRFTISRDNSKNTLYLQMNSLRAEDTAVYYCASPRRGKGSYFDYWGQGTLVTVSS

SEQ ID NO: 108 - protein, 17 aa, synthetic construct
  REGION 1..17: note = Antibody construct
KSSQSVLSSYNNKNRLA

SEQ ID NO: 109 - protein, 7 aa, synthetic construct
  REGION 1..7: note = Antibody construct
AASTRES

SEQ ID NO: 110 - protein, 9 aa, synthetic construct
  REGION 1..9: note = Antibody construct
QQGYSRPIT

SEQ ID NO: 111 - protein, 10 aa, synthetic construct
  REGION 1..10: note = Antibody construct
GFTFTSYQMS

SEQ ID NO: 112 - protein, 17 aa, synthetic construct
  REGION 1..17: note = Antibody construct
VIDGSGGYTRYADSVKG

SEQ ID NO: 113 - protein, 11 aa, synthetic construct
  REGION 1..11: note = Antibody construct
PRRGKGSYFDY

SEQ ID NO: 114 - protein, 252 aa, synthetic construct
  REGION 1..252: note = Antibody construct
DIVMTQSPDSLAVSLGERATINCKSSQSVLRSGNNKNRLAWYQQKPGQPPKLLIYGASTRESGVPDRFSGSGSGTDFTLTISSLQAEDVAVYYCQQGYSAPITFGQGTKVEIKGGGGSGGGGSGGGSGGGGSEVQLLESGGGLVQPGGSLRLSCAASGFTFSSYAMSWVRQAPGKGLEWVSAISGSGGSTRYADSVKGRFTISRDNSKNTLYLQMNSLRAEDTAVYYCARARSSSGGYFDYWGQGTLVTVSS

SEQ ID NO: 115 - protein, 17 aa, synthetic construct
  REGION 1..17: note = Antibody construct
KSSQSVLRSGNNKNRLA

SEQ ID NO: 116 - protein, 7 aa, synthetic construct
  REGION 1..7: note = Antibody construct
GASTRES

SEQ ID NO: 117 - protein, 9 aa, synthetic construct
  REGION 1..9: note = Antibody construct
QQGYSAPIT

SEQ ID NO: 118 - protein, 10 aa, synthetic construct
  REGION 1..10: note = Antibody construct
GFTFSSYAMS

SEQ ID NO: 119 - protein, 17 aa, synthetic construct
  REGION 1..17: note = Antibody construct
AISGSGGSTRYADSVKG

SEQ ID NO: 120 - protein, 11 aa, synthetic construct
  REGION 1..11: note = Antibody construct
ARSSSGGYFDY

SEQ ID NO: 121 - protein, 244 aa, synthetic construct
  REGION 1..244: note = Antibody construct
QPVLTQPPSASGTPGQRVTISCSGSSSNIGNNPVNWYQQLPGTAPKLLIYRNNQRPSGVPDRFSGSKSGTSASLAISGLRSEDEAHYYCAAWNDRLSGPRVFGGGTKLTVLGGGGSGGGGSGGGGSGGGQVQLVQSGGGLAQPGGSLRLSCAASGFTFRSYWMTWARQAPGKGLEWVATIKQDGSEKYYVDSVKGRFTISRDDATNSLFLQMNSLRAEDTALYYCARARNMDVWGQGTTVTVSS

SEQ ID NO: 122 - protein, 13 aa, synthetic construct
  REGION 1..13: note = Antibody construct
SGSSSNIGNNPVN

SEQ ID NO: 123 - protein, 7 aa, synthetic construct
  REGION 1..7: note = Antibody construct
RNNQRPS

SEQ ID NO: 124 - protein, 12 aa, synthetic construct
  REGION 1..12: note = Antibody construct
AAWNDRLSGPRV

SEQ ID NO: 125 - protein, 10 aa, synthetic construct
  REGION 1..10: note = Antibody construct
GFTFRSYWMT

SEQ ID NO: 126 - protein, 17 aa, synthetic construct
  REGION 1..17: note = Antibody construct
TIKQDGSEKYYVDSVKG

SEQ ID NO: 127 - protein, 6 aa, synthetic construct
  REGION 1..6: note = Antibody construct
ARNMDV

SEQ ID NO: 128 - protein, 247 aa, synthetic construct
  REGION 1..247: note = Antibody construct
DIVMTQSPSSMSASVGDRVTITCRASQGLSNRLAWYQQKPGKAPKLLIYAASSLQSGVPSRFSGSGSGTDFTLTISRLQPEDFATYYCQQANSFPLTFGQGTRLEIKGGGGSGGGGSGGGSGGGGSQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGGSSGWRKYFQHWGQGTLVTVSS

SEQ ID NO: 129 - protein, 11 aa, synthetic construct
  REGION 1..11: note = Antibody construct
RASQGLSNRLA

SEQ ID NO: 130 - protein, 7 aa, synthetic construct
  REGION 1..7: note = Antibody construct
AASSLQS

SEQ ID NO: 131 - protein, 9 aa, synthetic construct
  REGION 1..9: note = Antibody construct
QQANSFPLT

SEQ ID NO: 132 - protein, 10 aa, synthetic construct
  REGION 1..10: note = Antibody construct
GYTFTGYYMH

SEQ ID NO: 133 - protein, 17 aa, synthetic construct
  REGION 1..17: note = Antibody construct
WINPNSGGTNYAQKFQG

SEQ ID NO: 134 - protein, 12 aa, synthetic construct
  REGION 1..12: note = Antibody construct
GGSSGWRKYFQH

SEQ ID NO: 135 - protein, 2700 aa, synthetic construct
  REGION 1..2700: note = VAR2CSA construct
MDSTSTIANKIEEYLGAKSDDSKIDELLKADPSEVEYYRSGGDGDYLKNNICKITVNHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFNEKLKNAEKKCKENESTDTNINKSETSCDLNATNYIRGCQSKTYDGKIFPGKGGEKQWICKDTIIHGDTNGACIPPRTQNLCVGELWDKSYGGRSNIKNDTKELLKEKIKNAIHKETELLYEYHDTGTAIISKNDKKGQKGKNDPNGLPKGFCHAVQRSFIDYKNMILGTSVNIYEHIGKLQEDIKKIIEKGTPQQKDKIGGVGSSTENVNAWWKGIEREMWDAVRCAITKINKKNNNSIFNGDECGVSPPTGNDEDQSVSWFKEWGEQFCIERLRYEQNIREACTINGKNEKKCINSKSGQGDKIQGACKRKCEKYKKYISEKKQEWDKQKTKYENKYVGKSASDLLKENYPECISANFDFIFNDNIEYKTYYPYGDYSSICSCEQVKYYKYNNAEKKNNKSLCYEKDNDMTWSKKYIKKLENGRSLEGVYVPPRRQQLCLYELFPIIIKNEEGMEKAKEELLETLQIVAEREAYYLWKQYNPTGKGIDDANKKACCAIRGSFYDLEDIIKGNDLVHDEYTKYIDSKLNEIFGSSDTNDIDTKRARTDWWENETITNGTDRKTIRQLVWDAMQSGVRYAVEEKNENFPLCMGVEHIGIAKPQFIRWLEEWTNEFCEKYTKYFEDMKSKCDPPKRADTCGDNSNIECKKACANYTNWLNPKRIEWNGMSNYYNKIYRKSNKESEGGKDYSMIMAPTVIDYLNKRCHGEINGNYICCSCKNIGAYNTTSGTVNKKLQKKETECEEEKGPLDLMNEVLNKMDKKYSAHKMKCTEVYLEHVEEQLNEIDNAIKDYKLYPLDRCFDDQTKMKVCDLIADAIGCKDKTKLDELDEWNDMDLRGTYNKHKGVLIPPRRRQLCFSRIVRGPANLRSLNEFKEEILKGAQSEGKFLGNYYKEHKDKEKALEAMKNSFYDYEDIIKGTDMLTNIEFKDIKIKLDRLLEKETNNTKKAEDWWKTNKKSIWNAMLCGYKKSGNKIIDPSWCTIPTTETPPQFLRWIKEWGTNVCIQKQEHKEYVKSKCSNVTNLGAQASESNNCTSEIKKYQEWSRKRSIRWETISKRYKKYKRMDILKDVKEPDANTYLREHCSKCPCGFNDMEEMNNNEDNEKEAFKQIKEQVKIPAELEDVIYRIKHHEYDKGNDYICNKYKNIHDRMKKNNGNFVTDNFVKKSWEISNGVLIPPRRKNLFLYIDPSKICEYKKDPKLFKDFIYWSAFTEVERLKKAYGGARAKVVHAMKYSFTDIGSIIKGDDMMEKNSSDKIGKILGDTDGQNEKRKKWWDMNKYHIWESMLCGYREAEGDTETNENCRFPDIESVPQFLRWFQEWSENFCDRRQKLYDKLNSECISAECTNGSVDNSKCTHACVNYKNYILTKKTEYEIQTNKYDNEFKNKNSNDKDAPDYLKEKCNDNKCECLNKHIDDKNKTWKNPYETLEDTFKSKCDCPKPLPSPIKPDDLPPQADEPFDPTILQTTIPFGIALALGSIAFLFMKVIYIYIYVCCICMYVCMYVCMYVCM

SEQ ID NO: 136 - protein, 640 aa, synthetic construct
  REGION 1..640: note = VAR2CSA truncate
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLD

SEQ ID NO: 137 - protein, 19 aa, synthetic construct
GGGGSGGGGSGGGSGGGGS

SEQ ID NO: 138 - protein, 18 aa, synthetic construct
GGGGSGGGGSGGGGSGGG

SEQ ID NO: 139 - protein, 8 aa, synthetic construct
GGGSGGGS